SEQUENCE LISTING (173 sequences)

SEQ ID NO: 1 - protein, 34 aa, Bacillus sp.
  MOD_RES (2)..(2): Alpha,beta-didehyrobutyric acid
IXAIXLAXPGAKXGALMGANMKXAXANASIHVXK

SEQ ID NO: 2 - protein, 21 aa, Bacillus sp.
  MOD_RES (8)..(8): 2-aminobutyric acid
IAAKFLAXPGAAKXGAFNAYA

SEQ ID NO: 3 - protein, 33 aa, Bacillus sp.
  MOD_RES (15)..(15): Alpha,beta-didehyrobutyric acid
AGPAIRAAVKQAQKXLKAXRLFXVAAKGKNGAK

SEQ ID NO: 4 - protein, 34 aa, Bacillus sp.
  MOD_RES (2)..(2): Alpha,beta-didehyrobutyric acid
IXAIXLAXPGAKXGALMGANMKXAXAHASIHVXK

SEQ ID NO: 5 - protein, 42 aa, Artificial Sequence
  Description of Artificial Sequence Synthetic peptide
CKHNGQHGGTAWAMAGNNIICTTAKGWDVSSHKGTVGNGYYK

SEQ ID NO: 6 - protein, 19 aa, Streptoverticillium cinnamoneus
CANSCSYGPLTWSCDGNTK

SEQ ID NO: 7 - protein, 34 aa, Bacillus sp.
  MOD_RES (2)..(2): Alpha,beta-didehyrobutyric acid
IXAIXLAXPGAKXGVLMGANMKXAXANASIHVXK

SEQ ID NO: 8 - protein, 32 aa, Bacillus sp.
  MOD_RES (5)..(5): Alpha,beta-didehydroalanine
WKAEXLAXPGAVXGALQXAFLQXLXANAKIXK

SEQ ID NO: 9 - protein, 32 aa, Bacillus sp.
  MOD_RES (5)..(5): Alpha,beta-didehydroalanine
WKAEXVAXPGAVXGVLQXAFLQXLXANAHIXK

SEQ ID NO: 10 - protein, 29 aa, Bacillus sp.
  MOD_RES (5)..(5): Alpha,beta-didehydroalanine
VLAKXLAXPGAIXGPLQXAYLAFPXFAKA

SEQ ID NO: 11 - protein, 21 aa, Bacillus sp.
  MOD_RES (8)..(8): 2-aminobutyric acid
IAAKFIAXPGAAKXGAFNAYA

SEQ ID NO: 12 - protein, 21 aa, Bacillus sp.
  MOD_RES (8)..(8): 2-aminobutyric acid
VAAKFLAXPGAAKXGAFNAYA

SEQ ID NO: 13 - protein, 21 aa, Bacillus sp.
  MOD_RES (5)..(5): Alpha,beta-didehydroalanine
FKAWXLAXPGAARXGAFNAYA

SEQ ID NO: 14 - protein, 21 aa, Bacillus sp.
  MOD_RES (5)..(5): Alpha,beta-didehydroalanine
FKAWXFAXPGAAKXGAFNAYA

SEQ ID NO: 15 - protein, 23 aa, Bacillus sp.
  MOD_RES (2)..(2): Alpha,beta-didehydroalanine
FXALXLAALGATGVKNPAFNAYA

SEQ ID NO: 16 - protein, 23 aa, Bacillus sp.
  MOD_RES (8)..(8): 2-aminobutyric acid
VGARYLAXPGXAWKLVAFXXXVK

SEQ ID NO: 17 - protein, 19 aa, Bacillus sp.
  MOD_RES (2)..(2): 2-aminobutyric acid
AXFXLPGGGGVAXLXXEAI

SEQ ID NO: 18 - protein, 19 aa, Bacillus sp.
  MOD_RES (7)..(7): 2-aminobutyric acid
ASGWVAXLXIEAGXVIAAA

SEQ ID NO: 19 - protein, 22 aa, Bacillus sp.
  MOD_RES (9)..(9): 2-aminobutyric acid
KRGSGWIAXIXDDAPNAVFVAA

SEQ ID NO: 20 - protein, 22 aa, Bacillus sp.
  MOD_RES (9)..(9): 2-aminobutyric acid
KKGSGWLAXIXDDAPNAVFVAA

SEQ ID NO: 21 - protein, 27 aa, Bacillus sp.
  MOD_RES (9)..(9): 2-aminobutyric acid
KGGSGVIHXIAHEANMNAWQFVFXAAS

SEQ ID NO: 22 - protein, 28 aa, Bacillus sp.
  MOD_RES (11)..(11): 2-aminobutyric acid
NRWWQGGVVPXVAYEARMNAWQHVFXAA

SEQ ID NO: 23 - protein, 26 aa, Bacillus sp.
  MOD_RES (8)..(8): 2-aminobutyric acid
GKNGVFKXIAHEAHLNXWAFLAXAAS

SEQ ID NO: 24 - protein, 25 aa, Bacillus sp.
  MOD_RES (7)..(7): 2-aminobutyric acid
GSGVIPXIAHEAHMNAFQFVFXAAS

SEQ ID NO: 25 - protein, 26 aa, Bacillus sp.
  MOD_RES (8)..(8): 2-aminobutyric acid
GGNGVIKXIAHEAHMNXWQFIFXAAS

SEQ ID NO: 26 - protein, 27 aa, Bacillus sp.
  MOD_RES (9)..(9): 2-aminobutyric acid
KKKSGVIPXVAHDAHMNAFQFVFXAAS

SEQ ID NO: 27 - protein, 27 aa, Bacillus sp.
  MOD_RES (13)..(13): 2-aminobutyric acid
KKTKKNASGDIAXLXXEADHLAXWVAA

SEQ ID NO: 28 - protein, 29 aa, Bacillus sp.
  MOD_RES (19)..(19): 2-aminobutyric acid
KAKWWNIAADLGNNGHVAXLAHGAQVSAN

SEQ ID NO: 29 - protein, 30 aa, Bacillus sp.
  MOD_RES (3)..(3): Alpha,beta-didehyrobutyric acid
AAXNXFXLADYWGNKGNWAXAXHEAMSWAK

SEQ ID NO: 30 - protein, 19 aa, Streptomyces cinnamoneus
  MOD_RES (11)..(11): 2-aminobutyric acid
ARQAAAFGPFXFVADGNXK

SEQ ID NO: 31 - protein, 19 aa, Streptomyces cinnamoneus
  MOD_RES (11)..(11): 2-aminobutyric acid
ALQAAAFGPFXFVADGNXK

SEQ ID NO: 32 - protein, 19 aa, Streptomyces cinnamoneus
  MOD_RES (11)..(11): 2-aminobutyric acid
ARQAAAFGPLXFVADGNXK

SEQ ID NO: 33 - protein, 19 aa, Streptomyces cinnamoneus
  MOD_RES (11)..(11): 2-aminobutyric acid
AANAAAYGPLXWSADGNXK

SEQ ID NO: 34 - protein, 19 aa, Streptomyces cinnamoneus
  MOD_RES (6)..(6): Alpha,beta-didehydroalanine
AVQAAXFGPLXWSADGNXK

SEQ ID NO: 35 - protein, 29 aa, Bacillus sp.
  MOD_RES (8)..(8): 2-aminobutyric acid
LGPAIKAXRQVAPKATRFVXVAAKKSDAQ

SEQ ID NO: 36 - protein, 30 aa, Bacillus sp.
  MOD_RES (2)..(2): Alpha,beta-didehydroalanine
AXVLKXXIKVAKKYAKGVXLXAGANIXGGK

SEQ ID NO: 37 - protein, 30 aa, Bacillus sp.
  MOD_RES (2)..(2): Alpha,beta-didehydroalanine
AXIVKXXIKAAKKLARGFXLXAGAHFXGKK

SEQ ID NO: 38 - protein, 31 aa, Bacillus sp.
  MOD_RES (5)..(5): Alpha,beta-didehyrobutyric acid
GIPCXIGAAVAAAIAVAPXTKAAKRAGKRKK

SEQ ID NO: 39 - protein, 33 aa, Bacillus sp.
  MOD_RES (2)..(2): Alpha,beta-didehyrobutyric acid
GXPLSXLLGGAAXGVIGYIANQTAPXTAAXRAA

SEQ ID NO: 40 - protein, 37 aa, Bacillus sp.
  MOD_RES (16)..(16): Alpha,beta-didehydroalanine
GLGKAQCAALWLQCAXGGXIGAGGGAVACQNYRQFCR

SEQ ID NO: 41 - protein, 21 aa, Bacillus sp.
  MOD_RES (6)..(6): Alpha,beta-didehydroalanine
TGARAXLLLAGDASLXITTAN

SEQ ID NO: 42 - protein, 21 aa, Bacillus sp.
  MOD_RES (1)..(1): Twofold methylated alanine
XXPAXPXVAQFVXQGSXXALV

SEQ ID NO: 43 - protein, 22 aa, Serratia marcescens
MSGGDGRGPGNSGLGHNGGQAR

SEQ ID NO: 44 - protein, 41 aa, Carnobacterium piscicola
MKIKTITKKQLIQIKGGSKNSQIGKSTSSISKCVFSFFKKC

SEQ ID NO: 45 - protein, 48 aa, Carnobacterium piscicola
MNKEFKSLNEVEMKKINGGSAILAITLGIFATGYGMGVQKAINDRRKK

SEQ ID NO: 46 - protein, 46 aa, Salmonella enteritidis
KRGRAPYSLIRQQVGGRWTYEIPHVGKIQYGGMVFDVDNLMINTPK

SEQ ID NO: 47 - protein, 46 aa, Salmonella enterica; identical to SEQ ID NO: 46

SEQ ID NO: 48 - protein, 46 aa, Salmonella typhimurium; identical to SEQ ID NO: 46

SEQ ID NO: 49 - protein, 46 aa, Salmonella paratyphi; identical to SEQ ID NO: 46

SEQ ID NO: 50 - protein, 46 aa, Salmonella typhi; identical to SEQ ID NO: 46

SEQ ID NO: 51 - protein, 48 aa, Lactobacillus plantarum
MKIQIKGMKQLSNKEMQKIVGGKSSAYSLQMGATAIKQVKKLFKKWGW

SEQ ID NO: 52 - protein, 37 aa, Lactococcus lactis
TSYGNGVHCNKSKCWIDVSELETYKAGTVSNPKDILW

SEQ ID NO: 53 - protein, 42 aa, Lactobacillus sakei
  MOD_RES (32)..(32): Any amino acid
TKYYGNGVYCNSKKCWVDWGQAAGGIGQTVVXGWLGGAIPGK

SEQ ID NO: 54 - protein, 43 aa, Leuconostoc mesenteroides
KNYGNGVHCTKKGCSVDWGYAWTNIANNSVMNGLTGGNAGWHN

SEQ ID NO: 55 - protein, 43 aa, Carnobacterium divergens
TKYYGNGVYCNSKKCWVDWGTAQGCIDVVIGQLGGGIPGKGKC

SEQ ID NO: 56 - protein, 43 aa, Enterococcus mundtii
KYYGNGVSCNKKGCSVDWGKAIGIIGNNSAANLATGGAAGWSK

SEQ ID NO: 57 - protein, 41 aa, Lactobacillus sakei
  MOD_RES (9)..(9): Any amino acid
KYYGNGVHXGKHSXTVDWGTAIGNIGNNAAANXATGXNAGG

SEQ ID NO: 58 - protein, 30 aa, Lactobacillus curvatus
AYPGNGVHCGKYSCTVDKQTAIGNIGNNAA

SEQ ID NO: 59 - protein, 31 aa, Lactobacillus paracasei
GMSGYIQGIPDFLKGYLHGISAANKHKKGRL

SEQ ID NO: 60 - protein, 35 aa, Lactococcus lactis
KKWGWLAWVDPAYEFIKGFGKGAIKEGNKDKWKNI

SEQ ID NO: 61 - protein, 39 aa, Lactococcus lactis
GTWDDIGQGIGRVAYWVGKAMGNMSDVNQASRINRKKKH

SEQ ID NO: 62 - protein, 46 aa, Lactobacillus acidophilus
MISSHQKTLTDKELALISGGKTHYPTNAWKSLWKGFWESLRYTDGF

SEQ ID NO: 63 - protein, 9 aa, Serratia plymuthica
DYHHGVRVL

SEQ ID NO: 64 - protein, 22 aa, Serratia plymuthica
ALPKKLKYLNLFNDGFNYMGVV

SEQ ID NO: 65 - protein, 38 aa, Lactobacillus curvatus
  MOD_RES (34)..(34): Any amino acid
YTAKQCLQAIGSCGIAGTGAGAAGGPAGAFVGAXVVXI

SEQ ID NO: 66 - protein, 31 aa, Leuconostoc mesenteroides
KGKGFWSWASKATSWLTGPQQPGSPLLKKHR

SEQ ID NO: 67 - protein, 44 aa, Pediococcus acidilactici
  MOD_RES (18)..(18): Any amino acid
KYYGNGVTCGKHSCSVDXGKATTCIINNGAMAXATGGHQGNHKC

SEQ ID NO: 68 - protein, 19 aa, Lactobacillus acidophilus
SRTPIIAGNWKLNMNPKET

SEQ ID NO: 69 - protein, 44 aa, Lactobacillus sakei
MMIFKKLSEKELQKISGGVGIQKCSLGFSSREYLNKITKWIKHH

SEQ ID NO: 70 - protein, 44 aa, Lactobacillus sakei; identical to SEQ ID NO: 69

SEQ ID NO: 71 - protein, 21 aa, Enterococcus faecium
  MOD_RES (12)..(12): Any amino acid
KYYGNGVTLNKXGXSVNXXXA

SEQ ID NO: 72 - protein, 36 aa, Leuconostoc mesenteroides
KYYGNGVHCTKSGCSVNWGEAASAGIHRLANGGNGF

SEQ ID NO: 73 - protein, 7 aa, Carnobacterium sp.
GSEIQPR

SEQ ID NO: 74 - protein, 46 aa, Streptococcus pyogenes
MNNTIKDFDLDLKTNKKDTATPYVGSRYLCTPGSCWKLVCFTTTVK

SEQ ID NO: 75 - protein, 42 aa, Rhizobium leguminosarum
MDNKVAKNVEVKKGSIKATFKAAVLKSKTKVDIGGSRQGCVA

SEQ ID NO: 76 - protein, 22 aa, Streptococcus mutans
FKSWSFCTPGCAKTGSFNSYCC

SEQ ID NO: 77 - protein, 19 aa, Streptomyces sp.
CVQSCSFGPLTWSCDGNTK

SEQ ID NO: 78 - protein, 19 aa, Streptomyces griseoluteus
CANSCSYGPLTWSCDGNTK

SEQ ID NO: 79 - protein, 19 aa, Streptomyces sp.
CRQSCSFGPLTFVCDGNTK

SEQ ID NO: 80 - protein, 19 aa, Streptomyces griseoverticillatus
CKQSCSFGPFTFVCDGNTK

SEQ ID NO: 81 - protein, 29 aa, Lactobacillus plantarum
  MOD_RES (9)..(9): Any amino acid
VYPFPGPIXMANLVLTXLSHLHRSTVXFS

SEQ ID NO: 82 - protein, 44 aa, Enterococcus faecium
ATRSYDNGIYCNNSKCWVNWGEAKENIAGIVISGWASGLAGMGH

SEQ ID NO: 83 - protein, 37 aa, Leuconostoc gelidum
KYYGNGVHCTKSGCSVNWGEAFSAGVHRLANGGNGFW

SEQ ID NO: 84 - protein, 44 aa, Lactobacillus plantarum
MVHQNVKFISRLLLASLLAAIVMGLSTAPIDILTLKYNWITVAI

SEQ ID NO: 85 - protein, 48 aa, Lactobacillus plantarum; identical to SEQ ID NO: 51

SEQ ID NO: 86 - protein, 48 aa, Lactobacillus plantarum
MLNKTINIIKKYPVRSLLVIVVFAIYVISDPSIISSFNQGLSDGAAGR

SEQ ID NO: 87 - protein, 44 aa, Pediococcus acidilactici
  MOD_RES (33)..(33): Any amino acid
KYYGNGVTCGKHSCSVDWGKATTCIINNGAMAXATGGHQGNHKX

SEQ ID NO: 88 - protein, 46 aa, Artificial Sequence
  Description of Artificial Sequence Synthetic construct
MDPTKYYGNGVYCNSKKCWVDWGQASGCIGQTVVGGWLGGAIPGKC

SEQ ID NO: 89 - protein, 50 aa, Enterococcus mundtii
MSQVVGGKYYGNGVSCNKKGCSVDWGKAIGIIGNNSAANLATGGAAGWKS

SEQ ID NO: 90 - protein, 30 aa, Lactococcus lactis
MKTILRFVAGYDIASHKKKTGGYPWERGKA

SEQ ID NO: 91 - protein, 47 aa, Lactobacillus plantarum
MDKFEKISTSNLEKISGGDLTTKLWSSWGYYLGKKARWNLKHPYVQF

SEQ ID NO: 92 - protein, 30 aa, Lactococcus lactis; identical to SEQ ID NO: 90

SEQ ID NO: 93 - protein, 46 aa, Lactobacillus acidophilus; identical to SEQ ID NO: 62

SEQ ID NO: 94 - protein, 43 aa, Enterococcus faecalis
MGAIAKLVAKFGWPFIKKFYKQIMQFIGQGWTIDQIEKWLKRH

SEQ ID NO: 95 - protein, 43 aa, Enterococcus faecium
MGAIAKLVTKFGWPLIKKFYKQIMQFIGQGWTIDQIEKWLKRH

SEQ ID NO: 96 - protein, 44 aa, Enterococcus faecium
MGAIAKLVAKFGWPIVKKYYKQIMQFIGEGWAINKIIEWIKKHI

SEQ ID NO: 97 - protein, 34 aa, Enterococcus faecium
MNFLKNGIAKWMTGAELQAYKKKYGCLPWEKISC

SEQ ID NO: 98 - protein, 34 aa, Lactobacillus curvatus; identical to SEQ ID NO: 97

SEQ ID NO: 99 - protein, 41 aa, Lactobacillus plantarum
ARSYGNGVYCNNKKCWVNRGEATQSIIGGMISGWASGLAGM

SEQ ID NO: 100 - protein, 47 aa, Leuconostoc mesenteroides; identical to SEQ ID NO: 91

SEQ ID NO: 101 - protein, 38 aa, Lactobacillus curvatus; identical to SEQ ID NO: 65
  MOD_RES (34)..(34): Any amino acid

SEQ ID NO: 102 - protein, 41 aa, Lactobacillus bavaricus; identical to SEQ ID NO: 57
  MOD_RES (9)..(9): Any amino acid

SEQ ID NO: 103 - protein, 24 aa, Lactobacillus acidophilus
  MOD_RES (9)..(9): Any amino acid
KTHYPTNAXKSLRKGFXESLRXTD

SEQ ID NO: 104 - protein, 22 aa, Lactococcus lactis
FITSSKNKNLGGGLIMSWGRLF

SEQ ID NO: 105 - protein, 35 aa, Lactococcus lactis; identical to SEQ ID NO: 60

SEQ ID NO: 106 - protein, 39 aa, Lactococcus lactis; identical to SEQ ID NO: 61

SEQ ID NO: 107 - protein, 44 aa, Artificial Sequence
  Description of Artificial Sequence Synthetic construct
KYYGNGVTCGKHSCSVDWGKATTCIINNGAMAWATGGHQGNHKC

SEQ ID NO: 108 - protein, 49 aa, Escherichia coli
MKKAKAIFLFILIVSGFLLVACQANYIRDVQGGTVAPSSSSELTGIAVQ

SEQ ID NO: 109 - protein, 37 aa, Unknown; identical to SEQ ID NO: 83
  Description of Unknown Synthetic peptide

SEQ ID NO: 110 - protein, 24 aa, Lactobacillus plantarum
KKKKQSWYAAAGDAIVSFGEGFLN

SEQ ID NO: 111 - protein, 26 aa, Lactobacillus plantarum
  MOD_RES (1)..(1): Any amino acid
XNKLAYNMGWYAGXATIFGLAAXALL

SEQ ID NO: 112 - protein, 17 aa, Enterococcus faecium
MKNNKSFNKILTETATP

SEQ ID NO: 113 - protein, 45 aa, Carnobacterium piscicola
MNDKKYLKLKECSEKKLKQIQGGNKSVIKGNPNLAQCVFSFFKKC

SEQ ID NO: 114 - protein, 48 aa, Carnobacterium maltaromaticum
  MOD_RES (1)..(1): Any amino acid
XNYGNGVSCSKTKCSVNWGQAFQERYTAGINSFVSGVASGAGSIGRRP

SEQ ID NO: 115 - protein, 37 aa, Leuconostoc gelidum; identical to SEQ ID NO: 83

SEQ ID NO: 116 - protein, 26 aa, Enterococcus hirae
MAFYLPYLLIFVSISGSIWLIYKIFQ

SEQ ID NO: 117 - protein, 8 aa, Unknown
  Description of Unknown Synthetic peptide
LSGGQXQR

SEQ ID NO: 118 - protein, 8 aa, Unknown
  Description of Unknown Synthetic peptide
GXXGXGKX

SEQ ID NO: 119 - protein, 38 aa, Unknown
  Description of Unknown Synthetic peptide
VPGGCTYTRSNRDVIGTCKTGSGQFRIRLDCNNAPDKT

SEQ ID NO: 120 - protein, 44 aa, Enterococcus faecium
ATRSYGNGVYCNNSKCWVNWGEAKENIAGIVISGWASGLAGMGH

SEQ ID NO: 121 - protein, 44 aa, Enterococcus faecium
MCSRSSQEYVSRYQLLILKVDRIPFPIAFILPKKGEQLNRRTFI

SEQ ID NO: 122 - protein, 46 aa, Streptococcus gordonii
MKEFKELSKQELEKTCGGVAMPALWFFRRQAPSGNRRSSRFSLLIL

SEQ ID NO: 123 - protein, 40 aa, Enterococcus faecium
MRENGQKPKRSAKKTYQAPQAKKVRVTSRKEKFLEQLLKI

SEQ ID NO: 124 - protein, 33 aa, Enterococcus faecium
MYLSTYYPCTPHDKWAEGLAALGIKGIIRLPGF

SEQ ID NO: 125 - protein, 41 aa, Staphylococcus aureus
MKQLDIPQLLIINGGSGGNYTLPGQPKGDIKKCILSFFKNC

SEQ ID NO: 126 - protein, 32 aa, Lactobacillus casei
MLKSIFTLLIAPVLAGIAISLFDHWLDDQGRK

SEQ ID NO: 127 - protein, 44 aa, Enterococcus faecalis
MLAKIKAMIKKFPNPYTLAAKLTTYEINWYKQQYGRYPWERPVA

SEQ ID NO: 128 - protein, 47 aa, Lactobacillus plantarum
MDKIIKFQGISDDQLNAVIGGKKKKQSWYAAAGDAIVSFGEGFLNAW

SEQ ID NO: 129 - protein, 45 aa, Lactobacillus sakei
MKLNYIEKKQLTNKQLKLIIGGTNRNYGKPNKDIGTCIWSGFRHC

SEQ ID NO: 130 - protein, 43 aa, Streptococcus dysgalactiae
MKKRLTITLSDSVLENLEKMAKEMGLSKSAMISVENYKKGQEK

SEQ ID NO: 131 - protein, 22 aa, Unknown
  Description of Unknown Synthetic peptide
FKSWSFCTPGCAKTGSFNSYCC

SEQ ID NO: 132 - protein, 46 aa, Streptococcus mutans
MKKTLSLKNDFKEIKTDELEIIIGGSGSLSTFFRLFNRSFTQALGK

SEQ ID NO: 133 - protein, 46 aa, Streptococcus mutans
MKKTPSLKNDFKEIKTDELEIIIGGSGSLSTFFRLFNRSFTQALGK

SEQ ID NO: 134 - protein, 46 aa, Streptococcus mutans
MKGTLSLKNDFKEIKTDELEIIIGGSGSLSTFFRLFNRSFTQALGK

SEQ ID NO: 135 - protein, 48 aa, Streptococcus mutans
MKKTPSLKNDFKEIKTDELEIIIGGSGSLSTFFRLFNRSFTQALGKIR

SEQ ID NO: 136 - protein, 43 aa, Streptococcus mutans
MKKTLSLKNDFKEIKTDELEIITGGSGSLSTFFRLFNRSFTQA

SEQ ID NO: 137 - protein, 43 aa, Streptococcus mutans
MKKTLSLKNDFKEIKTDELEIIIGGSGSLSTFFRLFNRSFTQA

SEQ ID NO: 138 - protein, 49 aa, Lactobacillus plantarum
MKNINKYTELNDQKLQSLIGGKTKTISLMSGLQVPHAFTKLLKALGGHH

SEQ ID NO: 139 - protein, 50 aa, Lactobacillus plantarum
MLNKTINIIKKYPVRSLLVVLIVVFAIYVISDPSIISSFNQGLSDGTAGR

SEQ ID NO: 140 - protein, 48 aa, Lactobacillus plantarum
MKIQIKSMKQLSNKEMQKIVGGKSSAYSLQMGATAIKQVKKLFKKWGW

SEQ ID NO: 141 - protein, 37 aa, Lactobacillus plantarum
MLNKTINIIKKYPVRSLLVVLIVVFAIYVISDGAAGR

SEQ ID NO: 142 - protein, 41 aa, Pediococcus acidilactici
  MOD_RES (13)..(14): Any amino acid
MGAIAKLVAKFGXXIVVKYYKQIMQFIGQGVTINXIPLIXF

SEQ ID NO: 143 - protein, 24 aa, Lactobacillus acidophilus
GNPKVAHCASQIGRSTAWGAVSGA

SEQ ID NO: 144 - protein, 23 aa, Lactobacillus acidophilus
NPKVAHCASQIGRSTAWGAVSGA

SEQ ID NO: 145 - protein, 50 aa, Bacillus subtilis
MKLPVQQVYSVYGGKDLPKGHSHSTMPFLSKLQFLTKIYLLDIHTQPFFI

SEQ ID NO: 146 - protein, 44 aa, Streptococcus ratti
MWGRILAFVAKYGTKAVQWAWKNKWFLLSLGEAVFDYIRSIWGG

SEQ ID NO: 147 - protein, 42 aa, Lactococcus lactis
ELAEVNGGSLQYVMSAGPYTWYKDTRTGKTICKQTIDTASYT

SEQ ID NO: 148 - protein, 22 aa, Lactobacillus helveticus
MHNSIAYDKDGNSTGQKYYAYG

SEQ ID NO: 149 - protein, 27 aa, Artificial Sequence
  Description of Artificial Sequence Synthetic peptide
MLVFQMRNVDKTSTVLKQTKNSDYADK

SEQ ID NO: 150 - protein, 37 aa, Unknown
  Description of Unknown Synthetic peptide
KYYGNGVSCNSHGCSVNWGQAWTCGVNHLANGGHGVC

SEQ ID NO: 151 - protein, 49 aa, Clostridium beijerinckii
MNKKKLLIYAILFLIYIILFLTYNNSIFRIILVVSLGFLSSIISKLQIK

SEQ ID NO: 152 - protein, 25 aa, Lactobacillus acidophilus
  MOD_RES (14)..(14): Any amino acid
RNNWQTNVGGAVGXAMIGATVGGTI

SEQ ID NO: 153 - protein, 45 aa, Lactobacillus sakei
MTNRKTLPKEELKKIKGGTPGGFDIISGGPHVAQDVLNAIKDFFK

SEQ ID NO: 154 - protein, 49 aa, Klebsiella pneumoniae
MKKVKTIFLFILIASGFLLVACQANYNRDVQGGTVAPSSSSELTGIAVQ

SEQ ID NO: 155 - protein, 38 aa, Lactobacillus salivarius
MKFEVLTEKKLQKIAGGATKKGGFKRWQCIFTFFGVCK

SEQ ID NO: 156 - protein, 50 aa, Lactobacillus salivarius
MLKKLWNIWLDGGLIRGRKRYVIIPIIFAIFLPLSMWLSDNEGMSYLDYI

SEQ ID NO: 157 - protein, 29 aa, Staphylococcus aureus
MGAVIKVGAKVIGWGAASGAGGLEKILKK

SEQ ID NO: 158 - protein, 31 aa, Staphylococcus aureus
MGALIKTGAKIIGSGAAGGLGTYIGHKILGK

SEQ ID NO: 159 - protein, 30 aa, Staphylococcus aureus
MGAVAKFLGKAALGGAAGGATYAGLKKIFG

SEQ ID NO: 160 - protein, 31 aa, Staphylococcus aureus
MGKLAIKAGKIIGGGIASALGWAAGEKAVGK

SEQ ID NO: 161 - protein, 30 aa, Clostridium botulinum
MEFKNKQRMYREFFMTLKESFKFSSKKRYI

SEQ ID NO: 162 - protein, 31 aa, Lactobacillus gasseri
MLDKNIDLQRAIFHIKQDINKVVYGFKLPET

SEQ ID NO: 163 - protein, 26 aa, Lactococcus lactis
MKKKFQDSISNSVYKYRVLSRLSQQD

SEQ ID NO: 164 - protein, 23 aa, Lactobacillus helveticus
MHNSIAYDKDGNSTGQKYYAYGS

SEQ ID NO: 165 - protein, 40 aa, Lactococcus lactis
MLIKVLEKKSYLRMLQLTLIEIVYISLWHPMVQGKQPFLR

SEQ ID NO: 166 - protein, 35 aa, Lactobacillus sakei
MMIFKKLSEKELQKINGGMAGNSSNFIHKIKQIFT

SEQ ID NO: 167 - protein, 42 aa, Carnobacterium piscicola
MKNFFKKNNMLYRFFAVIGLIFGGWALFNIAMFIGRSIGSLF

SEQ ID NO: 168 - protein, 21 aa, Enterococcus faecalis
MKQQKKHIAALLFALILTLVS

SEQ ID NO: 169 - protein, 36 aa, Actinobacillus pleuropneumoniae
MEPNKNKDLGLAALKILAQYHNISVNPEELKHKFDL

SEQ ID NO: 170 - protein, 4 aa, Enterococcus faecalis
KRLE

SEQ ID NO: 171 - protein, 45 aa, Enterococcus faecalis
QDDISSIKCIYKNRLLKVGLIFVLASAGGAIGNIIGGIELFKNLI

SEQ ID NO: 172 - protein, 19 aa, Enterococcus faecalis
KKSNSNTPGVITHNWVENQ

SEQ ID NO: 173 - protein, 6 aa, Artificial Sequence
  Description of Artificial Sequence Synthetic 6xHis tag
HHHHHH

(19) United States
(12) Patent Application Publication
(10) Pub. No.: US 2011/0150917 A1
(43) Pub. Date: Jun. 23, 2011

(54) Small Cationic Antimicrobial Peptides

(75) Inventors: Robert E. Hancock, Vancouver (CA); Hans-Georg Sahl, Bonn (DE); Melissa Elliott, Vancouver (CA)

(73) Assignee: THE UNIVERSITY OF BRITISH COLUMBIA, Vancouver (CA)

(21) Appl. No.: 12/664,282

(22) PCT Filed: Jun. 12, 2008
(86) PCT No.: PCT/CA2008/001129

§ 371 Date: Mar. 7, 2011

Related U.S. Application Data
(60) Provisional application No. 60/929,086, filed on Jun. 12, 2007.

(51) Int. Cl. A61K 39/02; C07K 2/00; C07K 14/195; C07K 7/08; C12N 15/31; C12N 5/00; C12Q 1/68; A61K 31/7052; A61P 37/04; A61P 31/04; A61P 29/00

(52) U.S. Cl. 424/190.1; 530/300; 530/324; 530/326; 536/23.7; 435/375; 435/6.13; 514/44 R

(57) ABSTRACT

Cationic bacteriocin and lantibiotic peptides are provided and their immunomodulatory activities are described. Methods are provided for selectively enhancing innate immunity. Other methods are provided for selectively suppressing a proinflammatory response. Other methods are provided for identifying a compound or compounds which modulate an innate immune response. Pharmaceutical compositions comprising the cationic bacteriocin and lantibiotic peptides or polynucleotides are also provided.

### CROSS-REFERENCE TO RELATED APPLICATIONS

This application claims the benefit of priority to U.S. provisional patent application Ser. No. 60/929,086, filed Jun. 12, 2007, the disclosure of which is incorporated by reference in its entirety.

### FIELD

The present invention relates generally to peptides and more specifically to immunomodulatory lantibiotic and bacteriocin peptides.

### BACKGROUND

The treatment of bacterial infections with antibiotics is one of the mainstays of human medicine. Unfortunately the effectiveness of antibiotics has become limited due to an increase in bacterial antibiotic resistance in the face of a decreasing efforts and success in discovery of new classes of antibiotics. Today, infectious diseases are the second leading cause of death worldwide and the largest cause of premature deaths and loss of work productivity in industrialized countries. Nosocomial bacterial infections that are resistant to therapy result in annual costs of more than $2 billion and account for more than 80,000 direct and indirect deaths in North America alone, whereas a major complication of microbial diseases, namely sepsis, accounts for 700,000 cases and 140,000 deaths in North America.

Immunity is generally considered to have two major arms, innate immunity and adaptive immunity. Adaptive immunity includes the humoral (antibody-based) and cellular (activated T-cell based) immune responses and features, as hallmarks, exquisite antigen specificity driven by gene rearrangements, memory such that each succeeding response to a given antigen reflects the history of prior responses, and self vs. non-self discrimination. It takes time for adaptive immunity to be triggered, at least 3-7 days, but the clonal expansion of key antigen-specific lymphocytes makes this response highly effective in dealing with specific pathogens. In contrast, innate immunity is either immediately available or rapidly activated, works through non-rearranging receptors (e.g., Toll-like receptors; TLR), that recognize conserved microbial signature molecules, and is relatively non-specific. The two systems are interconnected in two ways, (A) the effector mechanisms for destroying pathogens are largely shared, and (B) “innate immunity instructs adaptive immunity”, in that there are mechanisms for ensuring a transition to adaptive immunity, if innate immunity fails to control infections. Innate immunity can be boosted to become more effective but this can lead to a double-edged sword with a co-boosting of potentially harmful inflammation, and in extreme cases, sepsis.

The innate immune system is a highly effective and evolved general defense system that involves a variety of effector functions including phagocytic cells, complement, and the like, but is generally incompletely understood. Elements of innate immunity are always present at low levels and are activated very rapidly when stimulated by pathogens, acting to prevent these pathogens from causing disease. Generally speaking many known innate immune responses are “triggered” by the binding of microbial signaling molecules, like lipopolysaccharide (LPS), with pattern recognition receptors such as Toll-like receptors (TLR) on the surface of host cells. Many of the effector functions of innate immunity are grouped together in the inflammatory response. However, too severe an inflammatory response can result in responses that are harmful to the body, and, in an extreme case, sepsis and potentially death can occur; indeed sepsis occurs in approximately 780,000 patients in North America annually with 140,000 deaths. Thus, a therapeutic intervention to boost innate immunity, which is based on stimulation of TLR signaling (for example using a TLR agonist), has the potential disadvantage that it could stimulate a potentially harmful inflammatory response and/or exacerbate the natural inflammatory response to infection.

One novel approach to antibacterial therapy is through the selective modulation of innate immunity using cationic host defence (also termed “antimicrobial”) peptides. Such peptides, found in most species of life, represent a template for a new therapy against infections. They selectively activate host innate immunity without displaying immunogenicity (Hancock R E W. 2001, Lancet Infectious Diseases 1: 156-164) while counteracting some of the more harmful aspects of inflammation (e.g. sepsis, endotoxaemia), which is extremely important since rapid killing of bacteria and subsequent liberation of bacterial components such as LPS or peptidoglycan can induce fatal immune dysregulation (Jarisch-Herxheimer reaction) (Gough M, Hancock R E W, Kelly N M. 1996, Infection and Immunity 64:4922-4927). Collectively host defence peptides have a broad range of immunomodulatory properties, including a variety of important effector functions such as the modulation of expression of hundreds of genes in monocytes, epithelial cells and the like, selective activation of innate immunity, promotion of angiogenesis and wound healing responses, chemoattraction of immune cells, induction of chemokines and differentiation responses, resolution of infections, and, with some peptides, an ability to rapidly and directly kill both bacteria and other microbes. Thus they offer multiple opportunities to treating infections with uses as broad spectrum antibiotics and/or as agents that selectively enhance aspects of innate immunity while suppressing potentially harmful inflammation.

Recently it was demonstrated (Scott, M. G., et al. 2007, Nature Biotechnology 25: 465-472) that a novel synthetic peptide based on natural host defence peptides from cattle, namely innate defence regulator peptide (IDR-1) was protective in murine models of infection with important Gram positive and Gram negative pathogens, including methicillin-resistant Staphylococcus aureus, vancomycin resistant enterococci, and Salmonella enterica. It was effective by both local and systemic administration, when given 48 h before or 6 h after infection. Unlike some antimicrobial host defence peptides, it was not directly antimicrobial and thus is unlikely to select for antimicrobial resistance. Gene and protein expression analysis in human monocytes and murine macrophages indicated that IDR-1, acting through mitogen activated protein kinase and other signalling pathways, enhanced the levels of monocyte chemokines while reducing pro-inflammatory cytokine responses. These mechanisms were demonstrated in a murine model of infection as evidenced by an increase in monocytes/macrophages and a reduction of inflammatory cytokines at the site of infection. Thus IDR-1 was the first member of a class of innate defence regulators which counter infection by selective modulation of innate immunity.

However one of the major issues with such peptides is the high cost of goods as natural peptides tend to be quite expensive ($100 to $200 per gram), making them too expensive to utilize therapeutically for infections in many of the poorer nations. Thus we considered here natural sources of peptides, in particular bacteriocin like the lantibiotics.

Bacterial peptides (termed bacteriocins), even when they contain one or two disulphide bonds, tend to be highly flexible in solution and adopt amphipathic structures only upon contact with membranes and membrane-mimicking environments. Among the bacteriocins of Gram-positive bacteria there is a particular group, the lantibiotics (lanthionine-containing peptide antibiotics), that are characterized by thioether-based intramolecular rings resulting from posttranslational modifications of serine (or threonine) and cysteine residues. Lanthionine-rings create segments of defined spatial structures in the peptides some of which represent conserved binding motifs for recognition of specific targets. These ring structures also provide stability against proteases, possibly including the antigen processing machinery since antibodies against highly cross-bridged lantibiotics such as gallidermin are very difficult to obtain.

Bacteriocins overcome some of the major issues with cationic host defence peptides including cost of goods since they are naturally produced recombinantly by bacteria and large scale fermentation and purification schemes have been developed. Also the lantibiotics which have unusual structures and amino acids are relatively resistant to proteases. In addition it can be assumed that they are relatively safe, at least when taken orally, since bacteriocins of lactic acid bacteria, in particular nisin, have a long and impressive history in food preservation. For such purposes, cost-effective semi-purified preparations such as Nisaplin™ are available; otherwise producing strains can be included directly in the food production process. Various clinical applications have also been considered (Cotter, P. D., et al. 2005. Nature Reviews Microbiology 3:777-88) including topical treatment of skin infections such as juvenile acne (gallidermin), bovine mastitis (nisin, lacticin 3147) and eradication of MRSA nasal colonization. However the only activity identified in such peptides to date of relevance to treatment of infections is direct antimicrobial activity.

### SUMMARY

The present invention is based on the discovery that certain cationic bacteriocin peptides are able to induce chemokine production in human peripheral blood mononuclear cells (PBMC), an activity that reflects the ability of peptides to protect against infection through selective modulation of innate immunity. Exemplary peptides of the invention include nisin Z, Pep5, gallidermin, Pediocin PA1, nisin A and duramycin.

The invention further provides isolated immunomodulatory bacteriocin or lantibiotic peptides with net cationic charge. In some aspects, the peptide has an amino acid sequence of SEQ ID NO: 1-6, or analogs, derivatives, amidated variations and conservative variations thereof.

The invention further provides methods of modulating the innate immune response of a cell or cells in a manner that enhances the production of a protective immune response while not inducing or inhibiting the potentially harmful proinflammatory response responsible for sepsis and harmful inflammation.

The invention further provides methods of selectively enhancing innate immunity comprising contacting a cell containing a gene that encodes a polypeptide involved in innate immunity and protection against an infection with an isolated immunomodulatory bacteriocin or lantibiotic peptide with net cationic charge, wherein expression of the gene in the presence of the bacteriocin or lantibiotic peptide is modulated as compared with expression of the gene in the absence of the bacteriocin or lantibiotic peptide, and wherein the modulated expression results in enhancement of innate immunity. In some aspects, the bacteriocin or lantibiotic peptide protects against an infectious agent. In other aspects the infectious agent is a bacterium. In some such aspects, the bacterium is selected from a group containing Staphylococcus aureus and Citrobacter rodentium. In some aspects, the innate immune response contributes to adjuvanticity leading to the promotion of a subsequent antibody response. In some aspects, the bacteriocin or lantibiotic peptide does not stimulate a septic reaction. In some such aspects, the bacteriocin or lantibiotic peptide stimulates expression of the one or more genes or proteins, thereby selectively enhancing innate immunity. In some aspects, the one or more genes or proteins encode chemokines or interleukins that attract immune cells. In some such aspects, the one or more genes are selected from the group consisting of MCP-1, MCP-3, IL-8, Gro-α or IL-6. In some aspects, the peptide is a member of the cationic bacteriocin family. In other aspects, the bacteriocin is from the subfamily of cationic lantibiotics. In some such aspects, the peptide is selected from the group consisting of SEQ ID NO: 1-6. In some aspects, the enhancement of innate immunity leads to a stimulation of adaptive immune responses to immunization with an antigen.

The invention further provides a method of selectively suppressing a proinflammatory response comprising contacting a cell containing a gene that encodes a polypeptide involved in inflammation and sepsis with an isolated immunomodulatory bacteriocin or lantibiotic peptide with net cationic charge, wherein the expression of the gene is modulated in the presence of the bacteriocin or lantibiotic peptide compared with expression in the absence of the bacteriocin or lantibiotic peptide, and wherein the modulated expression results in enhancement of innate immunity. In some aspects, the bacteriocin or lantibiotic peptide inhibits the inflammatory or septic response. In other aspects, the bacteriocin or lantibiotic peptide blocks the inflammatory or septic response. In other aspects, the bacteriocin or lantibiotic peptide inhibits the expression of a pro-inflammatory gene or molecule. In some such aspects, the bacteriocin or lantibiotic peptide inhibits the expression of TNF-α. In some aspects, the peptide is a member of the cationic bacteriocin family. In other aspects, the bacteriocin is from the subfamily of cationic lantibiotics. In some such aspects, the peptide is selected from the group consisting of SEQ ID NO: 1-6. In some aspects, the inflammation is induced by a microbe or a microbial ligand acting on a Toll-like receptor. In some such aspects, the microbial ligand is a bacterial endotoxin or lipopolysaccharide. In some the peptide is a member of the cationic bacteriocin family. In some such aspects, the bacteriocin is from the subfamily of cationic lantibiotics. In other aspects, the peptide is selected from the group consisting of SEQ ID NO: 1-6. In some aspects, the enhancement of innate immunity is further assisted by the co-administration of a conventional adjuvant. In some such aspects, the conventional adjuvant is an oligonucleotide containing the sequence motif CpG. In some aspects, the peptide is a member of the cationic bacteriocin family. In some such aspects, the bacteriocin is from the subfamily of cationic lantibiotics. In some such aspects, the peptide is selected from the group consisting of SEQ ID NO: 1-6.

The invention further provides a method for identifying a compound which modulates an innate immune response, the method comprising: (a) providing a cell-based assay system comprising a cell containing a gene that encodes a polypeptide involved in innate immunity and protection against infection, expression of the gene being modulated during an innate immune response; (b) contacting the cell with a test compound; and (c) measuring expression of the gene in the assay system, wherein a difference in expression in the presence of the compound relative to expression in the absence of the compound is indicative of modulation. In some aspects, the compound is an agonist of an innate immune response. In other aspects, the compound is an antagonist of an innate immune response. In some aspects, the compound is an inhibitor of an innate immune response. In other aspects, the compound is an activator of an innate immune response. In some aspects, the test compound is an organic molecule, a natural product, a peptide, an oligosaccharide, a nucleic acid, a lipid, an antibody, or binding fragment thereof. In other aspects, the test compound is from a library of compounds. In some aspects, the library is a random peptide library, a combinatorial library, an oligosaccharide library or a phage display library.

The invention further provides pharmaceutical compositions comprising the peptides or polynucleotides of the invention together with a pharmaceutically acceptable carrier.

### BRIEF DESCRIPTION OF THE DRAWINGS

FIG. 1. Sequences of the lantibiotic bacteriocins used. A. Sequences of the lantibiotic bacteriocins used. Nisin Z (SEQ ID NO: 1); Gallidermin (SEQ ID NO: 2); Pep5 (SEQ ID NO: 3); Nisin A (SEQ ID NO: 4), Pediocin PA1 (SEQ ID NO: 5), Duramycin (SEQ ID NO: 6). B. Peptides in bold type are prototype peptides; natural variants, subsequently described, are given in regular type. (A) Lantibiotics of the nisin group. (B) Lantibiotics of the mersacidin group. (C) Lantibiotics of the cinnamycin group. (D) Miscellaneous lantibiotics with only few variants and unknown molecular target. Unusual amino acids are: Ala-S-Ala, lanthionine; Abu-S-Ala, 3-methyllanthionine; Abu, 2-aminobutyric acid; Dha, α,β-didehydroalanine; Dhb, α,β-didehydrobutyric acid; Me₂A, twofold methylated alanine; aI, allo isoleucine; A*, alanine in the D-configuration. N-terminal modifications given in FIG. 3D occur spontaneously from Dha and Dhb after proteolytic cleavage. Legend: (_) residues conserved with respect to the prototype peptide; (−) missing residue.

FIG. 2A-C. Dose response of induction, by lantibiotic peptides, of chemokines in human PBMC.

FIG. 3A-C. Reinforcement of chemokine responses in human PBMC to the bacterial signature molecules, Cpg oligonucleotides, by co-administration of lantibiotic peptides. On the X-axis of this and subsequent Figures, N=Nisin Z, P=Pep5, G=gallidermin.

FIG. 4. Anti-endotoxic activity of cationic lantibiotic peptides and lack of ability of these peptides by themselves to induce expression of the pro-inflammatory cytokine TNFα.

FIG. 5. Lack of synergy between the lantibiotic peptides and high dose LPS to induce IL-6 and IL-8.

FIG. 6. Lack of cytotoxicity in PBMC for lantibiotics.

FIG. 7. Induction of Chemokines in Response to Nisin A and other peptides. Candidate Lantibiotic Immunomodulatory Activities. Various lantibiotics were screened for chemokine induction in human PBMCs. Cells were stimulated for 24 hours with 100 μg of peptide and supernatants were analyzed for chemokines by ELISA.

FIG. 8. Adjuvant Formulations with Nisin Z cf. control peptide 1002. Assessed as synergistic effect in MCP-1 release over and above the sum of the individual components where a number greater than 1 indicates synergy.

FIG. 9. Protection of animals vs. Staphylococcus aureus challenge when administered 4 hours prior to initiating the infection. A represents the reduction in colony counts within the peritoneum of mice treated with lantibiotic peptide (or negative control 1005 or positive control 1002) and challenged with ˜10⁸ S. aureus in hog gastric mucin. B represents the visual observation scores that were vastly improved by treatment with nisin. An additional peptide 1002 was included as a positive control.

FIG. 10. Live-time Non-invasive imaging following Citrobacter rodentium challenge; effect of nisin treatment. Animals were pre-treated intraperitoneally with 200 μg nisin, 4 h prior to initiating the infection. Mice were then infected via gavage with 2.5×10⁸ CFU of Citrobacter rodentium (lux—luminescence labeled). Live mice were followed with a CCD camera over time to assess bacterial clearance/resolution of infection. The imaging shows bacteria as a grayscale gradient (white to black—ringed in white in the first control mouse—where the black represents areas of infection and white represents very intent infection) and shows that superior clearance of S. aureus lasts for up to 11 days after injection of nisin.

FIG. 11. Histology of the intestines of Citrobacter treated animals after sacrifice at day 11; effect of nisin treatment. A. Sections of (from right to left): normal uninfected, saline treated infected, and nisin-treated intestines. Letter labels are a. Inflammatory infiltrate and edema; b. elongated crypts (hyperplasia); c. sloughing of damaged epithelial cells (mucosal integrity); d. depletion of mucous in goblet cells. B. Scoring of these micrographs. The significant (p<0.05) increase in inflammatory exudate and edema (reflecting increased recruitment of infection fighting immune cells) and decrease in damage to epithelial integrity and goblet cell depletion (reflecting an improvement in the functioning of the intestines) are positive outcomes of nisin treatment.

### DETAILED DESCRIPTION

### A. Introduction

Lantibiotics are well known for their direct antimicrobial activities but they have a rather narrow range of antibiotic activities. Thus while they have been used in food applications, their narrow range of activities (excellent activity against lactobacilli but moderate activity against many Gram positive pathogens and no activity against any Gram negative bacteria) has blocked their development as commercial antibiotics for human medicine. In contrast it is known that short cationic peptides have the capability for protecting against a broad range of bacterial infections by selectively stimulating innate immunity without enhancing pro-inflammatory responses (Scott, M. G., et al. 2007, Nature Biotechnology 25: 465-472.). Thus by screening for the appropriate immunomodulatory activities that underlie protection, we reasoned we should be able to find relatively inexpensive, protease-resistant and non-toxic peptides. Thus we initiated a screen of cationic bacteriocin and lantibiotic peptides. Table 1 provides candidate immunomodulatory peptides from which we chose candidate peptides.

The bacteriocin and lantibiotic peptides have also been examined for ability to induce chemokines in human peripheral blood mononuclear cells (equivalent to protective immunomodulatory activity) and demonstrate that this procedure can be used to screen cationic bacteriocin and lantibiotic peptides for these properties. This then indicates that the peptides have potential for modulating immunity.

The invention provides a number of methods, reagents, and compounds that can be used for screening for effective immunomodulators with anti-infective activity. It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Selective enhancement of innate immunity” as used herein means that the peptides of the invention are able to upregulate, in mammalian cells, genes and molecules that are natural components of the innate immune response and assist in the resolution of infections without excessive increases of pro-inflammatory cytokines like TNFα which can cause potentially harmful inflammation and thus stimulate a sepsis reaction in a subject. The peptides do not stimulate a septic reaction, but do stimulate expression of the one or more genes encoding chemokines or interleukins that attract immune cells including MCP-1, MCP-3, IL-8, and CXCL-1. The peptide can also possess anti-sepsis activity including an ability to reduce the expression of TNFα in response to bacterial ligands like LPS.

“Subject” or “patient” refers to any mammalian patient or subject to which the compositions of the invention can be administered. The term mammals, human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the invention.

The “amino acid” residues identified herein are in the natural L-configuration, except for the characteristic lanthionine and 3-methyllanthionine which are in the D,L-conformation and individual alanine residues e.g. in lacticin 3147 and lactocin S which occur in the D-configuration. In keeping with standard polypeptide nomenclature, Journal of Biological Chemistry 243:3557-59, (1969), abbreviations for amino acid residues are as shown in the following table (Table 1).

TABLE 1

| 1-Letter | 3-Letter | Amino Acid |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | He | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gin | L-glutamine |
| E | Glu | L-glutamic acid |
| W | Trp | L-tryptohan |
| R | Arg | L-arginine |
| D | Asp | L-aspartic acid |
| N | Asn | L-asparagine |
| C | Cys | L-cysteine |
| Lan | Lan | (2S,6R)-lanthionine |
| MeLan | MeLan | (2S,3S,6R)-3-methyllanthionine |
| Dha | Dha | 2,3-didehydroalanine |
| Dhb | Dhb | (Z)-2,3-didehydrobutyrine |

In addition to these amino acids a variety of unusual post translational modifications typical of lantibiotic peptides are included (lysinoalanine, β-hydroxy-aspartate, D-alanine, 2-oxobutyrate, 2-oxopropionate (pyruvate), 2-hydroxypropionate (lactate), S-aminovinyl-D-cysteine, and S-aminovinyl D-methylcysteine). It should be noted that all amino acid residue sequences are represented herein by formulae whose left to right orientation is in the conventional direction of amino-terminus to carboxy-terminus.

### B. Peptides

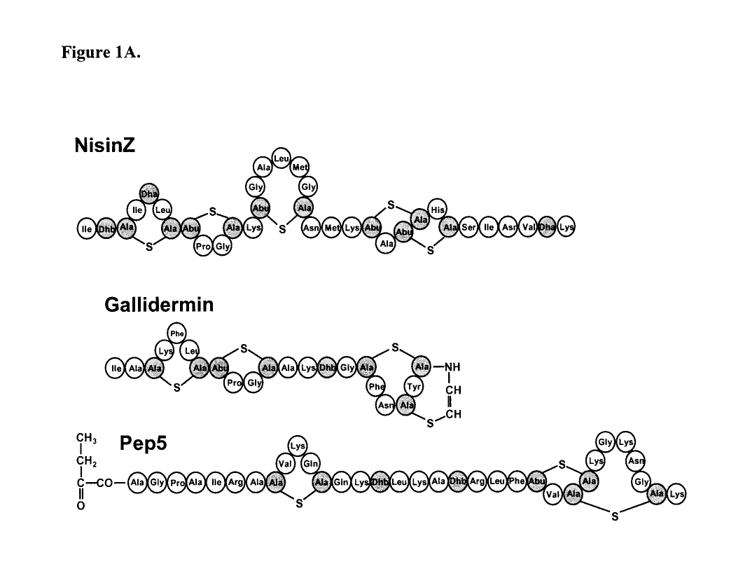

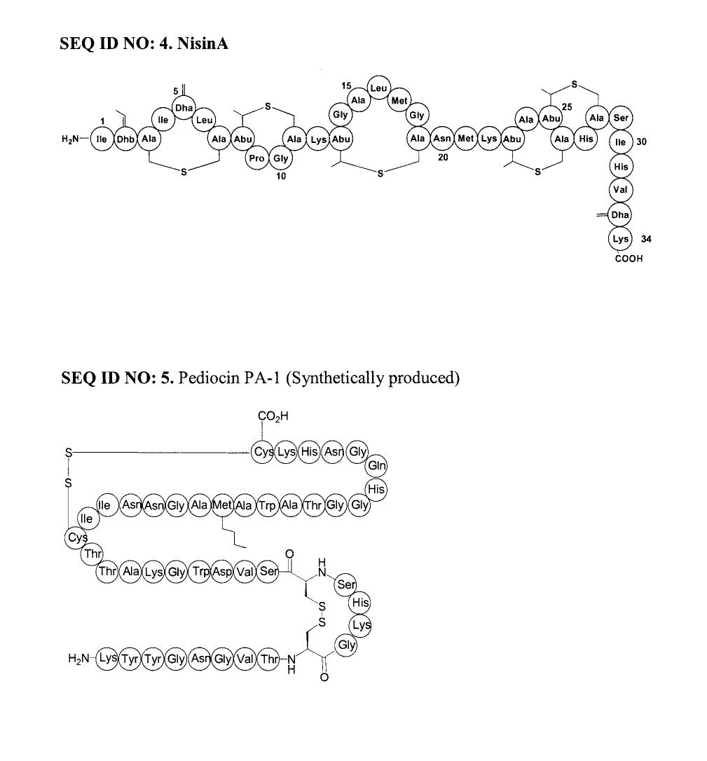

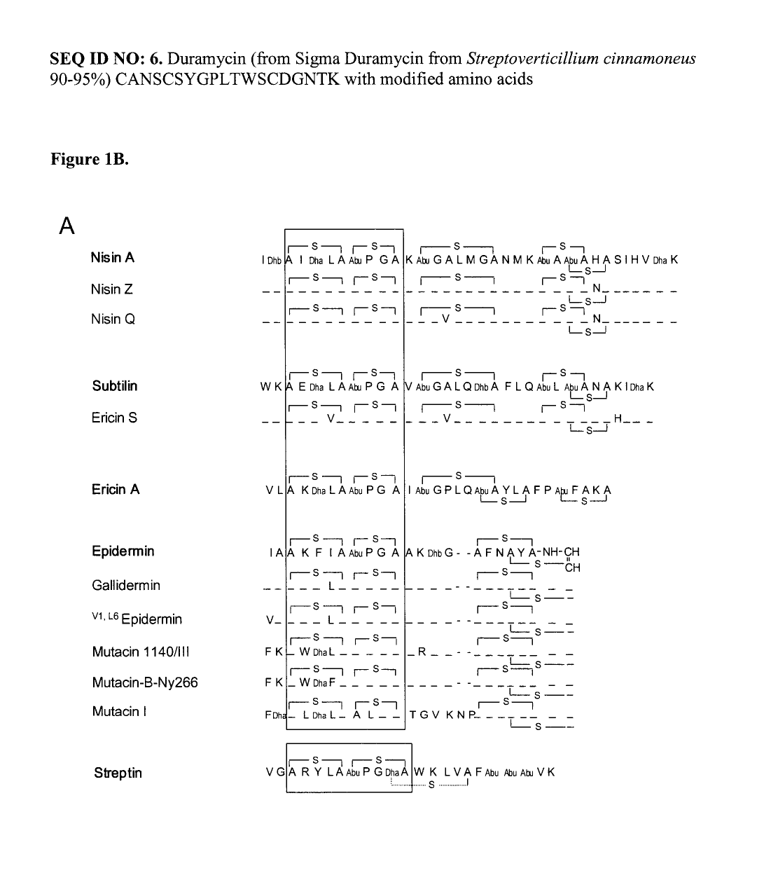

The invention provides an isolated peptide with immunomodulatory activity. Exemplary peptides of the invention have an amino acid sequence including those listed in FIG. 1A, and conservative variations thereof, wherein the peptides have immunomodulatory (chemokine-inducing) activity. The peptides of the invention include SEQ ID NOS:1-6, as well as the broader groups of peptides having hydrophilic and hydrophobic substitutions, and conservative variations thereof and other known lantibiotic peptides (FIG. 1B and Table 2).

“Isolated” when used in reference to a peptide, refers to a peptide substantially free of proteins, lipids, nucleic acids, for example, with which it might be naturally associated. Those of skill in the art can identify natural peptides with minor amino acid substitutions to achieve peptides with substantially equivalent immunomodulatory activities. Such modifications can be deliberate, as by site-directed mutagenesis, or can be spontaneous. All of the peptides produced by these modifications are included herein as long as the biological activity of the original peptide still exists.

Further, deletion of one or more amino acids can also result in a modification of the structure of the resultant molecule without significantly altering its biological activity. This can lead to the development of a smaller active molecule that would also have utility. For example, amino or carboxy terminal amino acids that can not be required for biological activity of the particular peptide can be removed. Peptides of the invention include any analog, homolog, mutant, isomer or derivative of the peptides disclosed in the present invention, so long as the bioactivity as described herein remains. In addition, C-terminal derivatives can be easily produced, such as C-terminal methyl esters and C-terminal amidates, in order to increase the activity of a peptide of the invention. The peptide can be synthesized such that the sequence is reversed whereby the last amino acid in the sequence becomes the first amino acid, and the penultimate amino acid becomes the second amino acid, and so on. It is well known that such reversed peptides usually have similar antimicrobial activities to the original sequence.

In certain aspects, the peptides of the invention include peptide analogs and peptide mimetics. Indeed, the peptides of the invention include peptides having any of a variety of different modifications, including those described herein.

Peptide analogs of the invention are generally natural fermentation products, including, e.g., any of the particular peptides described herein, such as any of the following sequences disclosed in the tables. The present invention clearly establishes that these peptides in their entirety and derivatives created by modifying any side chains of the constituent amino acids have the ability to modulate immune responses in human cells. The present invention further encompasses bacterial derived polypeptides up to about 50 amino acids in length that include the amino acid sequences and functional variants or peptide mimetics of the sequences described herein.

To improve or alter the characteristics of polypeptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the polypeptides of the present invention can be produced as multimers including dimers, trimers and tetramers. Multimerization can be facilitated by linkers, introduction of cysteines to permit creation of interchain disulphide bonds, or recombinantly though heterologous polypeptides such as Fc regions.

It is known in the art that one or more amino acids can be deleted from the N-terminus or C-terminus without substantial loss of biological function. See, e.g., Ron et al., Biol. Chem. 268: 2984-2988, 1993. Accordingly, the present invention provides polypeptides having one or more residues deleted from the amino terminus. Similarly, many examples of biologically functional C-terminal deletion mutants are known (see, e.g., Dobeli et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial chaperone polypeptide activity. Such mutants include deletions, insertions, inversions, repeats, and substitutions selected according to general rules known in the art so as to have little effect on activity.

There are two main approaches for studying the tolerance of an amino acid sequence to change, see, Bowie et al., Science 247: 1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the polypeptide of the present invention can be, for example: (i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the polypeptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, polypeptides of the present invention can include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific aspect of the invention includes polypeptides that include one or more amino acid substitutions that mimic modified amino acids at positions where amino acids that are capable of being modified are normally positioned. Further included are polypeptides where any subset of modifiable amino acids is substituted. For example, a polypeptide that includes three serine residues can be substituted at any one, any two, or all three of said serines. Furthermore, any polypeptide amino acid capable of being modified can be excluded from substitution with a modification-mimicking amino acid.

The present invention is further directed to fragments of the polypeptides of the present invention. More specifically, the present invention embodies purified, isolated, and recombinant polypeptides comprising at least any one integer between 6 and 504 (or the length of the polypeptides amino acid residues minus 1 if the length is less than 1000) of consecutive amino acid residues. Preferably, the fragments are at least 6, preferably at least 8 to 10, more preferably 12, 15, 20, 25, 30, 35, 40, 50 or more consecutive amino acids of a polypeptide of the present invention.

The present invention also provides for the exclusion of any species of polypeptide fragments of the present invention specified by 5′ and 3′ positions or sub-genuses of polypeptides specified by size in amino acids as described above. Any number of fragments specified by 5′ and 3′ positions or by size in amino acids, as described above, can be excluded.

In addition, it should be understood that in certain aspects, the peptides of the present invention include two or more modifications, including, but not limited to those described herein. By taking into the account the features of the peptide drugs on the market or under current development, it is clear that most of the peptides successfully stabilized against proteolysis consist of a mixture of several types of the above described modifications. This conclusion is understood in the light of the knowledge that many different enzymes are implicated in peptide degradation.

### C. Peptides, Peptide Variants, and Peptide Mimetics

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid mimetics refers to chemical compounds that have a structure that is different from the general chemical structure of an amino acid, but which functions in a manner similar to a naturally occurring amino acid. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings. Other modified amino acids are included in Table 2.

“Peptide” as used herein includes peptides that are conservative variations of those peptides specifically exemplified herein. “Conservative variation” as used herein denotes the replacement of an amino acid residue by another, biologically similar residue. Examples of conservative variations include, but are not limited to, the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acids, or glutamine for asparagine, and the like. Neutral hydrophilic amino acids that can be substituted for one another include asparagine, glutamine, serine and threonine. The term “conservative variation” also includes the use of a substituted amino acid in place of an unsubstituted parent amino acid provided that antibodies raised to the substituted polypeptide also immunoreact with the unsubstituted polypeptide. Such conservative substitutions are within the definition of the classes of the peptides of the invention. “Cationic” as is used to refer to any peptide that possesses sufficient positively charged amino acids to have a pI (isoelectric point) greater than about 9.0.

The biological activity of the peptides can be determined by standard methods known to those of skill in the art, such as the chemokine induction method referred to below.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions so long as such substitutions do not also substantially alter the mimetic's structure and/or activity. As with polypeptides of the invention that are conservative variants, routine experimentation will determine whether a mimetic is within the scope of the invention, i.e., that its structure and/or function is not substantially altered. Thus, a mimetic composition is within the scope of the invention if, when administered to or expressed in a cell, e.g., a polypeptide fragment of an antimicrobial protein having antimicrobial activity.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropyl-carbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola 1983. in Chemistry and Biochemistry of Amino Acids, Peptides and Proteins, Vol. 7, pp 267-357, “Peptide Backbone Modifications,” Marcell Dekker, NY).

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3 (4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, or citrulline. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Histidine residue mimetics can be generated by reacting histidyl with, e.g., diethylprocarbonate or para-bromophenacyl bromide. Other mimetics include, e.g., those generated by hydroxylation of lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an antimicrobial polypeptide having antimicrobial activity of the invention, resulting in modification of the structure of the polypeptide, without significantly altering its biological activity. For example, amino- or carboxyl-terminal, or internal, amino acids that are not required for antimicrobial activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

Peptides of the invention can be synthesized by such commonly used methods as t-BOC or FMOC protection of alpha-amino groups. Both methods involve stepwise syntheses whereby a single amino acid is added at each step starting from the C terminus of the peptide (See, Coligan, et al., Current Protocols in Immunology, Wiley Interscience, 1991, Unit 9). Peptides of the invention can also be synthesized by the well known solid phase peptide synthesis methods described in Merrifield, J. Am. Chem. Soc. 85:2149, (1962), and Stewart and Young, Solid Phase Peptides Synthesis, (Freeman, San Francisco, 1969, pp. 27-62), using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with 1% acetic acid solution which is then lyophilized to yield the crude material. This can normally be purified by such techniques as gel filtration on Sephadex G-15 using 5% acetic acid as a solvent. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Analogs, polypeptide fragments of immunomodulatory peptides, are generally designed and produced by chemical modifications of a lead peptide, including, e.g., any of the particular peptides described herein, such as any of the sequences including SEQ ID NOS:1-6.

The terms “identical” or percent “identity”, in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Nat'l. Acad. Sci. USA 85: 2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement)).

Programs for searching for alignments are well known in the art, e.g., BLAST and the like. For example, if the target species is human, a source of such amino acid sequences or gene sequences (germline or rearranged antibody sequences) can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain aspects, an acceptable range of homology is greater than about 50%. It should be understood that target species can be other than human.

A preferred example of algorithm that is suitable for determining percent sequence identity and sequence similarity are the BLAST and BLAST 2.0 algorithms, which are described in Altschul et al., Nuc. Acids Res. 25: 3389-3402, 1977 and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89: 10915, 1989) alignments (B) of 50, expectation (E) of 10, M=5, N=−4, and a comparison of both strands.

### D. Polypeptides and Functional Variants Thereof

“Polypeptide” includes proteins, fusion proteins, oligopeptides and polypeptide derivatives, with the exception that peptidomimetics are considered to be small molecules herein.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of recombinant protein that has an amino acid sequence that results from the linkage of the amino acid sequences of two or more normally separate polypeptides.

A “protein fragment” is a proteolytic fragment of a larger polypeptide, which can be a protein or a fusion protein. A proteolytic fragment can be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” or “peptide” is a polypeptide having a short amino acid sequence (i.e., 2 to about 200 amino acids). An oligopeptide is generally prepared by chemical synthesis.

Although oligopeptides and protein fragments can be otherwise prepared, it is possible to use recombinant DNA technology and/or in vitro biochemical manipulations. For example, a nucleic acid encoding an amino acid sequence can be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabeled amino acid(s). In this manner, polypeptides encoded by a pre-selected exogenous nucleic acid are radiolabeled. Although other proteins are present in the reaction mix, the pre-selected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidomimetics.

The polypeptides of this invention, including the analogs and other modified variants, can generally be prepared following known techniques. Preferably, synthetic production of the polypeptide of the invention can be according to the solid phase synthetic method. For example, the solid phase synthesis is well understood and is a common method for preparation of polypeptides, as are a variety of modifications of that technique. Merrifield, J. Am. Chem. Soc. 85: 2149, 1964; Stewart and Young, Solid Phase Polypeptide Synthesis Pierce Chemical Company, Rockford, Ill., 1984; Bodansky and Bodanszky, The Practice of polypeptide Synthesis, Springer-Verlag, New York, 1984; Atherton and Sheppard, Solid Phase polypeptide Synthesis: A Practical Approach, IRL Press, New York, 1989). See, also, the specific method described in Example 1 below.

Alternatively, polypeptides of this invention can be prepared in recombinant systems using polynucleotide sequences encoding the polypeptides.

A “variant” or “functional variant” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention can contain more than one of the following modifications within the same polypeptide. Preferred polypeptide derivatives retain a desirable attribute, which can be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

A polypeptide having an amino acid sequence identical to that found in a protein prepared from a natural source is a “wildtype” polypeptide. Functional variants of polypeptides can be prepared by chemical synthesis, including without limitation combinatorial synthesis.

Functional variants of polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences can comprise substitutions, deletions and additions of amino acids, with the proviso that such amino acids are naturally occurring amino acids.

Thus, subjecting a nucleic acid that encodes a polypeptide to mutagenesis is one technique that can be used to prepare Functional variants of polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis can be randomly targeted (i.e., mutations can occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids can be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences can be prepared by directed mutagenesis.

As contemplated by this invention, “polypeptide” includes those having one or more chemical modification relative to another polypeptide, i.e., chemically modified polypeptides. The polypeptide from which a chemically modified polypeptide is derived can be a wildtype protein, a functional variant protein or a functional variant polypeptide, or polypeptide fragments thereof an antibody or other polypeptide ligand according to the invention including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharmaceutical Research 10: 1268-1273, 1993). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. 1993, cited above).

Different host cells will contain different post-translational modification mechanisms that can provide particular types of post-translational modification of a fusion protein if the amino acid sequences, required for such modifications, is present in the fusion protein. A large number (about 100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and can influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation can vary depending on which host cells are used to produce the fusion protein.

Another type of post-translation modification is the phosphorylation of a free hydroxyl group of the side chain of one or more Ser, Thr or Tyr residues, Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which can have a different chemical version of the methionine residue encoded by a start codon, and these will result in amino termini with different chemical modifications.

For example, many or most bacterial proteins are synthesized with an amino terminal amino acid that is a modified form of methionine, i.e., N-formyl-methionine (fMet). Although the statement is often made that all bacterial proteins are synthesized with an fMet initiator amino acid; although this can be true for E. coli, recent studies have shown that it is not true in the case of other bacteria such as Pseudomonas aeruginosa (Newton et al., J. Biol. Chem. 274: 22143-22146, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.). E. coli mutants that lack the enzymes (such as, e.g., formylase) that catalyze such post-translational modifications will produce proteins having an amino terminal fMet residue (Guillon et al., J. Bacteriol. 174: 4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23: 263-267, 1998; and Driessen et al., CRC Crit. Rev. Biochem. 18: 281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50: 333-344, 1988, and Bradbury et al. Lung Cancer 14: 239-251, 1996. About 50% of known endocrine and neuroendocrine peptide hormones are alpha-amidated (Treston et al., Cell Growth Differ. 4: 911-920, 1993). In most cases, carboxy alpha-amidation is required to activate these peptide hormones.

### E. Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides can exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays, 16: 683-687, 1994; Cohen and Shatzmiller, J. Mol. Graph., 11: 166-173, 1993; Wiley and Rich, Med. Res. Rev., 13: 327-384, 1993; Moore, Trends Pharmacological Science, 15: 124-129, 1994; Hruby, Biopolymers, 33: 1073-1082, 1993; Bugg et al., Scientific American, 269: 92-98, 1993, all incorporated herein by reference).

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., Int. J. Polypeptide Protein Res. 41: 181-184, 1993, incorporated herein by reference). Thus, the amino acid sequences of these compounds can be identical to the sequences of their parent L-amino acid polypeptides, except that one or more of the peptide bonds are replaced by an isosteric pseudopeptide bond. Preferably the most N-terminal peptide bond is substituted, since such a substitution would confer resistance to proteolysis by exopeptidases acting on the N-terminus.

To confer resistance to proteolysis, peptide bonds can also be substituted by retro-inverso pseudopeptide bonds (Dalpozzo, et al., Int. J. Polypeptide Protein Res. 41: 561-566, incorporated herein by reference). According to this modification, the amino acid sequences of the compounds can be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid derivatives of polypeptides represent another form of modified polypeptides that retain the important structural determinants for biological activity, yet eliminate the peptide bonds, thereby conferring resistance to proteolysis (Simon, et al., Proc. Natl. Acad. Sci. USA, 89: 9367-9371, 1992, and incorporated herein by reference). Peptoids are oligomers of N-substituted glycines. A number of N-alkyl groups have been described, each corresponding to the side chain of a natural amino acid.

### F. Polynucleotides

The invention includes polynucleotides encoding peptides of the invention. Exemplary polynucleotides encode peptides including those listed in Table 1, and analogs, derivatives, amidated variations and conservative variations thereof, wherein the peptides have antimicrobial activity. The peptides of the invention include SEQ ID NOS:1-6, as well as the broader groups of peptides having hydrophilic and hydrophobic substitutions, and conservative variations thereof.

To measure the transcription level (and thereby the expression level) of a gene or genes, a nucleic acid sample comprising mRNA transcript(s) of the gene or genes, or nucleic acids derived from the mRNA transcript(s) is provided. A nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include mRNA transcripts of the gene or genes, cDNA reverse transcribed from the mRNA, cRNA transcribed from the cDNA, DNA amplified from the genes, RNA transcribed from amplified DNA, and the like.

In some methods, a nucleic acid sample is the total mRNA isolated from a biological sample. The term “biological sample”, as used herein, refers to a sample obtained from an organism or from components (e.g., cells) or an organism. The sample can be of any biological tissue or fluid. Frequently the sample is from a patient. Such samples include sputum, blood, blood cells (e.g., white cells), tissue or fine needle biopsy samples, urine, peritoneal fluid, and fleural fluid, or cells therefrom. Biological samples can also include sections of tissues such as frozen sections taken for histological purposes. Often two samples are provided for purposes of comparison. The samples can be, for example, from different cell or tissue types, from different species, from different individuals in the same species or from the same original sample subjected to two different treatments (e.g., drug-treated and control).

“Isolated” when used in reference to a polynucleotide, refers to a polynucleotide substantially free of proteins, lipids, nucleic acids, for example, with which it is naturally associated. As used herein, “polynucleotide” refers to a polymer of deoxyribonucleotides or ribonucleotides, in the form of a separate fragment or as a component of a larger construct. DNA encoding a peptide of the invention can be assembled from cDNA fragments or from oligonucleotides which provide a synthetic gene which is capable of being expressed in a recombinant transcriptional unit. Polynucleotide sequences of the invention include DNA, RNA and cDNA sequences. A polynucleotide sequence can be deduced from the genetic code, however, the degeneracy of the code must be taken into account. Polynucleotides of the invention include sequences which are degenerate as a result of the genetic code. Such polynucleotides are useful for the recombinant production of large quantities of a peptide of interest, such as the peptide of SEQ ID NOS:1-6.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

Alternatively, these nucleic acids can be synthesized in vitro by well-known chemical synthesis techniques, as described in, e.g., Adams, J. Am. Chem. Soc. 105: 661, 1983; Belousov, Nucleic Acids Res. 25: 3440-3444, 1997; Frenkel, Free Radic. Biol. Med. 19: 373-380, 1995; Blommers, Biochemistry 33: 7886-7896, 1994; Narang, Meth. Enzymol. 68: 90, 1979; Brown Meth. Enzymol. 68: 109, 1979; Beaucage, Tetra. Lett. 22: 1859, 1981; U.S. Pat. No. 4,458,066.

In accordance with the present invention, there can be employed conventional molecular biology, microbiology, immunology, and recombinant DNA techniques within the skill of the art. Such techniques are explained fully in the literature. Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., See, for example, Sambrook, Fitsch & Maniatis, 1989, Molecular Cloning: A Laboratory Manual, 2^{nd}, Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (Hames, B. D. & S. J. Higgins, eds. 1984); Animal Cell Culture (R. I. Freshney, ed. 1986); Immobilized Cells and Enzymes (IRL Press, 1986); B. E. Perbal, 1984, A Practical Guide to Molecular Cloning; F. M. Ausubel et al. (eds.), Current Protocols in Molecular Biology, 1997, John Wiley & Sons, Inc., N. C. Dracopoli et al. (eds.), Current Protocols in Human Genetics, 1997, John Wiley & Sons, Inc., A. D. Baxevanis et al. (eds.), Current Protocols in Bioinformatics, 1992, John Wiley & Sons, Inc.; Laboratory Techniques In Biochemistry And Molecular Biology: Hybridization With Nucleic Acid Probes, Part I. Theory and Nucleic Acid Preparation, Tijssen, ed. Elsevier, N.Y., 1993 (these references are herein incorporated by reference in their entirety for all purposes).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassay (RIAs), enzyme-linked immunosorbent assays (ELISAs), immunofluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat. Genet. 15: 333-335, 1997; yeast artificial chromosomes (YAC); bacterial artificial chromosomes (BAC); P1 artificial chromosomes, see, e.g., Woon, Genomics 50: 306-316, 1998; P1-derived vectors (PACs), see, e.g., Kern, Biotechniques 23:120-124, 1997; cosmids, recombinant viruses, phages or plasmids.

The invention provides fusion proteins and nucleic acids encoding them. A gene product or polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego, Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif 12: 404-414, 1998). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell. Biol. 12: 441-53, 1993.

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The invention provides expression vectors and cloning vehicles comprising nucleic acids of the invention, e.g., sequences encoding the proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). See, for example, U.S. Pat. No. 5,707,855. Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature 328: 731, 1987; Schneider, Protein Expr. Purif. 6435: 10, 1995; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors can also be employed which exploit advantageous merits of each of the parent vector properties (See e.g., Feng, Nature Biotechnology 15: 866-870, 1997). Such viral genomes can be modified by recombinant DNA techniques to include the nucleic acids of the invention; and can be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher, J. Virol. 66: 2731-2739, 1992; Johann, J. Virol. 66: 1635-1640, 1992). Adeno-associated virus (AAV)-based vectors can be used to transfect cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther. 3: 957-964, 1996.

“Expression cassette” as used herein refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination. Thus, expression cassettes also include plasmids, expression vectors, recombinant viruses, any form of recombinant “naked DNA” vector, and the like.

“Vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The invention also provides a transformed cell comprising a nucleic acid sequence of the invention, e.g., a sequence encoding a polypeptide of the invention, or a vector of the invention. The host cell can be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the immunomodulatory bacteriocin or lantibiotic protein or related protein sequences, or subsequences thereof.

Amplification methods include, e.g., polymerase chain reaction, PCR (PCR Protocols, A Guide To Methods And Applications, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4: 560, 1989; Landegren, Science 241: 1077, 1988; Barringer, Gene 89: 117, 1990); transcription amplification (see, e.g., Kwoh, Proc. Natl. Acad. Sci. USA 86: 1173, 1989); and, self-sustained sequence replication (see, e.g., Guatelli, Proc. Natl. Acad. Sci. USA 87: 1874, 1990); Q Beta replicase amplification (see, e.g., Smith, J. Clin. Microbiol. 35: 1477-1491, 1997), automated Q-beta replicase amplification assay (see, e.g., Burg, Mol. Cell. Probes 10: 257-271, 1996) and other RNA polymerase mediated techniques (e.g., NASBA, Cangene, Mississauga, Ontario); see also Berger, Methods Enzymol. 152: 307-316, 1987; Sambrook; Ausubel; U.S. Pat. Nos. 4,683,195 and 4,683,202; Sooknanan, Biotechnology 13: 563-564, 1995.

The invention provides isolated or recombinant nucleic acids that hybridize under stringent conditions to an exemplary sequence of the invention, e.g., a sequence or related sequence, or the complement of any thereof, or a nucleic acid that encodes a polypeptide of the invention (See also SEQ ID NO:1-6). In alternative aspects, the stringent conditions are highly stringent conditions, medium stringent conditions or low stringent conditions, as known in the art and as described herein. These methods can be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention by its ability to hybridize under stringent conditions to a nucleic acid otherwise determined to be within the scope of the invention (such as the exemplary sequences described herein).

“Stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in, e.g., Sambrook, ed., 1989; Ausubel, ed. 1997; Tijssen, ed., 1993, supra).

Generally, stringent conditions are selected to be about 5-10° C. lower than the thermal melting point I for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide as described in Sambrook (cited below). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media can be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

The polynucleotide sequence encoding the peptide used according to the method of the invention can be isolated from an organism or synthesized in the laboratory. Specific DNA sequences encoding the peptide of interest can be obtained by: 1) isolation of a double-stranded DNA sequence from the genomic DNA; 2) chemical manufacture of a DNA sequence to provide the necessary codons for the peptide of interest; and 3) in vitro synthesis of a double-stranded DNA sequence by reverse transcription of mRNA isolated from a donor cell. In the latter case, a double-stranded DNA complement of mRNA is eventually formed that is generally referred to as cDNA.

The synthesis of DNA sequences is frequently the method of choice when the entire sequence of amino acid residues of the desired peptide product is known. In the present invention, the synthesis of a DNA sequence has the advantage of allowing the incorporation of codons that are more likely to be recognized by a bacterial host, thereby permitting high level expression without difficulties in translation. In addition, virtually any peptide can be synthesized, including those encoding natural peptides, variants of the same, or synthetic peptides.

When the entire sequence of the desired peptide is not known, the direct synthesis of DNA sequences is not possible and the method of choice is the formation of cDNA sequences. Among the standard procedures for isolating cDNA sequences of interest is the formation of plasmid or phage containing cDNA libraries that are derived from reverse transcription of mRNA that is abundant in donor cells that have a high level of genetic expression. When used in combination with polymerase chain reaction technology, even rare expression products can be cloned. In those cases where significant portions of the amino acid sequence of the peptide are known, the production of labeled single or double-stranded DNA or RNA probe sequences duplicating a sequence putatively present in the target cDNA can be employed in DNA/DNA hybridization procedures which are carried out on cloned copies of the cDNA which have been denatured into a single stranded form (Jay, et al., Nuc. Acid Res., 11:2325, 1983).

### G. Methods of Use

### Immunomodulatory

The present invention provides novel cationic bacteriocin peptides and lantibiotics which have ability to modulate (e.g., up- and/or down regulate) polypeptide expression, thereby regulating sepsis and inflammatory responses and/or innate immunity.

“Modulator” includes inhibitors and activators. Inhibitors are agents that, e.g., bind to, partially or totally block stimulation, decrease, prevent, delay activation, inactivate, desensitize, or down regulate activity, e.g., antagonists. Activators are agents that, e.g., bind to, stimulate, increase, open, activate, facilitate, enhance activation, sensitize or up regulate activity, e.g., agonists. Modulators include agents that, e.g., alter the interaction of receptor with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring receptor ligands, e.g., with altered activity, as well as naturally occurring and synthetic ligands, antagonists, agonists, small chemical molecules and the like.

“Cell-based assays” for inhibitors and activators include, e.g., applying putative modulator compounds to a cell expressing a receptor, e.g., surface receptors, and then determining the functional effects on receptor signaling, as described herein. Cell-based assays or include, but are not limited to, in vivo tissue or cell samples from a mammalian subject or in vitro cell-based assays comprising a receptor that are treated with a potential activator, inhibitor, or modulator are compared to control samples without the inhibitor, activator, or modulator to examine the extent of inhibition. These assays include binding assays, for example, radioligand or fluorescent ligand binding assays to cells, plasma membranes, detergent-solubilized plasma membrane proteins, immobilized collagen Control samples (untreated with inhibitors) can be assigned a relative activity value of 100%. Inhibition of a receptor is achieved when the receptor activity value relative to the control is about 80%, optionally 50% or 25-0%. Activation of a receptor is achieved when the receptor activity value relative to the control is 110%, optionally 150%, optionally 200-500%, or 1000-3000% higher.

“Innate immunity” as used herein refers to the natural ability of an organism to defend itself against invasions by pathogens. Pathogens or microbes as used herein, can include, but are not limited to bacteria, fungi, parasites, and viruses. Innate immunity is contrasted with acquired/adaptive immunity in which the organism develops a defensive mechanism based substantially on antibodies and/or immune lymphocytes that is characterized by specificity, amplifiability and self vs. non-self discrimination. With innate immunity, broad, nonspecific immunity is provided and there is no immunologic memory of prior exposure. The hallmarks of innate immunity are effectiveness against a broad variety of potential pathogens, independence of prior exposure to a pathogen, and immediate effectiveness (in contrast to the specific immune response which takes days to weeks to be elicited). In addition, innate immunity includes immune responses that affect other diseases, such as cancer, inflammatory diseases, multiple sclerosis, various viral infections, and the like.

In innate immunity, the immune response is not dependent upon antigens. The innate immunity process can include the production of secretory molecules and cellular components as set forth above. In innate immunity, the pathogens are recognized by receptors (for example, Toll-like receptors) that have broad specificity, are capable of recognizing many pathogens, and are encoded in the germline. These Toll-like receptors have broad specificity and are capable of recognizing many pathogens. When cationic peptides are present in the immune response, they aid in the host response to pathogens. This change in the immune response induces the release of chemokines, which promote the recruitment of immune cells to the site of infection.

“Adjuvanticity” as used herein is the ability to modify the immune response (e.g., the peptides of the present invention modify the immune response which leads to the promotion of a subsequent antibody response).

Chemokines, or chemoattractant cytokines, are a subgroup of immune factors that mediate chemotactic and other pro-inflammatory phenomena (See, Schall, 1991, Cytokine 3:165-183). Chemokines are small molecules of approximately 70-80 residues in length and can generally be divided into two subgroups, α which have two N-terminal cysteines separated by a single amino acid (C×C) and β which have two adjacent cysteines at the N terminus (CC). RANTES, MIP-1α and MIP-1β are members of the β subgroup (reviewed by Horuk, R., 1994, Trends Pharmacol. Sci, 15:159-165; Murphy, P. M., 1994, Annu. Rev. Immunol., 12:593-633). The amino terminus of the β chemokines RANTES, MCP-1, and MCP-3 have been implicated in the mediation of cell migration and inflammation induced by these chemokines. This involvement is suggested by the observation that the deletion of the amino terminal 8 residues of MCP-1, amino terminal 9 residues of MCP-3, and amino terminal 8 residues of RANTES and the addition of a methionine to the amino terminus of RANTES, antagonize the chemotaxis, calcium mobilization and/or enzyme release stimulated by their native counterparts (Gong et al., 1996 J. Biol. Chem. 271:10521-10527; Proudfoot et al., 1996 J. Biol. Chem. 271:2599-2603). Additionally, α chemokine-like chemotactic activity has been introduced into MCP-1 via a double mutation of Tyr 28 and Arg 30 to leucine and valine, respectively, indicating that internal regions of this protein also play a role in regulating chemotactic activity (Beall et al., 1992, J. Biol. Chem. 267:3455-3459).

The monomeric forms of all chemokines characterized thus far share significant structural homology, although the quaternary structures of α and β groups are distinct. While the monomeric structures of the β and α chemokines are very similar, the dimeric structures of the two groups are completely different. An additional chemokine, lymphotactin, which has only one N terminal cysteine has also been identified and can represent an additional subgroup (y) of chemokines (Yoshida et al., 1995, FEBS Lett. 360:155-159; and Kelner et al., 1994, Science 266:1395-1399).

Receptors for chemokines belong to the large family of G-protein coupled, 7 transmembrane domain receptors (GCR's) (See, reviews by Horuk, R., 1994, Trends Pharmacol. Sci. 15:159-165; and Murphy, P. M., 1994, Annu. Rev. Immunol. 12:593-633). Competition binding and cross-desensitization studies have shown that chemokine receptors exhibit considerable promiscuity in ligand binding. Examples demonstrating the promiscuity among β chemokine receptors include: CC CKR-1, which binds RANTES and MIP-1α (Neote et al., 1993, Cell 72: 415-425), CC CKR-4, which binds RANTES, MIP-1α, and MCP-1 (Power et al., 1995, J. Biol. Chem. 270:19495-19500), and CC CKR-5, which binds RANTES, MIP-1α, and MIP-1β (Alkhatib et al., 1996, Science, in press and Dragic et al., 1996, Nature 381:667-674). Erythrocytes possess a receptor (known as the Duffy antigen) which binds both α and β chemokines (Horuk et al., 1994, J. Biol. Chem. 269:17730-17733; Neote et al., 1994, Blood 84:44-52; and Neote et al., 1993, J. Biol. Chem. 268:12247-12249). Thus the sequence and structural homologies evident among chemokines and their receptors allows some overlap in receptor-ligand interactions.

In one aspect, the present invention provides the use of compounds including peptides of the invention to reduce sepsis and inflammatory responses by acting directly on host cells. In this aspect, a method of identification of a polynucleotide or polynucleotides that are regulated by one or more sepsis or inflammatory inducing agents is provided, where the regulation is altered by a cationic peptide. Such sepsis or inflammatory inducing agents include, but are not limited to endotoxic lipopolysaccharide (LPS), lipoteichoic acid (LTA) and/or CpG DNA or intact bacteria or other bacterial components. The identification is performed by contacting the host cell with the sepsis or inflammatory inducing agents and further contacting with a cationic peptide either simultaneously or immediately after. The expression of the polynucleotide or polypeptide in the presence and absence of the cationic peptide is observed and a change in expression is indicative of a polynucleotide or polypeptide or pattern of polynucleotides or polypeptides that is regulated by a sepsis or inflammatory inducing agent and inhibited by a cationic peptide. In another aspect, the invention provides a polynucleotide identified by the method.

“Test compound” refers to a nucleic acid, DNA, RNA, protein, polypeptide, or small chemical entity that is determined to effect an increase or decrease in a gene expression. The test compound can be an antisense RNA, ribozyme, polypeptide, or small molecular chemical entity. The term “test compound” can be any small chemical compound, or a biological entity, such as a protein, sugar, nucleic acid or lipid. Typically, test compounds will be small chemical molecules and polypeptides (described further below).

“Contacting” refers to mixing a test compound or agent in a soluble form into an assay system, for example, a cell-based assay system, such that an effect, for example, modulating an innate immune response, can be measured.

Candidate agents or test compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and can be used to produce combinatorial libraries. Known pharmacological agents can be subjected to directed or random chemical modifications, such as acylation, alkylation, esterification, amidification, and the like to produce structural analogs. Candidate agents or test compounds are also found among biomolecules including, but not limited to: peptides, peptidiomimetics, saccharides, fatty acids, steroids, purines, pyrimidines, polypeptides, polynucleotides, chemical compounds, derivatives, structural analogs or combinations thereof.

Generally, in the methods of the invention, a cationic lantibiotic or bacteriocin peptide is utilized to detect and locate a polynucleotide or polypeptide that is essential in the process of sepsis or inflammation. Once identified, a pattern of polynucleotide or polypeptide expression can be obtained by observing the expression in the presence and absence of the cationic peptide. The pattern obtained in the presence of the cationic peptide is then useful in identifying additional compounds that can inhibit expression of the polynucleotide and therefore block sepsis or inflammation. It is well known to one of skill in the art that non-peptidic chemicals and peptidomimetics can mimic the ability of peptides to bind to receptors and enzyme binding sites and thus can be used to block or stimulate biological reactions. Where an additional compound of interest provides a pattern of polynucleotide or polypeptide expression similar to that of the expression in the presence of a cationic peptide, that compound is also useful in the modulation of sepsis or an innate immune response. In this manner, the cationic peptides of the invention, which are known inhibitors of sepsis and inflammation and enhancers of innate immunity are useful as tools in the identification of additional compounds that inhibit sepsis and inflammation and enhance innate immunity.

As can be seen in Example 2 below, peptides of the invention have an ability to alter the expression of polynucleotides or polypeptides regulated by LPS, particularly the quintessential pro-inflammatory cytokine TNFα. High levels of endotoxin in the blood are responsible for many of the symptoms seen during a serious infection or inflammation such as fever and an elevated white blood cell count, and many of these effects reflect or are caused by high levels of induced TNFα. Endotoxin (also called lipopolysaccharide) is a component of the cell wall of Gram-negative bacteria and is a potent trigger of the pathophysiology of sepsis. The basic mechanisms of inflammation and sepsis are related.

In another aspect, the invention identifies agents that enhance innate immunity. Human cells that contain a polynucleotide or polynucleotides that encode a polypeptide or polypeptides involved in innate immunity are contacted with an agent of interest. Expression of the polynucleotide is determined, both in the presence and absence of the agent. The expression is compared and of the specific modulation of expression was indicative of an enhancement of innate immunity. In another aspect, the agent does not stimulate a septic reaction as revealed by the lack of upregulation of the pro-inflammatory cytokine TNF-α. In still another aspect the agent reduces or blocks the inflammatory or septic response.

In another aspect, a method for identifying a compound which modulates an innate immune response is provided comprising: (a) providing a cell-based assay system comprising a cell containing a gene that encodes a polypeptide involved in innate immunity and protection against infection, expression of the gene being modulated during an innate immune response; (b) contacting the cell with a test compound; and (c) measuring expression of the gene in the assay system, wherein a difference in expression in the presence of the compound relative to expression in the absence of the compound is indicative of modulation.

In some aspects, the compound is an agonist of an innate immune response. In other aspects, the compound is an antagonist of an innate immune response. In some aspects, the compound is an inhibitor of an innate immune response. In other aspects, the compound is an activator of an innate immune response. In some aspects, the test compound is an organic molecule, a natural product, a peptide, an oligosaccharide, a nucleic acid, a lipid, an antibody, or binding fragment thereof. In other aspects, the test compound is from a library of compounds. In some aspects, the library is a random peptide library, a combinatorial library, an oligosaccharide library or a phage display library.

In another aspect, the invention provides methods of direct polynucleotide or polypeptide regulation by cationic peptides and the use of compounds including cationic peptides to stimulate elements of innate immunity. In this aspect, the invention provides a method of identification of a pattern of polynucleotide or polypeptide expression for identification of a compound that enhances innate immunity. In the method of the invention, an initial detection of a pattern of polypeptide expression for cells contacted in the presence and absence of a cationic peptide is made. The pattern resulting from polypeptide expression in the presence of the peptide represents stimulation of innate immunity. A pattern of polypeptide expression is then detected in the presence of a test compound, where a resulting pattern with the test compound that is similar to the pattern observed in the presence of the cationic peptide is indicative of a compound that enhances innate immunity. In another aspect, the invention provides compounds that are identified in the above methods. In another aspect, the compound of the invention stimulates chemokine expression. Chemokine or chemokine receptors can include, but are not limited to IL8, Gro-α, MCP-1, and MCP-3. In still another aspect, the compound is a peptide, peptidomimetic, chemical compound, or a nucleic acid molecule.

In another aspect, methods of selectively enhancing innate immunity are provided comprising contacting a cell containing a gene that encodes a polypeptide involved in innate immunity and protection against an infection with an isolated immunomodulatory bacteriocin or lantibiotic peptide with net cationic charge, wherein expression of the gene in the presence of the bacteriocin or lantibiotic peptide is modulated as compared with expression of the gene in the absence of the bacteriocin or lantibiotic peptide, and wherein the modulated expression results in enhancement of innate immunity.

In another aspect, methods of selectively suppressing a proinflammatory response are provided comprising contacting a cell containing a gene that encodes a polypeptide involved in inflammation and sepsis with an isolated immunomodulatory bacteriocin or lantibiotic peptide with net cationic charge, wherein the expression of the gene is modulated in the presence of the bacteriocin or lantibiotic peptide compared with expression in the absence of the bacteriocin or lantibiotic peptide, and wherein the modulated expression results in enhancement of innate immunity.

It is shown below, for example, in FIGS. 2-5, 7 and 8, that cationic peptides can alter the host response to the signaling molecules of infectious agents as well as modify the transcriptional responses of host cells, mainly by down-regulating the pro-inflammatory response and/or up-regulating the anti-inflammatory response. Example 1 shows that the cationic peptides can aid in the host response to pathogens by inducing the release of chemokines, which promote the recruitment of immune cells to the site of infection. Example 2 shows that the cationic peptides can selectively suppress the induction of the sepsis inducing cytokine TNFα in host cells.

In another aspect the stimulation of innate immunity by the peptide, particularly its ability to stimulate chemokines and thus the recruitment of immune cells, can lead to enhancement of an adaptive immune response to an antigen of interest, so-called adjuvant activity.

It is seen from the examples below that cationic peptides have a substantial influence on the host response to pathogens in that they assist in regulation of the host immune response by inducing selective pro-inflammatory responses that for example promote the recruitment of immune cells to the site of infection but not inducing potentially harmful pro-inflammatory cytokines. Sepsis appears to be caused in part by an overwhelming pro-inflammatory response to infectious agents. Peptides can aid the host in a “balanced” response to pathogens by inducing an anti-inflammatory response and suppressing certain potentially harmful pro-inflammatory responses. In addition they can assist in vaccine formulations due to their ability to promote adaptive immune responses through their chemokine activity.

### I. Treatment Regimes

The invention provides pharmaceutical compositions comprising one or a combination of antimicrobial peptides, for example, formulated together with a pharmaceutically acceptable carrier.

Some compositions include a combination of multiple (e.g., two or more) peptides of the invention. In one aspect, a pharmaceutical composition comprises an isolated immunomodulatory bacteriocin or lantibiotic peptide with net cationic charge together with a pharmaceutically acceptable carrier. In other aspects, the immunomodulatory bacteriocin or lantibiotic peptide is selected from the group consisting of SEQ ID NO: 1-6 or analogs, derivatives, amidated variations and conservative variations thereof. In other aspects, isolated polynucleotides encode these peptides. In other aspects, the invention further provides pharmaceutical compositions comprising polynucleotides of the invention together with a pharmaceutically acceptable carrier.

“Treating” or “treatment” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination. Accordingly, the term “treating” includes the administration of the compounds or agents of the present invention, i.e., novel cationic bacteriocin peptides and lantibiotics of the invention which have ability to modulate (e.g., up- and/or down regulate) polypeptide expression, thereby regulating sepsis and inflammatory responses and/or innate immunity. Accordingly, the term “treating” includes the administration of the compounds or agents of the present invention to prevent or delay, to alleviate, or to arrest or inhibit development of the symptoms or conditions associated with sepsis and inflammatory responses and/or innate immunity. The term “therapeutic effect” refers to the reduction, elimination, or prevention of the disease, symptoms of the disease, or side effects of the disease in the subject.

“Concomitant administration” of a known drug with a compound or agent of the present invention, i.e., novel cationic bacteriocin peptide(s) and lantibiotic(s) of the invention. means administration of the drug and the compound or compounds at such time that both the known drug and the compound or compounds will have a therapeutic effect or diagnostic effect. Such concomitant administration can involve concurrent (i.e., at the same time), prior, or subsequent administration of the drug with respect to the administration of a compound/agent or compounds/agents of the present invention. A person of ordinary skill in the art, would have no difficulty determining the appropriate timing, sequence and dosages of administration for particular drugs and compounds of the present invention.

As used herein “pharmaceutically acceptable carrier” includes any and all solvents, dispersion media, coatings, antibacterial and antifungal agents, isotonic and absorption delaying agents, and the like that are physiologically compatible. In one aspect, the carrier is suitable for parenteral administration. Alternatively, the carrier can be suitable for intravenous, intraperitoneal or intramuscular administration. In another aspect, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is compatible with the active compound, use thereof in the pharmaceutical compositions is contemplated. Supplementary active compounds can also be incorporated into the compositions.

A “pharmaceutically acceptable salt” refers to a salt that retains the desired biological activity of the parent compound and does not impart any undesired toxicological effects (See, e.g., Berge, et al., J. Pharm. Sci., 66: 1-19, 1977). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In prophylactic applications, pharmaceutical compositions or medicaments are administered to a patient susceptible to, or otherwise at risk of a disease or condition (i.e., as a result of bacteria, fungi, viruses, parasites or the like) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease or condition in an amount sufficient to cure, or at least partially arrest, the symptoms of the disease or condition (e.g., biochemical and/or histologic), including its complications and intermediate pathological phenotypes in development of the disease or condition. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if the response starts to wane.

The pharmaceutical composition of the present invention should be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound can be orally administered, for example, with an inert diluent or an assimilable edible carrier.

Pharmaceutical compositions of the invention also can be administered in combination therapy, i.e., combined with other agents. For example, in treatment of bacteria, the combination therapy can include a composition of the present invention with at least one agent or other conventional therapy.

### J. Routes of Administration

A composition of the present invention can be administered by a variety of methods known in the art. The route and/or mode of administration vary depending upon the desired results. The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. The peptide of the invention can be administered parenterally by injection or by gradual infusion over time. The peptide can also be prepared with carriers that protect the compound against rapid release, such as a controlled release formulation, including implants, transdermal patches, and microencapsulated delivery systems Further methods for delivery of the peptide include orally, by encapsulation in microspheres or proteinoids, by aerosol delivery to the lungs, or transdermally by iontophoresis or transdermal electroporation. To administer a peptide of the invention by certain routes of administration, it can be necessary to coat the compound with, or co-administer the compound with, a material to prevent its inactivation. The method of the invention also includes delivery systems such as microencapsulation of peptides into liposomes or a diluent. Microencapsulation also allows co-entrapment of antimicrobial molecules along with the antigens, so that these molecules, such as antibiotics, can be delivered to a site in need of such treatment in conjunction with the peptides of the invention. Liposomes in the blood stream are generally taken up by the liver and spleen. Pharmaceutically acceptable diluents include saline and aqueous buffer solutions. Liposomes include water-in-oil-in-water CGF emulsions as well as conventional liposomes (Strejan, et al., J. Neuroimmunol., 7: 27, 1984). Thus, the method of the invention is particularly useful for delivering antimicrobial peptides to such organs. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, Ed., 1978, Marcel Dekker, Inc., New York. Other methods of administration will be known to those skilled in the art.

Preparations for parenteral administration of a peptide of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred aspect, a therapeutic composition of the invention can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

When the peptides of the present invention are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

### K. Effective Dosages

“Therapeutically effective amount” as used herein for treatment of antimicrobial related diseases and conditions refers to the amount of peptide used that is of sufficient quantity to decrease the numbers of bacteria, viruses, fungi, and parasites in the body of a subject. The dosage ranges for the administration of peptides are those large enough to produce the desired effect. The amount of peptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's (Remington's Pharmaceutical Science, Mack Publishing Company, Easton, Pa.); Egleton, Peptides 18: 1431-1439, 1997; Langer Science 249: 1527-1533, 1990. The dosage regimen can be adjusted by the individual physician in the event of any contraindications.

Dosage regimens of the pharmaceutical compositions of the present invention are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the compounds of the invention employed in the pharmaceutical composition at levels lower than that required to achieve the desired therapeutic effect and gradually increase the dosage until the desired effect is achieved. In general, a suitable daily dose of a compound of the invention is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of a therapeutic composition can be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

An effective dose of each of the peptides disclosed herein as potential therapeutics for use in treating microbial diseases and conditions is from about 1 μg to 500 mg/kg body weight, per single administration, which can readily be determined by one skilled in the art. As discussed above, the dosage depends upon the age, sex, health, and weight of the recipient, kind of concurrent therapy, if any, and frequency of treatment. Other effective dosage range upper limits are 100 mg/kg body weight, 50 mg/kg body weight, 25 mg/kg body weight, and 10 mg/kg body weight.

The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Some compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (See, e.g., Ranade, J. Clin. Pharmacol., 29: 685, 1989). Exemplary targeting moieties include folate or biotin (See, e.g., U.S. Pat. No. 5,416,016 to Low, et al.); mannosides (Umezawa, et al., Biochem. Biophys. Res. Commun., 153: 1038, 1988); antibodies (Bloeman, et al., FEBS Lett., 357: 140, 1995; Owais, et al., Antimicrob. Agents Chemother., 39: 180, 1995); surfactant protein A receptor (Briscoe, et al., Am. J. Physiol., 1233: 134, 1995), different species of which can comprise the formulations of the inventions, as well as components of the invented molecules; p120 (Schreier, et al., J. Biol. Chem., 269: 9090, 1994); See also Keinanen, et al., FEBS Lett., 346: 123, 1994; Killion, et al., Immunomethods, 4: 273, 1994. In some methods, the therapeutic compounds of the invention are formulated in liposomes; in a more preferred aspect, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

“Bactericidal amount” as used herein refers to an amount sufficient to achieve a bacteria-killing blood concentration in the subject receiving the treatment. The bactericidal amount of antibiotic generally recognized as safe for administration to a human is well known in the art, and as is known in the art, varies with the specific antibiotic and the type of bacterial infection being treated.

Because of the antibiotic, antimicrobial, and antiviral properties of the peptides, they can also be used as preservatives or sterillants of materials susceptible to microbial or viral contamination. The peptides of the invention can be utilized as broad spectrum antimicrobial agents directed toward various specific applications. Such applications include use of the peptides as preservatives in processed foods (organisms including Salmonella, Yersinia, Shigella), either alone or in combination with antibacterial food additives such as lysozymes; as a topical agent (Pseudomonas, Streptococcus) and to kill odor producing microbes (Micrococci). The relative effectiveness of the peptides of the invention for the applications described can be readily determined by one of skill in the art by determining the sensitivity of any organism to one of the peptides.

### L. Formulation

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al, European Journal Immunology 25: 3521-24, 1995; Cevc et al, Biochimica Biophysica Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### M. Kits

The invention provides kits comprising the compositions, e.g., nucleic acids, expression cassettes, vectors, cells, polypeptides (e.g., an isolated immunomodulatory bacteriocin or lantibiotic peptide with net cationic charge) of the invention and the like. The isolated immunomodulatory bacteriocin or lantibiotic peptide can have an amino acid sequence of SEQ ID NOS: 1-6, or be analogs, derivatives, amidated variations or conservative variations thereof. The kits also can contain instructional material teaching the methodologies and uses of the invention, as described herein.

From the foregoing description, various modifications and changes in the compositions and methods will occur to those skilled in the art. All such modifications coming within the scope of the appended claims are intended to be included therein. Each recited range includes all combinations and sub-combinations of ranges, as well as specific numerals contained therein.

All publications and patent documents cited above are hereby incorporated by reference in their entirety for all purposes to the same extent as if each were so individually denoted.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

### EXEMPLARY ASPECTS

### Example 1

### Enhancement of Innate Immunity

The natural human peptide LL-37 (Bowdish, D M E, D J Davidson, Y E Lau, K Lee, MG Scott, and REW Hancock. 2005, J. Leukocyte Biol. 77:451-459). as well as the synthetic peptide IDR-1 (Scott, M G, et al. 2007, Nature Biotech. 25: 465-472.) are able to protect against bacterial infections despite having no antimicrobial activity under physiological conditions. It appears to manifest this activity due to their ability to induce the production of certain chemokines which are able to recruit subsets of cells of innate immunity to infected tissues. Therefore we tested if the novel peptides described here had the ability to induce chemokine production in human peripheral blood mononuclear cells.

Venous blood (20 ml) from healthy volunteers was collected in Vacutainer® collection tubes containing sodium heparin as an anticoagulant (Becton Dickinson, Mississauga, ON) in accordance with UBC ethical approval and guidelines. Blood was diluted 1:1 with complete RPMI 1640 medium and separated by centrifugation over a Ficoll-Paque® Plus (Amersham Biosciences, Piscataway, N.J., USA) density gradient. White blood cells were isolated from the buffy coat, washed twice in RPMI 1640 complete medium, and the number of peripheral blood mononuclear cells (PBMC) was determined by trypan blue exclusion. PBMC (5×10⁵) were seeded into 12-well tissue culture dishes (Falcon; Becton Dickinson) at 0.75 to 1×10⁶ cells/ml at 37° C. in 5% CO₂. The above conditions were chosen to mimic conditions for circulating blood monocytes entering tissues at the site of infection via extravasation.

Following incubation of the cells under various treatment regimens, the tissue culture supernatants were centrifuged at 1000×g for 5 min, then at 10,000×g for 2 min to obtain cell-free samples. Supernatants were aliquoted and then stored at −20° C. prior to assay for various chemokines by capture ELISA (eBioscience and BioSource International Inc., CA, USA respectively).

Peptides (Table 1) were purified from 3 L bacterial fermentation broths. For Pep5, the producer strain S. epidermidis 25 was grown in tryptic soy broth (TSB, Merck, Darmstadt, Germany) at 36° C. with aeration. The peptide was purified by subjecting the culture supernatant to hydrophobic interaction (XAD 1) and CM Sephadex cation exchange chromatography as described (Sahl H-G and H Brandis. 1981. Production, purification, and chemical properties of an antistaphylococcal agent produced by Staphylococcus epidermidis. Journal of General Microbiology, 127, 377-383). Subsequent purification on reversed phase HPLC was performed as described (Sahl, H-G, M Groβgarten, W R Widger, W A Cramer, H Brandis. 1985. Structural similarities of the staphylococcin-like peptide Pep 5 to the peptide antibiotic Nisin. Antimicrobial Agents and Chemotherapy 27, 836-840). Gallidermin was purified from Staphylococcus gallinarum Tü 3928 cultivated in TSB and nisinZ from L. lactis NIZO 22186 cultivated in SPYS medium (3% sucrose 1% peptone, 1% yeast extract, 1% potassium phosphate buffer, adjusted to pH 6.8). Cells were harvested by centrifugation (10,000×g, 10 min) and the peptides were extracted from the culture supernatant. Chloroform was added to the supernatant fluid (0.1:1 v/v), stirred vigorously for 1 h at 4° C., and centrifuged (10,000×g, 10 min) for phase separation. The precipitate formed at the interface between the chloroform and culture supernatant fluid was lyophilised. The crude extract was resuspended in 30% acetonitrile 0.1% trifluoroacetic acid (TFA) and applied to a preparative high-performance liquid chromatography column (Nucleosil 100-C18-10 μm 225×20 mm ID; Schambeck S F D, Bad Honnef, Germany). The column was equilibrated with buffer A (H₂O, 0.1% [vol/vol] TFA) and peptides were eluted using a linear gradient of 20-60% buffer B (acetonitrile, 0.1% [vol/vol] TFA) at a flow rate of 12 ml/min. For further purification a semi-preparative (Nucleosil 100-5C18 250×8.6 mm ID) and a analytical (Nucleosil 100-3C18 250×4.6 mm ID) column was used. MALDI TOF mass spectrometry was used to confirm the correct mass and the purity of the peptides. Stock solutions were prepared in 0.05% acetic acid and stored at −20°

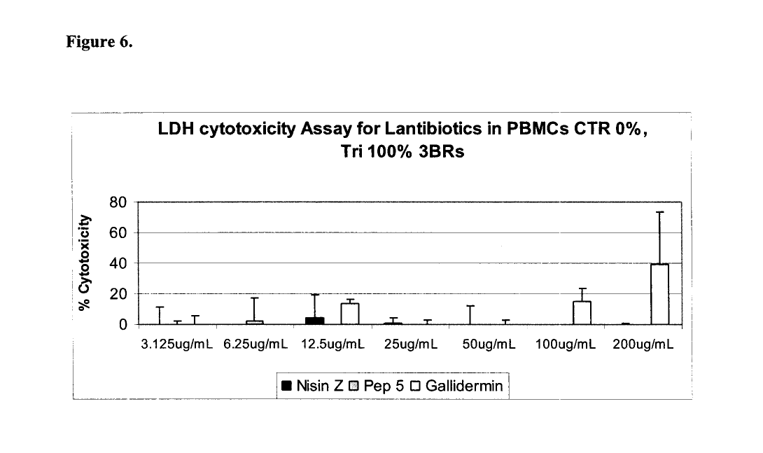

As shown in FIG. 2A-C, all peptides (SEQ ID NO: 1-3), compared to the human host defence peptide LL-37 showed equal or far superior abilities to stimulate human PBMC to induce the expression (as assessed by ELISA 24 hours after peptide addition) of the chemokines MCP-1, Gro-α and IL8. Nisin Z was the most effective even at the lowest peptide concentration utilized (5 μg/ml) and showed a good dose response between 30 and 150 μg/ml, while gallidermin was also quite effective. Similarly SEQ ID NOS: 4-6 (FIG. 7) all demonstrated an ability to induce MCP-1, with the former 3 and especially Nisin A, a variant of Nisin Z demonstrating excellent activity. None of the peptides demonstrated substantial toxicity against human PBMC (FIG. 6).

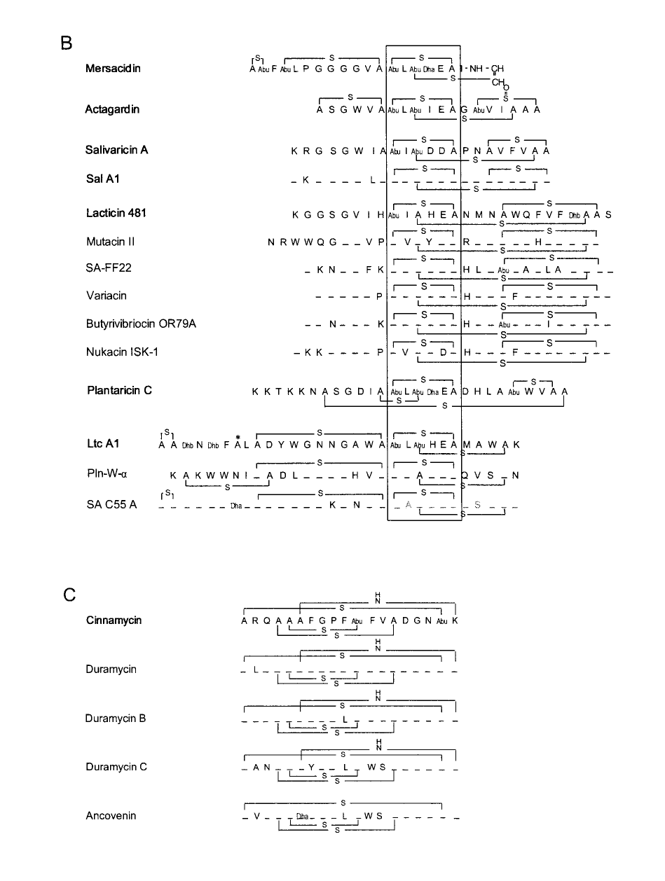

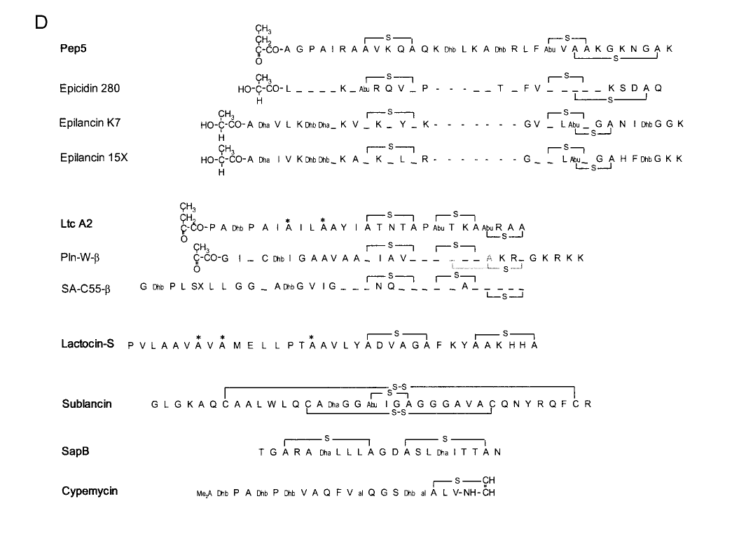

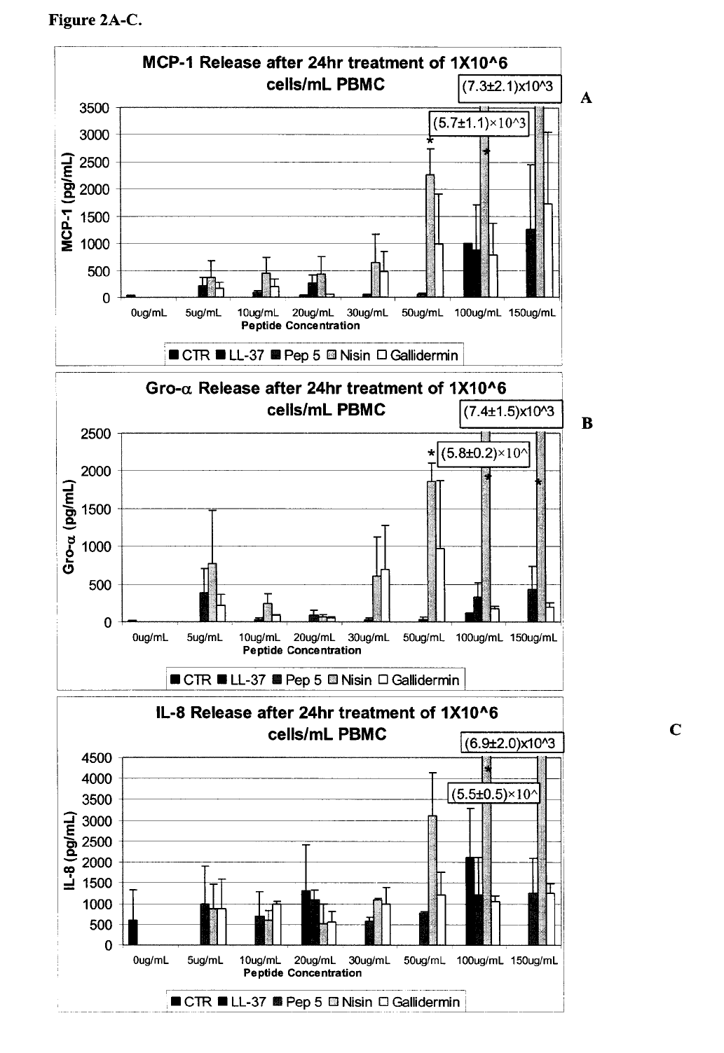

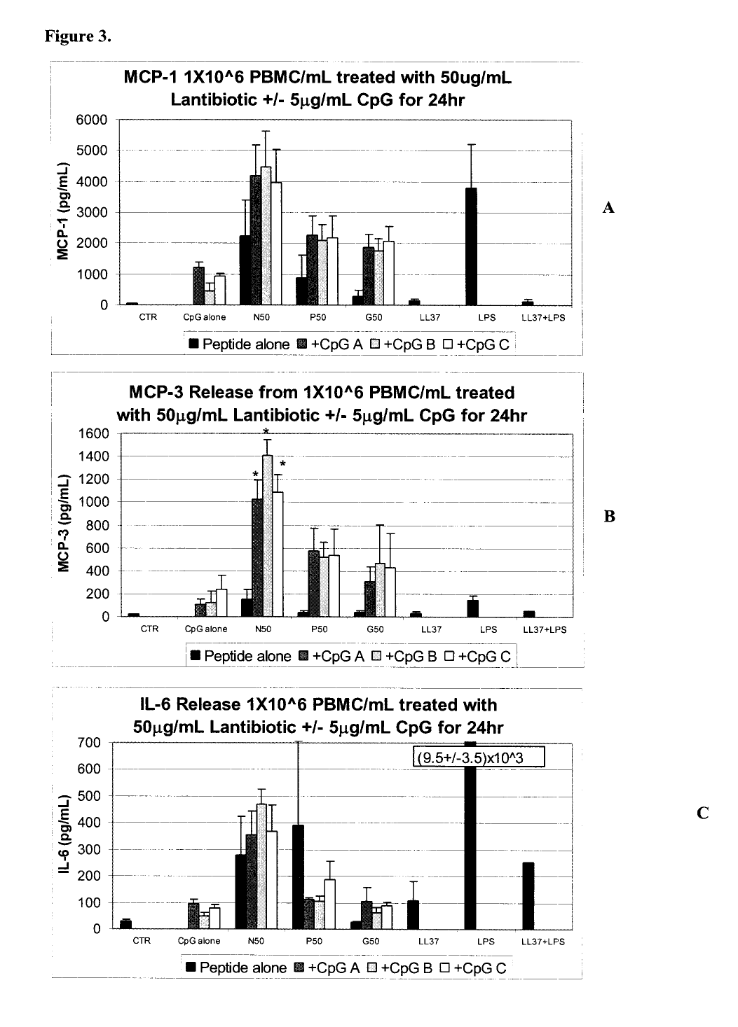

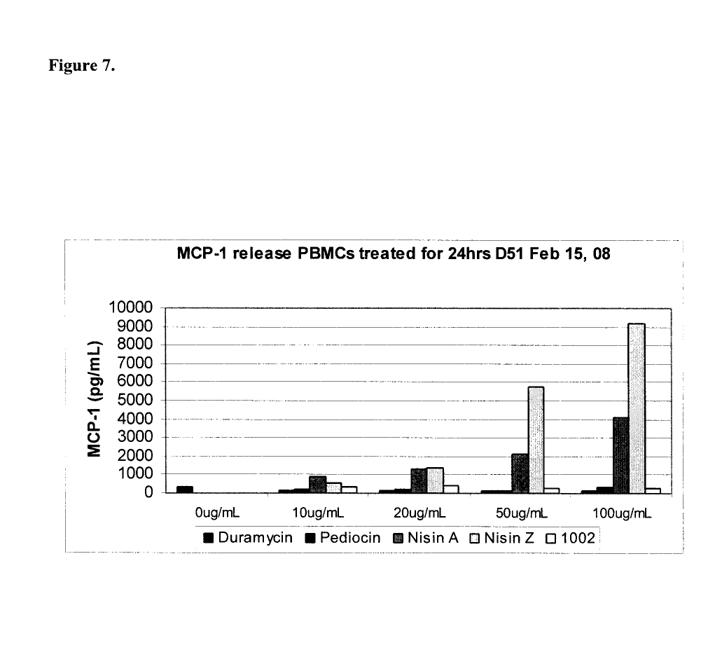

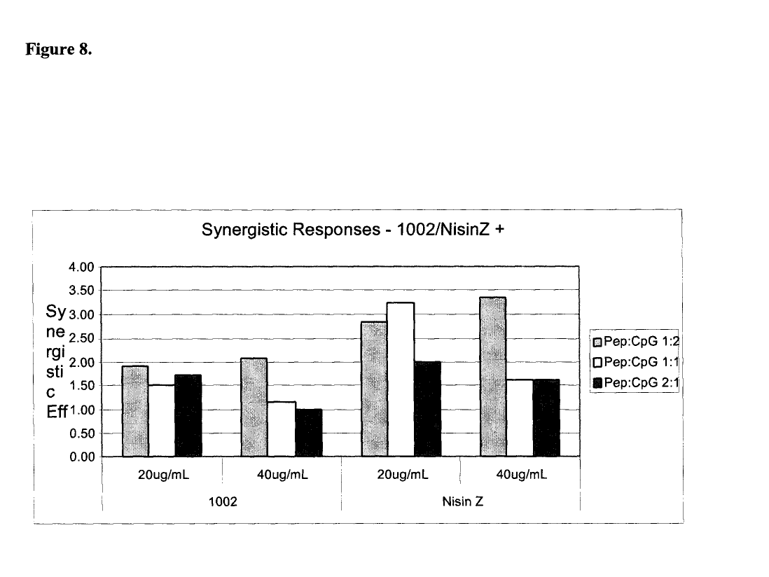

Stimulation of MCP-1 has been shown to have some relationship to activity in vivo as an adjuvant as well as ability to directly protect against infections. As shown in FIG. 3A-C, all lantibiotics worked additively with the three classes of CpG oligonucleotides tested, increasing the ability of these oligonucleotide innate immune modulators, which are known to bind to Toll like receptor TLR9, to mildly stimulate innate immunity. It is worth noting that Nisin Z was superior to any of the three classes of CpG in inducing MCP-1 and IL6. Similarly Nisin Z statistically significantly (p<0.05) increased MCP-3 release above the additive effect of CpG alone and lantibiotic alone. Many different adjuvant formulations were tested and it was found that rations of 2:1, 1:1 and 1:2 nisin to CpG all demonstrated synergy in inducing MCP-1 over and above the individual components (FIG. 8), with the latter formulation being sufficient.

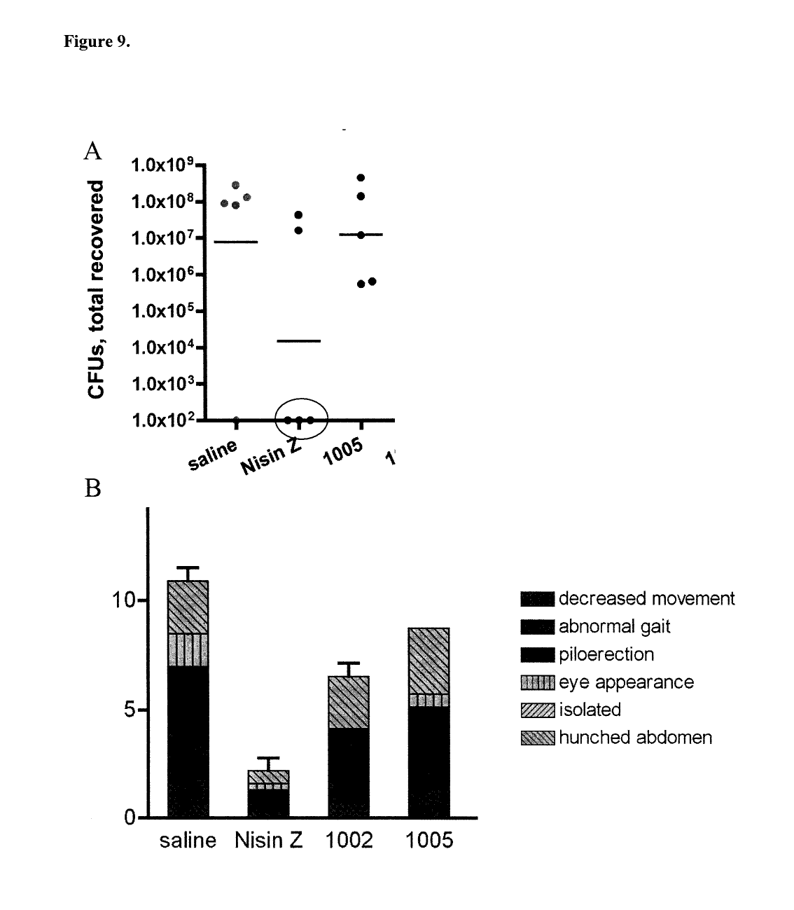

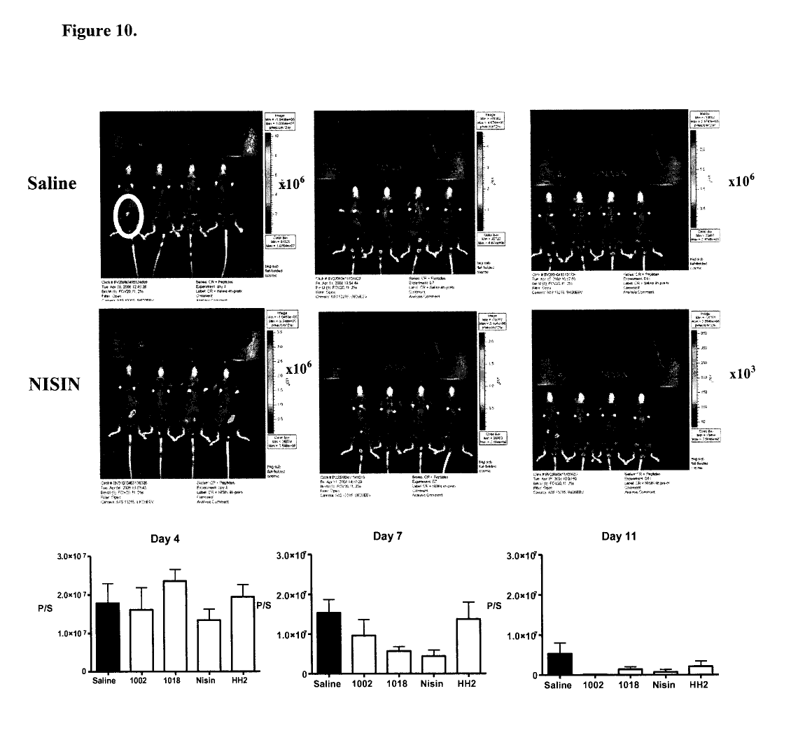

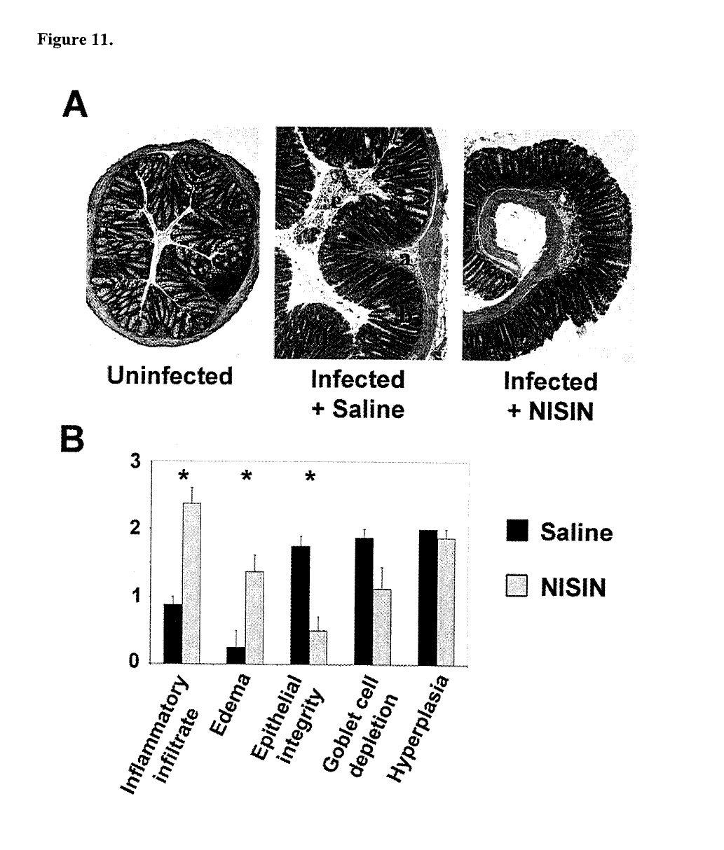

Conventional peptides are known to protect against infection (Scott et al. Nature Biotech 2007). However such peptides are very expensive whereas lantibiotics are bacterial fermentation products and thus inexpensive. Thus we assessed the reduction in colony counts after 24 hours within the peritoneum of mice treated with the lantibiotic peptide nisin (or negative control 1005 or positive control 1002) and challenged these mice 4 hours later with ˜10⁸ S. aureus in hog gastric mucin. The results show (FIG. 9) that nisin protected mice against S. aureus by reducing average colony counts. Furthermore an examination of physiological consequences (visual observation scores) indicated that these were vastly improved by treatment with nisin (cf both of the control peptides). Thus the in vitro activities of nisin in increasing MCP-1 production in PBMC is clearly related to in vivo protection in animal models. This was further confirmed by protection in a Citrobacter animal model where the peptide was introduced into the peritoneum and led to a substantial decrease in infection in the gut that lasted for up to 11 days (FIG. 10). Thus these lantibiotic peptides are clearly highly effective in stimulating innate immunity to protect vs infection, a property unrelated to any antimicrobial activity they can possess as lantibiotics are well known and were confirmed here to have no direct antimicrobial activity against Gram negative bacteria like Citrobacter.

### Example 2

### Anti-Septic Impact on Innate Immunity

It is well known that cationic antimicrobial peptides have the ability to boost immunity while suppressing septic responses to bacterial pathogen associated molecular pattern molecules like lipopolysaccharide and lipoteichoic acids as well as reducing inflammation and endotoxaemia (Finlay, B. B., and R. E. W. Hancock. 2004, Nature Microbiol. Rev. 2:497-504).

Small 12-mer peptides like Bac2A and 13-mer peptides like indolicidin have been previously shown in our laboratory to have rather modest anti-endotoxic activity, which can be assessed by measuring the ability of the peptide to suppress the LPS-stimulated production of TNFα by macrophages. It is well known for other cationic antimicrobial peptides that this corresponds to anti-endotoxic activity in reversing lethal endotoxaemia in animal models (Gough M, Hancock R E W, and N M Kelly. 1996, Infect. Immun. 64, 4922-4927). In contrast LL-37 is known to have excellent anti-endotoxic activity in vitro, as assessed by its ability to suppress the LPS-mediated induction of TNFα in monocytic cells and this is reflected by its ability to both reduce endotoxin mediated TNFα induction and lethality in a mouse model (Scott M G, D J Davidson, M R Gold, D Bowdish, and REW Hancock. 2002, Journal of Immunology 169:3883-3891). A selection of peptides were tested and some of these indeed had excellent anti-endotoxic activity (FIG. 3).

LPS from P. aeruginosa strain H103 was highly purified free of proteins and lipids using the Darveau-Hancock method. Briefly, P. aeruginosa was grown overnight in LB broth at 37° C. Cells were collected and washed and the isolated LPS pellets were extracted with a 2:1 chloroform:methanol solution to remove contaminating lipids. Purified LPS samples were quantitated using an assay for the specific sugar 2-keto-3-deoxyoctosonic acid (KDO assay) and then resuspended in endotoxin-free water (Sigma-Aldrich).

PBMC were stimulated with LPS (2 or 100 ng/ml) with or without peptide (50 μg/ml) for 4 or 24 hours as indicated below. Following incubation of the cells under various treatment regimens, the tissue culture supernatants were centrifuged at 1000×g for 5 min, then at 10,000×g for 2 min to obtain cell-free samples. Supernatants were aliquoted and then stored at −20° C. prior to assay for various cytokines. TNFα secretion was detected with a capture ELISA (eBioscience and BioSource International Inc., CA, USA respectively).

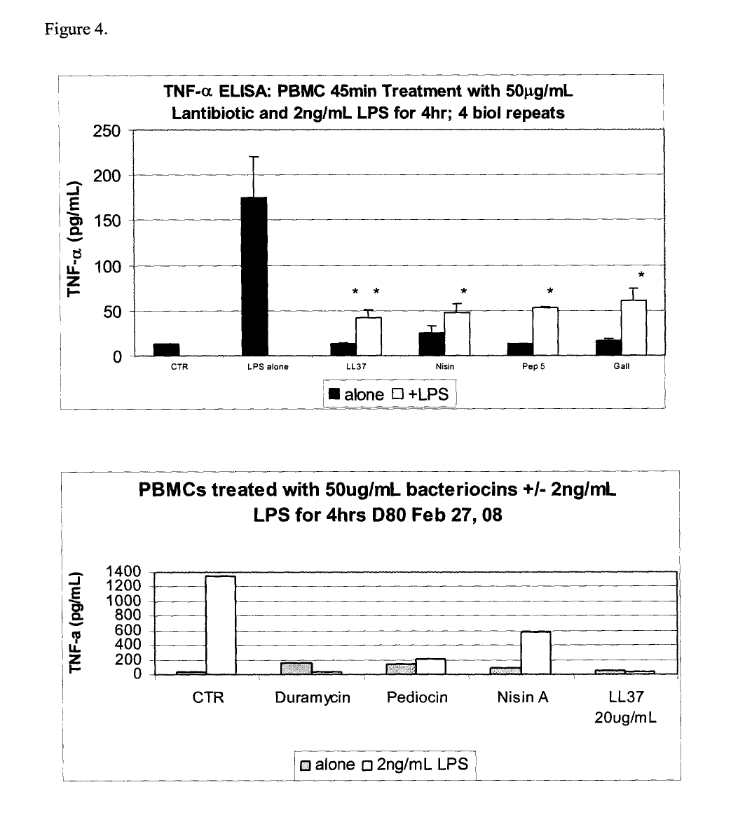

As shown in FIG. 4, unlike the bacterial endotoxin LPS, none of the six peptides induced substantial levels of TNFα, a classical pro-inflammatory cytokine which has been associated with sepsis. In contrast, like the potent anti-endotoxin peptide LL-37, they actually suppressed LPS induced production of TNFα by 50-90% when added at the same time as LPS and assayed by ELISA 4 hours later.

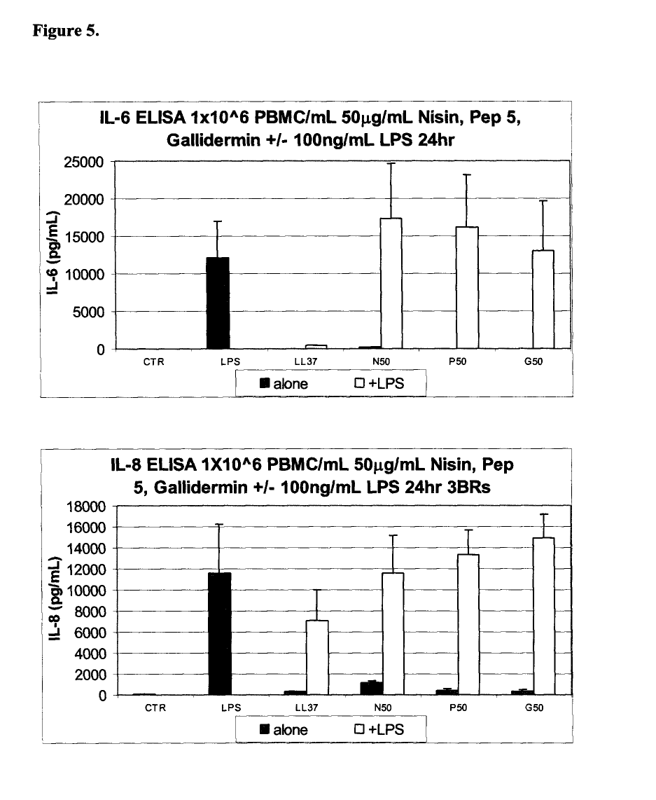

As shown in FIG. 5, the peptides when assayed 24 hours after treatment of PBMC did not induce levels of IL6 or IL8 to the extent observed with bacterial endotoxic LPS present at 100 ng/ml, which is the usual concentration used by immunologists to stimulate innate immunity. Moreover there was no substantial enhancement of responsiveness to LPS when peptides were added simultaneously.

None of the peptides showed any evidence of cytotoxicity toward PBMC as assessed by LDH release (FIG. 6) and visual inspection of cells.

### Example 3

### Adjuvant Activities

Adjuvants are critical components of both whole killed vaccines and subunit vaccines. Adjuvants can be categorized into delivery vehicles and immunomodulators according to their chemical nature. Vehicles including liposomes, emulsions, and ISCOMS, help to carry and retain antigens in close proximity to the lymphoid tissues (depot). Immune modulators such as CpG ODN, muramyldipeptide (MDP) and monophosphoryl lipid A (MPL) stimulate local secretion of cytokines and condition the vaccination site. Adjuvants stimulate either the innate or specific immune response through different mechanisms.

Stimulation of innate immunity usually occurs through signaling via Pathogen recognition receptors (PRRs) that recognize conserved pathogen signature molecules such as LPS or lipoteichoic acid. PRRs include proteins that are associated with complement and opsonization, surface receptors on phagocytic cells that are associated with endocytosis, or Toll like receptors (TLR). Signaling through these receptors leads to activation of the nuclear factor-κB (NF-κB) which results in the expression of various cytokines, chemokines and co-stimulatory molecules. This response limits spread of the invading infectious agent until the adaptive immune response is developed. However, recognition of PAMPs often requires an adaptor protein such as LPS binding protein or CD14. Moreover, recognition can also occur by more than one TLR resulting in cooperation of different TLRs. Thus, binding of adjuvant PAMPs by TLRs stimulates innate immunity, which, in turn, activates adaptive immunity. In addition adaptive immunity can be directly stimulated by certain vehicle-type adjuvants, such as amphipathic non-ionic polymers or saponin, which bind to exogenous antigens and therefore preserve their 3-dimensional conformation during internalization by antigen-presenting cells (APCs).

A number of adjuvants that are currently used experimentally for mucosal delivery, including cholera toxin A subunit, E. coli heat labile toxin or MF59, are reasonably effective, but can find limited applications due to safety concerns. Cationic host defence peptides including defensins have been demonstrated to have a plethora of immunomodulatory activities in innate immunity, including an ability to stimulate chemotaxis of immature DCs and T-cells, glucocorticoid production, macrophage phagocytosis, mast cell degranulation, complement activation and IL-8 production by epithelial cells, and to moderate antimicrobial activities that are particularly important at the high concentrations present within phagocytic granules or the crypts of the intestine. It has also been reported that one defensin chemoattracted monocytes, DCs and T cells by acting through the chemokine receptor (CCR) 6 (Yang D et al. 1999, Science 286:525-8.), while other host defense peptides mediate chemotaxis directly through other or unknown receptors or through chemokine induction in host cells (Bowdish, D M E, D J Davidson, and REW Hancock. 2006, Current Topics in Microbiology and Immunology 306:27-66). Thus, defensins appear to represent an important link between innate and acquired immunity and are potent immune modulators and adjuvants for vaccines. Consistent with this, low concentrations of human α-defensins (10-1000 ng), administered with KLH absorbed to alum, lead to strong augmentation of IgG1, IgG2a and IgG2b, indicative of stimulation of both Th1 and Th2 responses (Tani K et al. 2000, International Immunology 12:691-700.). Unfortunately, defensins contain three disulphide bonds and thus are relatively expensive to manufacture.

In contrast, we have demonstrated here that certain lantibiotic bacteriocins have immunomodulatory activities reminiscent of those found in defensins, and are inexpensive to manufacture and therefore excellent adjuvant candidates. Therefore it can be concluded that Nisin Z, Pep5, and gallidermin will have adjuvant activity due to their ability to induce chemokines (FIG. 2) and furthermore show synergy with known adjuvants like CpG (FIG. 3, FIG. 8).

TABLE 2
Sequences of cationic bacteriocins

>gi|38352189|gb|AAR18691.1| bacteriocin (Serratia marcescens)
MSGGDGRGPGNSGLGHNGGQAR
K:0 R:2 D:1 E:0
##########################################################################
>gi|972711|gb|AAB81304.1| bacteriocin (Carnobacterium piscicola)
MKIKTITKKQLIQIKGGSKNSQIGKSTSSISKCVFSFFKKC
K:10 R:0 D:0 E:0
##########################################################################
>gi|972709|gb|AAB81302.1| bacteriocin (Carnobacterium piscicola)
MNKEFKSLNEVEMKKINGGSAILAITLGIFATGYGMGVQKAINDRRKK
K:7 R:2 D:1 E:3
##########################################################################
>gi|122004997|gb|ABM65805.1| bacteriocin (Salmonella enteritidis)
KRGRAPYSLIRQQVGGRWTYEIPHVGKIQYGGMVFDVDNLMINTPK
K:3 R:4 D:2 E:1
##########################################################################
>gi|122004995|gb|ABM65804.1| bacteriocin (Salmonella enterica subsp.
enterica serovar Washington)
KRGRAPYSLIRQQVGGRWTYEIPHVGKIQYGGMVFDVDNLMINTPK
K:3 R:4 D:2 E:1
##########################################################################
>gi|122004993|gb|ABM65803.1| bacteriocin (Salmonella typhimurium)
KRGRAPYSLIRQQVGGRWTYEIPHVGKIQYGGMVFDVDNLMINTPK
K:3 R:4 D:2 E:1
##########################################################################
>gi|122004991|gb|ABM65802.1| bacteriocin (Salmonella paratyphi)
KRGRAPYSLIRQQVGGRWTYEIPHVGKIQYGGMVFDVDNLMINTPK
K:3 R:4 D:2 E:1
##########################################################################
>gi|122004989|gb|ABM65801.1| bacteriocin (Salmonella typhi)
KRGRAPYSLIRQQVGGRWTYEIPHVGKIQYGGMVFDVDNLMINTPK
K:3 R:4 D:2 E:1
##########################################################################
>gi|122004987|gb|ABM65800.1| bacteriocin (Salmonella typhi)
KRGRAPYSLIRQQVGGRWTYEIPHVGKIQYGGMVFDVDNLMINTPK
K:3 R:4 D:2 E:1
##########################################################################
>gi|1172531|sp|P80214|PLNA_LACPL Bacteriocin plantaricin-A precursor
MKIQIKGMKQLSNKEMQKIVGGKSSAYSLQMGATAIKQVKKLFKKWGW
K:11 R:0 D:0 E:1
##########################################################################
>gi|20532149|sp|P83002|LCNM_LACLA Bacteriocin lactococcin MMFII
TSYGNGVHCNKSKCWIDVSELETYKAGTVSNPKDILW
K:4 R:0 D:2 E:2
##########################################################################
>gi|3123187|sp|P80493|BAVM_LACSK Bacteriocin bavaricin-MN
TKYYGNGVYCNSKKCWVDWGQAAGGIGQTVVXGWLGGAIPGK
K:4 R:0 D:1 E:0
##########################################################################
>gi|27808660|sp|P81053|LCCC_LEUME Bacteriocin leucocin C
KNYGNGVHCTKKGCSVDWGYAWTNIANNSVMNGLTGGNAGWHN
K:3 R:0 D:1 E:0
##########################################################################
>gi|115502148|sp|P84962|DIV35_CARDV Bacteriocin divergicin M35
TKYYGNGVYCNSKKCWVDWGTAQGCIDVVIGQLGGGIPGKGKC
K:5 R:0 D:2 E:0
##########################################################################
>gi|3122418|sp|P80925|MUTI_ENTMU Bacteriocin mundticin
KYYGNGVSCNKKGCSVDWGKAIGIIGNNSAANLATGGAAGWSK
K:5 R:0 D:1 E:0
##########################################################################
>gi|2493155|sp|P80953|BAVA_LACSK Bacteriocin bavaricin-A
KYYGNGVHXGKHSXTVDWGTAIGNIGNNAAANXATGXNAGG
K:2 R:0 D:1 E:0
##########################################################################
>gi|110808192|sp|P84886|CURVA_LACCU Bacteriocin curvaticin
AYPGNGVHCGKYSCTVDKQTAIGNIGNNAA
K:2 R:0 D:1 E:0
##########################################################################
>gi|2493157|sp|P80959|LC70_LACPA Bacteriocin lactocin-705
GMSGYIQGIPDFLKGYLHGISAANKHKKGRL
K:4 R: D:1 E:0
##########################################################################
>gi|547832|sp|P36962|LCGB_LACLA Bacteriocin lactococcin-G subunit beta
KKWGWLAWVDPAYEFIKGFGKGAIKEGNKDKWKNI
##########################################################################
K:8 R:0 D:2 E:2
>gi|547831|sp|P36961|LCGA_LACLA Bacteriocin lactococcin-G subunit alpha
GTWDDIGQGIGRVAYWVGKAMGNMSDVNQASRINRKKKH
K:4 R:3 D:3 E:0
##########################################################################
>gi|21759225|sp|Q48501|LA89_LACAC Bacteriocin acidocin 8912 precursor
MISSHQKTLTDKELALISGGKTHYPTNAWKSLWKGFWESLRYTDGF
K:5 R:1 D:2 E:2
##########################################################################
>gi|48428801|sp|P83375|BSP43_SERPL Bacteriocin serracin-P 43 kDa subunit
DYHHGVRVL
K:0 R:1 D:1 E:0
##########################################################################
>gi|48428802|sp|P83378|BSP23_SERPL Bacteriocin serracin-P 23 kDa subunit
ALPKKLKYLNLFNDGFNYMGVV
K:3 R:0 D:1 E:0
##########################################################################
>gi|585018|sp|P80323|CU47_LACCU Bacteriocin curvaticin FS47
YTAKQCLQAIGSCGIAGTGAGAAGGPAGAFVGAXVVXI
K:1 R:0 D:0 E:0
##########################################################################
>gi|3122337|sp|P81052|LCCB_LEUME Bacteriocin leucocin-B
KGKGFWSWASKATSWLTGPQQPGSPLLKKHR
K:5 R:1 D:0 E:0
##########################################################################
>gi|l094028|prf||2105253A bacteriocin
KYYGNGVTCGKHSCSVDXGKATTCIINNGAMAXATGGHQGNHKC
K:4 R:0 D:1 E:0
##########################################################################
>gi|861526|gb|AAB32666.1| bacteriocin lactacin B inducer {N-terminal}
(Lactobacillus acidophilus, N2, Peptide Partial, 19 aa)
SRTPIIAGNWKLNMNPKET
K:2 R:1 D:0 E:1
##########################################################################
>gi|78609814|emb|CAI54861.1| Putative bacteriocin inducing peptide
(Lactobacillus sakei subsp. sakei 23K)
MMIFKKLSEKELQKISGGVGIQKCSLGFSSREYLNKITKWIKHH
K:8 R:1 D:0 E:3
##########################################################################
>gi|81428172|ref|YP_395172.1| Putative bacteriocin inducing peptide
(Lactobacillus sakei subsp. sakei 23K)
MMIFKKLSEKELQKISGGVGIQKCSLGFSSREYLNKITKWIKHH
K:8 R:1 D:0 E:3
##########################################################################
>gi|1703706|gb|AAB37715.1| enterocin CRL 35 = pediocin-like bacteriocin
{N-terminal} (Enterococcus faecium, CRL 35, Argentinian Tafi cheese
isolate, Peptide Partial, 21 aa)
KYYGNGVTLNKXGXSVNXXXA
K:2 R:0 D:0 E:0
##########################################################################
>gi|264292|gb|AAB25127.1| mesentericin Y105 = anti-Listeria bacteriocin
(Leuconostoc mesenteroides, ssp. mesenteroides, Y105, Peptide, 36 aa)
KYYGNGVHCTKSGCSVNWGEAASAGIHRLANGGNGF
K:2 R:1 D:0 E:1
##########################################################################
>gi|547826|sp|P36960|LANC_CARUI Lantibiotic carnocin UI49
GSEIQPR
K:0 R:1 D:0 E:1
##########################################################################
>gi|81174729|sp|P0C0H9|SRTA_STRP1 Lantibiotic streptin precursor
MNNTIKDFDLDLKTNKKDTATPYVGSRYLCTPGSCWKLVCFTTTVK
K:6 R:1 D:4 E:0
##########################################################################
>gi|1174653|sp|P42723|TFXA_RHILT Trifolitoxin precursor (TFX)
MDNKVAKNVEVKKGSIKATFKAAVLKSKTKVDIGGSRQGCVA
K:9 R:1 D:2 E:1
##########################################################################
>gi|76364234|sp|P0C0H8|SRTA_STRPY Lantibiotic streptin precursor
MNNTIKDFDLDLKTNKKDTATPYVGSRYLCTPGSCWKLVCFTTTVK
K:6 R:1 D:4 E:0
##########################################################################
>gi|2497613|sp|P80666|LANM_STRMU Lantibiotic mutacin B-Ny266
FKSWSFCTPGCAKTGSFNSYCC
K:2 R:0 D:0 E:0
##########################################################################
>gi|729916|sp|P38655|LANC_STRS6 Lantibiotic ancovenin
CVQSCSFGPLTWSCDGNTK
K:1 R:0 D:1 E:0
##########################################################################
>gi|544195|sp|P36503|DURC_STRGP Lantibiotic duramycin-C
CANSCSYGPLTWSCDGNTK
K:1 R:0 D:1 E:0
##########################################################################
>gi|544194|sp|P36502|DURB_STRGW Lantibiotic duramycin-B
CRQSCSFGPLTFVCDGNTK
K:1 R:1 D:1 E:0
##########################################################################
>gi|544193|sp|P36504|DURA_STRGV Lantibiotic duramycin (Leucopeptin)
(Antibiotic PA48009)
CKQSCSFGPFTFVCDGNTK
K:2 R:0 D:1 E:0
##########################################################################
>gi|7476021|pir||S77569 plantaricin SA6 - Lactobacillus plantarum
(strain SA6) (fragment)
VYPFPGPIXMANLVLTXLSHLHRSTVXFS
K:0 R:1 D:0 E:0
##########################################################################
>gi|49245942|gb|AAT58220.1| enterocin P-like protein
(Enterococcus faecium)
ATRSYDNGIYCNNSKCWVNWGEAKENIAGIVISGWASGLAGMGH
K:2 R:1 D:1 E:2
##########################################################################
61
>gi|2781283|pdb|3LEU| High Resolution lh Nmr Study Of Leucocin A In
Dodecylphosphocholine Micelles, 19 Structures (1:40 Ratio Of Leucocin
A:dpc) (0.1% Tfa)
KYYGNGVHCTKSGCSVNWGEAFSAGVHRLANGGNGFW
K:2 R:1 D:0 E:1
##########################################################################
>gi|2781282|pdb|2LEU| High Resolution lh Nmr Study Of Leucocin A In
90% Aqueous Trifluoroethanol (Tfe) (0.1% Tfa), 18 Structures
KYYGNGVHCTKSGCSVNWGEAFSAGVHRLANGGNGFW
K:2 R:1 D:0 E:1
##########################################################################
>gi|1495681|emb|CAA64194.1|PlnV (Lactobacillus plantarum)
MVHQNVKFISRLLLASLLAAIVMGLSTAPIDILTLKYNWITVAI
K:2 R:1 D:1 E:0
##########################################################################
>gi|1495669|emb|CAA64204.1| plantaricin A precursor peptide
(Lactobacillus plantarum)
MKIQIKGMKQLSNKEMQKIVGGKSSAYSLQMGATAIKQVKKLFKKWGW
K:11 R:0 D:0 E:1
##########################################################################
>gi|1495660|emb|CAA64195.1| PlnR (Lactobacillus plantarum)
MLNKTINI1KKYPVRSLLVALIVVFAIYVISDPSIISSFNQGLSDGAAGR
K:3 R:2 D:2 E:0
##########################################################################
>gi|258566|gb|AAB23877.1| pediocin PA-1 = bacteriocin (Pediococcus
acidilactici, Peptide, 44 aa)
KYYGNGVTCGKHSCSVDWGKATTCIINNGAMAXATGGHQGNHKX
K:4 R:0 D:1 E:0
##########################################################################
>gi|42741977|gb|AAS45210.1| mature divercin RV41 (synthetic construct)
MDPTKYYGNGVYCNSKKCWVDWGQASGCIGQTVVGGWLGGAIPGKC
K:4 R:0 D:2 E:0
##########################################################################
>gi|19911781|dbj|BAB88211.1| mundticin KS precursor (Enterococcus mundtii)
MSQVVGGKYYGNGVSCNKKGCSVDWGKAIGIIGNNSAANLATGGAAGWKS
K:5 R:0 D:1 E:0
##########################################################################
>gi|32453480|ref|NP_861549.1| LsbB (Lactococcus lactis subsp. lactis)
MKTILRFVAGYDIASHKKKTGGYPWERGKA
K:5 R:2 D:1 E:1
##########################################################################
>gi|27544861|gb|AAO18427.1| plantaricin NC8 alpha peptide precursor
(Lactobacillus plantarum)
MDKFEKISTSNLEKISGGDLTTKLWSSWGYYLGKKARWNLKHPYVQF
K:7 R:1 D:2 E:2
##########################################################################
>gi|31994091|gb|AAP73814.1| LsbB (Lactococcus lactis subsp. lactis)
MKTILRFVAGYDIASHKKKTGGYPWERGKA
K:5 R:2 D:1 E:1
##########################################################################
>gi|599567|dbj|BAA07737.1| acidocin8912 (Lactobacillus acidophilus)
MISSHQKTLTDKELALISGGKTHYPTNAWKSLWKGFWESLRYTDGF
K:5 R:1 D:2 E:2
##########################################################################
>gi|145411501|gb|ABP68408.1| enterocin J (Enterococcus faecalis)
MGAIAKLVAKFGWPFIKKFYKQIMQFIGQGWTIDQIEKWLKRH
K:7 R:1 D:1 E:1
##########################################################################
>gi|78522998|gb|ABB46251.1|enterocin J (Enterococcus faecium)
MGAIAKLVTKFGWPLIKKFYKQIMQFIGQGWTIDQIEKWLKRH
K:7 R:1 D:1 E:1
##########################################################################
>gi|78522997|gb|ABB46250.1| enterocin I (Enterococcus faecium)
MGAIAKLVAKFGWPIVKKYYKQIMQFIGEGWAINKIIEWIKKHI
K:8 R:0 D:0 E:2
##########################################################################
>gi|118738559|gb|ABL11218.1| enterocin 62-6B (Enterococcus faecium)
MGAIAKLVTKFGWPLIKKFYKQIMQFIGQGWTIDQIEKWLKRH
K:7 R:1 D:1 E:1
##########################################################################
>gi|118738558|gb|ABL11217.1| enterocin 62-6A (Enterococcus faecium)
MGAIAKLVAKFGWPIVKKYYKQIMQFIGEGWAINKIIEWIKKHI
K:8 R:0 D:0 E:2
##########################################################################
>gi|32454940|gb|AAP83165.1| EntQ (Enterococcus faecium)
MNFLKNGIAKWMTGAELQAYKKKYGCLPWEKISC
K:6 R:0 D:0 E:2
##########################################################################
>gi|110832851|ref|YP_691711.1| EntQ (Enterococcus faecium)
MNFLKNGIAKWMTGAELQAYKKKYGCLPWEKISC
K:6 R:0 D:0 E:2
##########################################################################
>gi|110590755|pdb|2A2B|A Chain A, Curvacin A
ARSYGNGVYCNNKKCWVNRGEATQSIIGGMISGWASGLAGM
K:2 R:2 D:0 E:1
##########################################################################
>gi|86771433|gb|ABD15215.1| plantaricin NC8 alpha peptide precursor
(Lactobacillus plantarum)
MDKFEKISTSNLEKISGGDLTTKLWSSWGYYLGKKARWNLKHPYVQF
K:7 R:1 D:2 E:2
##########################################################################
>gi|1699348|gb|AAB37479.1| dextranicin 24, Dex-24 = bacteriocin
{N-terminal} (Leuconostoc mesenteroides, ssp. dextranicum, J24,
Peptide Partial, 19 aa)
KGVLGWLSMASSALTGPQQ
K:1 R:0 D:0 E:0
##########################################################################
>gi|558004|gb|AAB31295.1| curvaticin FS47 = bacteriocin {N-terminal}
(Lactobacillus curvatus, FS47, Peptide Partial, 38 aa)
YTAKQCLQAIGSCGIAGTGAGAAGGPAGAFVGAXVVXI
K:1 R:0 D:0 E:0
##########################################################################
>gi|451255|gb|AAB28297.1| bavaricin A = bacteriocin (Lactobacillus
bavaricus, MI401, Peptide, 41 aa)
KYYGNGVHXGKHSXTVDWGTAIGNIGNNAAANXATGXNAGG
K:2 R:0 D:1 E:0
##########################################################################
>gi|257178|gb|AAB23576.1| acidocin 8912 = bacteriocin {N-terminal}
(Lactobacillus acidophilus, TK8912, Peptide Partial, 24 aa)
KTHYPTNAXKSLRKGFXESLRXTD
K:3 R:2 D:1 E:1
##########################################################################
>gi|250437|gb|AAB22371.1| 1actococcin A immunity (Lactococcus lactis)
FITSSKASNKNLGGGLIMSWGRLF
K:2 R:1 D:0 E:0
##########################################################################
>gi|254563|gb|AAB23090.1| lactococcin G peptide beta = bacteriocin
(Lactococcus lactis, LMG 2081, Peptide, 35 aa)
KKWGWLAWVDPAYEFIKGFGKGAIKEGNKDKWKNI
K:8 R:0 D:2 E:2
##########################################################################
>gi|254561|gb|AAB23088.1| lactococcin G peptide alpha 1 = bacteriocin
(Lactococcus lactis, LMG 2081, Peptide, 39 aa)
GTWDDIGQGIGRVAYWVGKAMGNMSDVNQASRINRKKKH
K:4 R:3 D:3 E:0
##########################################################################
>gi|87080648|dbj|BAE79270.1| pediocin PA-1 (synthetic construct)
KYYGNGVTCGKHSCSVDWGKATTCIINNGAMAWATGGHQGNHKC
K:4 R:0 D:1 E:0
##########################################################################
>gi|10955254|ref|NP_052370.1| cloacin lysis protein (Escherichia coli)
MKKAKAIFIFILIVSGFLLVACQANY1RDVQGGTVAPSSSSELTGIAVQ
K:3 R:1 D:1 E:1
##########################################################################
>gi|77371509|gb|ABA68548.1| Sequence 90 from patent U.S. 6,946,261
KYYGNGVHCTKSGCSVNWGEAFSAGVHRLANGGNGFW
K:2 R:1 D:0 E:1
##########################################################################
>gi|1217686|gb|AAB35815.1| plantaricin S beta chain = bacteriocin
{N-terminal} (Lactobacillus plantarum, LPCO10, Peptide Partial, 24 aa)
KKKKQSWYAAAGDAIVSFGEGFLN
K:4 R:0 D:1 E:1
##########################################################################
>gi|l217685|gb|AAB35814.1| plantaricin S alpha chain = bacteriocin
{N-terminal} (Lactobacillus plantarum, LPCO10, Peptide Partial, 26 aa)
XNKLAYNMGWYAGXATIFGLAAXALL
K:1 R:0 D:0 E:0
##########################################################################
>gi|42321|emb|CAA28145.1| unnamed protein product (Escherichia coli)
MKKAKAIFLFILIVSGFLLVACQANYIRDVQGGTVAPSSSSELTGIAVQ
K:3 R:1 D:1 E:1
##########################################################################
>gi|21355060|dbj|BAC00781.1| enterocin immunity protein
(Enterococcus faecium)
MKNNKSFNKILELTETALATP
K:3 R:0 D:0 E:2
##########################################################################
>gi|29703960|gb|AAO96899.1| Sequence 209 from patent U.S. 6,503,881
KYYGNGVHCTKSGCSVNWGEAFSAGVHRLANGGNGFW
K:2 R:1 D:0 E:1
##########################################################################
>gi|14582241|gb|AAK69420.1|AF275938_3 piscicolin 126 induction factor
PisN precursor (Carnobacterium piscicola)
MNDKKYLKLKECSEKKLKQIQGGNKSVIKGNPASNLAQCVFSFFKKC
K:11 R:0 D:1 E:2
##########################################################################
>gi|6137611|pdb|1CW5|A Chain A, Solution Structure Of Carnobacteriocin B2
XNYGNGVSCSKTKCSVNWGQAFQERYTAGINSFVSGVASGAGSIGRRP
K:2 R:3 D:0 E:1
##########################################################################
>gi|6137597|pdb|1CW6|A Chain A, Refined Solution Structure Of Leucocin A
KYYGNGVHCTKSGCSVNWGEAFSAGVHRLANGGNGFW
K:2 R:1 D:0 E:1
##########################################################################
>gi|109895375|gb|ABG47457.1| hypothetical protein (Enterococcus hirae)
MAFYLPYLLIFVSISGSIWLIYKIFQ
K:1 R:0 D:0 E:0
##########################################################################
>gi|144964437|gb|ABP07773.1| Sequence 20 from patent U.S. 7,179,889
LSGGQXQR
K:0 R:1 D:0 E:0
##########################################################################
>gi|144964436|gb|ABP07772.1| Sequence 19 from patent U.S. 7,179,889
GXXGXGKX
K:1 R:0 D:0 E:0
##########################################################################
>gi|144964427|gb|ABP07763.1| Sequence 9 from patent U.S. 7,179,889
VPGGCTYTRSNRDVIGTCKTGSGQFRIRLDCNNAPDKT
K:2 R:4 D:3 E:0
##########################################################################
>gi|113013429|gb|ABI29857.1| enterocin P protein (Enterococcus faecium)
ATRSYGNGVYCNNSKCWVNWGEAKENIAGIVISGWASGLAGMGH
K:2 R:1 D:0 E:2
##########################################################################
>gi|113013416|gb|ABI29856.1| enterocin P protein (Enterococcus faecium)
ATRSYGNGVYCNNSKCWVNWGEAKENIAGIVISGWASGLAGMGH
K:2 R:1 D:0 E:2
##########################################################################
>gi|78523018|gb|ABB46271.1| hypothetical protein (Enterococcus faecium)
MCSRSSQEYVSRYQLLILKVDRIPFPIAFILPKKGEQLNRRTFI
K:3 R:5 D:1 E:2
##########################################################################
>gi|61678013|gb|AAX52527.1| bacteriocin-like protein
(Streptococcus gordonii)
MKEFKELSKQELEKTCGGVAMPALWFFRRQAPSGNRRSSRFSLLIL
K:4 R:5 D:0 E:4
##########################################################################
>gi|121309464|dbj|BAF44074.1| hypothetical protein (Enterococcus faecium)
MRENGQKPKRSAKKTYQAPQAKKVRVTSRKEKFLEQLLKI
K:9 R:4 D:0 E:3
##########################################################################
>gi|121309463|dbj|BAF44073.1| hypothetical protein (Enterococcus faecium)
MYLSTYYPCTPHDKWAEGLAALGIKGIIRLPGF
K:2 R:1 D:1 E:1
##########################################################################
>gi|27531741|dbj|BAC54509.1| unnamed protein product
(Staphylococcus aureus)
MKQLDIPQLLIINGGSGGNYTLPGQPKGDIKKCILSFFKNC
K:5 R:0 D:2 E:0
##########################################################################
>gi|18071178|ref|NP_542225.1| hypothetical protein pRC18_p20
(Lactobacillus casei)
MLKSIFTLLIAPVLAGIAISLFDHWLDDQGRK
K:2 R:1 D:3 E:0
##########################################################################
>gi|21702214|emb|CAD35293.1| EJ97 enterocin (Enterococcus faecalis)
MLAKIKAMIKKFPNPYTLAAKLTTYEINWYKQQYGRYPWERPVA
K:6 R:2 D:0 E:2
##########################################################################
>gi|2564257|emb|CAA75396.1| plantaricin S beta protein
(Lactobacillus plantarum)
MDKIIKFQGISDDQLNAVIGGKKKKQSWYAAAGDAIVSFGEGFLNAW
K:6 R:0 D:4 E:1
##########################################################################
>gi|599853|emb|CAA86943.1| orf4 (Lactobacillus sakei)
MKLNYIEKKQLTNKQLKLIIGGTNRNYGKPNKDIGTCIWSGFRHC
K:7 R:2 D:1 E:1
##########################################################################
>gi|75707048|gb|ABA26010.1| CopG (Streptococcus dysgalactiae subsp.
equisimilis)
MKKRLTITLSDSVLENLEKMAKEMGLSKSAMISVALENYKKGQEK
K:8 R:1 D:1 E:5
##########################################################################
>gi|111948936|gb|ABH72298.1| Sequence 3 from patent U.S. 7,034,113
FKSWSFCTPGCAKTGSFNSYCC
K:2 R:0 D:0 E:0
##########################################################################
>gi|62721688|gb|AAX94281.1| CopG (Streptococcus dysgalactiae subsp.
equisimilis)
MKKRLTITLSDSVLENLEKMAKEMGLSKSAMISVALENYKKGQEK
K:8 R:1 D:1 E:5
##########################################################################
>gi|90903985|gb|ABE02387.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903983|gb|ABE02386.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903981|gb|ABE02385.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903979|gb|ABE02384.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903977|gb|ABE02383.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903975|gb|ABE02382.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903973|gb|ABE02381.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903971|gb|ABE02380.1| competence stimulating peptide precursor
(Streptococcus mutans)
MKGTLSLKNDFKEIKTDELEIIIGGSGSLSTFFRLFNRSFTQALGK
K:5 R:2 D:2 E:3
##########################################################################
>gi|90903969|gb|ABE02379.1| competence stimulating peptide precursor
(Streptococcus mutans)
MKKTPSLKNDFKEIKTDELEIIIGGSGSLSTFFRLFNRSFTQALGKIR
K:6 R:3 D:2 E:3
##########################################################################
>gi|90903967|gb|ABE02378.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi/|0903965|gb|ABE02377.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903963|gb|ABE02376.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903961|gb|ABE02375.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903959|gb|ABE02374.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903957|gb|ABE02373.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903955|gb|ABE02372.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903953|gb|ABE02371.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903951|gb|ABE02370.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903949|gb|ABE02369.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903947|gb|ABE02368.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903945|gb|ABE02367.1| competence stimulating peptide precursor
(Streptococcus mutans)
MKKTPSLKNDFKEIKTDELEIIIGGSGSLSTFFRLFNRSFTQALGK
K:6 R:2 D:2 E:3
##########################################################################
>gi|90903943|gb|ABE02366.1| competence stimulating peptide precursor
(Streptococcus mutans)
MKKTLSLKNDFKEIKTDELEIIIGGSGSLSTFFRLFNRSFTQALGK
K:6 R:2 D:2 E:3
##########################################################################
>gi|90903941|gb|ABE02365.1| competence stimulating peptide precursor
(Streptococcus mutans)
MKKTPSLKNDFKEIKTDELEIIIGGSGSLSTFFRLFNRSFTQALGKIR
K:6 R:3 D:2 E:3
##########################################################################
>gi|90903939|gb|ABE02364.1| competence stimulating peptide precursor
(Streptococcus mutans)
MKKTLSLKNDFKEIKTDELEIITGGSGSLSTFFRLFNRSFTQA
K:5 R:2 D:2 E:3
##########################################################################
>gi|90903937|gb|ABE02363.1| competence stimulating peptide precursor
(Streptococcus mutans)
MKKTLSLKNDFKEIKTDELEIIIGGSGSLSTFFRLFNRSFTQA
K:5 R:2 D:2 E:3
##########################################################################
>gi|84569626|gb|ABC59154.1| precursor peptide Plnc8IF
(Lactobacillus plantarum)
MKNINKYTELNDQKLQSLIGGKTKTISLMSGLQVPHAFTKLLKALGGHH
K:7 R:0 D:1 E:1
##########################################################################
>gi|84569619|gb|ABC59147.1| plantaricin biosynthesis protein PlnR
(Lactobacillus plantarum)
MLNKTINIlKKYPVRSLLVVLIVVFAIYVISDPSIISSFNQGLSDGTAGR
K:3 R:2 D:2 E:0
##########################################################################
>gi|86771439|gb|ABD15221.1| plantaricin A precursor peptide,
induction factor (Lactobacillus plantarum)
MKIQIKSMKQLSNKEMQKIVGGKSSAYSLQMGATAIKQVKKLFKKWGW
K:11 R:0 D:0 E:1
##########################################################################
>gi|86771435|gb|ABD15217.1| unknown (Lactobacillus plantarum)
MLNKTINIIKKYPVRSLLVVLIVVFAIYVISDGAAGR
K:3 R:2 D:1 E:0
##########################################################################
>gi|1000432|gb|AAB34888.1| pediocin L50 {C-terminal}
(Pediococcus acidilactici, Peptide Partial, 41 aa)
MGAIAKLVAKFGXXIVVKYYKQIMQFIGQGVTINXIPLIXF
K:4 R:0 D:0 E:0
##########################################################################
>gi|1881836|gb|AAB49524.1| acidocin J1132 beta peptide {N-terminal}
(Lactobacillus acidophilus, JCM 1132, Peptide Partial, 24 aa)
GNPKVAHCASQIGRSTAWGAVSGA
K:1 R:1 D:0 E:0
##########################################################################
>gi|1881835|gb|AAB49523.1| acidocin J1132 alpha peptide {N-terminal}
(Lactobacillus acidophilus, JCM 1132, Peptide Partial, 23 aa)
NPKVAHCASQIGRSTAWGAVSGA
K:1 R:1 D:0 E:0
##########################################################################
>gi|19718342|ref|NP_604414.1| acidocin 8912 (Lactobacillus acidophilus)
MISSHQKTLTDKELALISGGKTHYPTNAWKSLWKGFWESLRYTDGF
K:5 R:1 D:2 E:2
##########################################################################
>gi|50812300|ref|YP_054594.1| bacteriocin-like product (Bacillus subtilis
subsp. subtilis str. 168)
MKLPVQQVYSVYGGKDLPKGHSHSTMPFLSKLQFLTKIYLLDIHTQPFFI
K:5 R:0 D:2 E:0
##########################################################################
>gi|73486986|gb|AAZ76605.1| BhtB (Streptococcus ratti)
MWGRILAFVAKYGTKAVQWAWKNKWFLLSLGEAVFDYIRSIWGG
K:4 R:2 D:1 E:1
##########################################################################
>gi|72069115|dbj|BAE17145.1| lcnB homolog (Lactococcus lactis subsp.
cremoris)
ELAEVNGGSLQYVMSAGPYTWYKDTRTGKTICKQTIDTASYT
K:3 R:1 D:2 E:2
##########################################################################
>gi|140801|sp|P22296|YHV4_LACHE Hypothetical protein in hlv 3′region
(ORF4)
MHNSIAYDKDGNSTGQKYYAYG
K:2 R:0 D:2 E:0
##########################################################################
>gi|45826080|gb|AAS77688.1| ErmBL (Shuttle vector pLPV111)
MLVFQMRNVDKTSTVLKQTKNSDYADK
K:4 R:1 D:3 E:0
##########################################################################
>gi|62769895|gb|AAY00813.1| Sequence 12 from patent U.S. 6,855,518
KYYGNGVSCNSHGCSVNWGQAWTCGVNHLANGGHGVC
K:1 R:0 D:0 E:0
##########################################################################
>gi|452406|emb|CAA53069.1| precursor for plantaricin A
(Lactobacillus plantarum)
MKIQIKGMKQLSNKEMQKIVGGKSSAYSLQMGATAIKQVKKLFKKWGW
K:11 R:0 D:0 E:1
##########################################################################
>gi|32812396|emb|CAD97584.1| circularin immunity protein
(Clostridium beijerinckii)
MNKKKILIYAILFLIYIILFLTYNNSIFRIILVVSLGFLSSIISKLQIK
K:5 R:1 D:0 E:0
##########################################################################
>gi|7514358|pir||A58718 carnocin U149 - Carnobacterium sp. (fragment)
GSEIQPR
K:0 R:1 D:0 E:1
##########################################################################
>gi|482590|pir||A49779 lactacin F - Lactobacillus acidophilus (fragment)
RNNWQTNVGGAVGXAMIGATVGGTI
K:0 R:1 D:0 E:0
##########################################################################
>gi|45826075|gb|AAS77684.1| ErmBL (Shuttle vector pELS200)
MLVFQMRNVDKTSTVLKQTKNSDYADK
K:4 R:1 D:3 E:0
##########################################################################
>gi|37783311|gb|AAP44562.1| IP-TX (Lactobacillus sakei)
MTNRKTLPKEELKKIKGGTPGGFDIISGGPHVAQDVLNAIKDFFK
K:7 R:1 D:3 E:2
##########################################################################
>gi|6176540|gb|AAF05610.1|AF190857_3 Cex (Klebsiella pneumoniae)
MKKVKTIFLFILIASGFLLVACQANYNRDVQGGTVAPSSSSELTGIAVQ
K:3 R:1 D:1 E:1
##########################################################################
>gi|21541353|gb|AAM61781.1|AF408405_9 AbpIP (Lactobacillus salivarius
subsp. salivarius)
MKFEVLTEKKLQKIAGGATKKGGFKRWQCIFTFFGVCK
K:8 R:1 D:0 E:2
##########################################################################
>gi|21541348|gb|AAM61776.1|AF408405_4 bacteriocin-like prepeptide
(Lactobacillus salivarius subsp. salivarius)
MLKKLWNIWLDGGLIRGRKRYVIIPIIFAIFLPLSMWLSDNEGMSYLDYI
K:3 R:3 D:3 E:1
##########################################################################
>gi|19570487|dbj|BAB86322.1| acidocin 8912 (Lactobacillus acidophilus)
MISSHQKTLTDKELALISGGKTHYPTNAWKSLWKGFWESLRYTDGF
K:5 R:1 D:2 E:2
##########################################################################
>gi|17986222|gb|AAL54832.1| hypothetical protein (Lactobacillus casei)
MLKSIFTLLIAPVLAGIAISLFDHWLDDQGRK
K:2 R:1 D:3 E:0
##########################################################################
>gi|14861186|gb|AAK73555.1|AF241888_8 AurD (Staphylococcus aureus)
MGAVIKVGAKVIGWGAASGAGLYGLEKILKK
K:5 R:0 D:0 E:1
##########################################################################
>gi|14861185|gb|AAK73554.1|AF241888_7 AurC (Staphylococcus aureus)
MGALIKTGAKIIGSGAAGGLGTYIGHKILGK
K:4 R:0 D:0 E:0
##########################################################################
>gi|14861184|gb|AAK73553.1|AF241888_6 AurB (Staphylococcus aureus)
MGAVAKFLGKAALGGAAGGATYAGLKKIFG
K:4 R:0 D:0 E:0
##########################################################################
>gi|14861183|gb|AAK73552.1|AF241888_5 AurA (Staphylococcus aureus)
MGKLAIKAGKIIGGGIASALGWAAGEKAVGK
K:5 R:0 D:0 E:1
##########################################################################
>gi|9454298|gb|AAF87750.1|AF278540_2 unknown (Clostridium botulinum)
MEFKNKQRMYREFFMTLKESFKFSSKKRYI
K:6 R:3 D:0 E:3
##########################################################################
>gi|5441254|dbj|BAA82352.1| ORF3 (Lactobacillus gasseri)
MLDKNTDLQRAIFHIKQDINLYSVVYGFKLPET
K:3 R:1 D:3 E:1
##########################################################################
>gi|6941874|gb|AAF32255.1| unknown protein (Lactococcus lactis)
MKKKFQDSISNSVYKYRVLSRLSQQD
K:4 R:2 D:2 E:0
##########################################################################
>gi|149556|gb|AAA63275.1| ORF4 (Lactobacillus helveticus)
MHNSIAYDKDGNSTGQKYYAYGS
K:2 R:0 D:2 E:0
##########################################################################
>gi|5616079|gb|AAD45619.1|AF080265_3 unknown (Lactococcus lactis subsp.
lactis)
MLIKVLEKKSYLRMLQLTLIEIVYISLWHPMVQGKQPFLR
K:4 R:2 D:0 E:2
##########################################################################
>gi|2735687|gb|AAB93967.1| inducing peptide preprotein (Lactobacillus
sakei)
MMIFKKLSEKELQKINGGMAGNSSNFIHKIKQIFTHR
K:6 R:1 D:0 E:2
##########################################################################
>gi|972708|gb|AAB81301.1| ORF-3; unknown function (Carnobacterium
piscicola)
MKNFFKKNNMLYRFFAVIGLIFGGWALFNIAMFIGRSIGSLF
K:3 R:2 D:0 E:0
##########################################################################
>gi|1041118|dbj|BAA11198.1| iPD1 (Enterococcus faecalis)
MKQQKKHIAALLFALILTLVS
K:3 R:0 D:0 E:0
##########################################################################
>gi|1088253|gb|AAA87233.1| ORF; putative
MEPNKNKDLGLAALKILAQYHNISVNPEELKHKFDL
K:5 R:0 D:2 E:3
##########################################################################
>gi|388270|gb|AAA72025.1| traA
KRLE
K:1 R:1 D:0 E:1
##########################################################################
>gi|388268|gb|AAA72023.1| traB
QDDISSIKCIYKNRLLKVGL1FVLASAGGAIGNIIGGIELFKNLI
K:4 R:1 D:2 E:1
##########################################################################
>gi|475430|gb|AAA67127.1| repA gene product
KKSNSNTPGVITHNWVENQ
K:2 R:0 D:0 E:1

TABLE 3
Non-natural amino acids
Tryptophan variants

| | |
|---|---|
| 2. | DL-7-azatryptophan |
| 3. | β-(3-benzothienyl)-L-alanine |
| 4. | β-(3-benzothienyl)-D-alanine |
| 5. | 5-benzyloxy-DL-tryptophan |
| 6. | 7-benzyloxy-DL-tryptophan |
| 7. | 5-bromo-DL-tryptophan |
| 8. | 5-fluoro-DL-tryptophan |
| 9. | 6-fluoro-DL-tryptophan |
| 10. | 5-hydroxy-L-tryptophan |
| 11. | 5-hydroxy-DL-tryptophan |
| 12. | 5-methoxy-DL-tryptophan |
| 13. | α-methyl-DL-tryptophan |
| 14. | 1-methyl-DL-tryptophan |
| 15. | 5-methyl-DL-tryptophan |
| 16. | 6-methyl-DL-tryptophan |
| 17. | 7-methyl-DL-tryptophan |
| 18. | D-1,2,3,4-tetrahydronorharman-3-carboxylic acid |
| 19. | DL-6-methoxy-1,2,3,4-tetrahydronorharman-1-carboxylic acid |
| 20. | 5-Hydroxytryptophan: 2-Amino 3-(5-hydroxyindolyl)-propionic acid |
| 21. | L-Neo-Tryptophan |
| 22. | D-Neo-Tryptophan |

Phenylalanine and Tyrosine variants

| | |
|---|---|
| 24. | 4-aminomethyl)-L-phenylalanine |
| 25. | 4-aminomethyl)-D-phenylalanine |
| 26. | 4-amino-L-phenylalanine |
| 27. | 4-amino-D-phenylalanine |
| 28. | 3-amino-L-tyrosine |
| 29. | 4-bromo-L-phenylalanine |
| 30. | 4-bromo-D-phenylalanine |
| 31. | 4-bis(2-chloroethyl)amino-L-phenylalanine |
| 32. | 2-chloro-L-phenylalanine |
| 33. | 2-chloro-D-phenylalanine |
| 34. | 4-chloro-L-phenylalanine |
| 35. | 4-chloro-D-phenylalanine |
| 36. | 3-chloro-L-tyrosine |
| 37. | 3,4-dichloro-L-phenylalanine |
| 38. | 3,4-dichloro-D-phenylalanine |
| 39. | 3,4-difluoro-L-phenylalanine |
| 40. | 3,4-difluoro-D-phenylalanine |
| 41. | 3,4-dihydroxy-L-phenylalanine |
| 42. | 3,5-diiodo-L-thyronine |
| 43. | 3,5-diiodo-D-tyrosine |
| 44. | 3,4-dimethoxy-L-phenylalanine |
| 45. | 3,4-dimethoxy-DL-phenylalanine |
| 46. | O-ethyl-L-tyrosine |
| 47. | O-ethyl-D-tyrosine |
| 48. | 2-fluoro-L-phenylalanine |
| 49. | 2-fluoro-D-phenylalanine |
| 50. | 4-fluoro-L-phenylalanine |
| 51. | 4-fluoro-D-phenylalanine |
| 52. | 3-fluoro-DL-tyrosine |
| 53. | L-homophenylalanine |
| 54. | D-homophenylalanine |
| 55. | 2-hydroxy-3-methyl-L-phenylalanine |
| 56. | 2-hydroxy-3-methyl-D-phenylalanine |
| 57. | 2-hydroxy-3-methyl-DL-phenylalanine |
| 58. | 2-hydroxy-4-methyl-L-phenylalanine |
| 59. | 2-hydroxy-4-methyl-D-phenylalanine |
| 60. | 2-hydroxy-4-methyl-DL-phenylalanine |
| 61. | 2-hydroxy-5-methyl-L-phenylalanine |
| 62. | 2-hydroxy-5-methyl-D-phenylalanine |
| 63. | 2-hydroxy-5-methyl-DL-phenylalanine |
| 64. | β-hydroxy-DL-phenylalanine (DL-threo-3-phenylserine) |
| 65. | 7-hydroxy-(S)-1,2,3,4-tetrahydroisoquinoline-3-carboxylic acid (hydroxy-Tic-OH) |
| 66. | 7-hydroxy-(R)-1,2,3,4-tetrahydroisoquinoline-3-carboxylic acid (hydroxy-D-Tic- |
| | OH) |
| 67. | 4-iodo-L-phenylalanine |
| 68. | 4-iodo-D-phenylalanine |
| 69. | 3-iodo-L-tyrosine |
| 70. | α-methyl-3-methoxy-DL-phenylalanine |
| 71. | α-methyl-4-methoxy-L-phenylalanine |
| 72. | α-methyl-4-methoxy-DL-phenylalanine |
| 73. | α-methyl-L-phenylalanine |
| 74. | α-methyl-D-phenylalanine |
| 75. | β-methyl-DL-phenylalanine |
| 76. | α-methyl-DL-tyrosine |
| 77. | O-methyl-L-tyrosine |
| 78. | O-methyl-D-tyrosine |
| 79. | 4-nitro-L-phenylalanine |
| 80. | 4-nitro-D-phenylalanine |
| 81. | 3-nitro-L-tyrosine |
| 82. | (S)-1,2,3,4-tetrahydroisoquinoline-3-carboxylic acid (L-Tic-OH) |
| 83. | (R)-1,2,3,4-tetrahydroisoquinoline-3-carboxylic acid (D-Tic-OH) |
| 84. | L-thyronine |
| 85. | DL-thyronine |
| 86. | L-thyroxine |
| 87. | D-thyroxine |
| 88. | 2,4,5-trihydroxy-DL-phenylalanine |
| 89. | 3,5,3′-triiodo-L-thyronine |
| 90. | DL-m-tyrosine |
| 91. | DL-o-tyrosine |
| 92. | 2-(trifluoromethyl)-L-phenylalanine |
| 93. | 2-(trifluoromethyl)-D-phenylalanine |
| 94. | 2-cyano-L-phenylalanine |
| 95. | 2-cyano-D-phenylalanine |
| 96. | 2-methyl-L-phenylalanine |
| 97. | 2-methyl-D-phenylalanine |
| 98. | 3-(trifluoromethyl)-L-phenylalanine |
| 99. | 3-(trifluoromethyl)-D-phenylalanine |
| 100. | 3-cyano-L-phenylalanine |
| 101. | 3-cyano-D-phenylalanine |
| 102. | 3-fluoro-L-phenylalanine |
| 103. | fluoro-D-phenylalanine |
| 104. | 3-methyl-L-phenylalanine |
| 105. | 3-methyl-D-phenylalanine |
| 106. | 4-benzoyl-L-phenylalanine |
| 107. | 4-benzoyl-D-phenylalanine |
| 108. | 4-(trifluoromethyl)-L-phenylalanine |
| 109. | 4-(trifluoromethyl)-D-phenylalanine |
| 110. | 4-cyano-L-phenylalanine |
| 111. | 4-cyano-D-phenylalanine |
| 112. | 4-methyl-L-phenylalanine |
| 113. | 4-methyl-D-phenylalanine |
| 114. | 2,4-dichloro-L-phenylalanine |
| 115. | 2,4-dichloro-D-phenylalanine |
| 116. | 3,5-diiodo-L-tyrosine OSu |

Arginine and Lysine variants

| | |
|---|---|
| 118. | L-2-amino-3-guanidinopropionic acid |
| 119. | L-2-amino-3-ureidopropionic acid (Albizziin) |
| 120. | L-citrulline |
| 121. | DL-citrulline |
| 122. | 2,6-diaminoheptanedioic acid (mixture of isomers) |
| 123. | N-ω,ω-dimethyl-L-arginine (symmetrical) |
| 124. | N-ε,ε-dimethyl-L-lysine hydrochloride salt |
| 125. | α-methyl-DL-ornithine |
| 126. | N-ω-nitro-L-arginine |
| 127. | N-ω-nitro-D-arginine |
| 128. | N-δ-benzyloxycarbonyl-L-ornithine |
| 129. | (N-δ-)-L-ornithine |
| 130. | (N-δ-)-D-ornithine |
| 131. | (N-δ-1-(4,4-dimethyl-2,6-dioxocyclohex-1-ylidene)ethyl)-D-ornithine (D-Orn- |
| | (Dde)-OH) |
| 132. | L-ornithine (Orn( )-OH) |
| 133. | (N-d-4-methyltrityl)-L-ornithine (Orn(Mtt)-OH) |
| 134. | (N-d-4-methyltrityl)-D-ornithine (D-Orn(Mtt)-OH) |

Proline variants

| | |
|---|---|
| 136. | cis-4-amino-L-proline methyl ester hydrochloride salt |
| 137. | trans-4-amino-L-proline methyl ester hydrochloride salt |
| 138. | (S)-azetidine-2-carboxylic acid |
| 139. | trans-4-cyano-L-proline |
| 140. | cis-4-cyano-L-proline methyl ester |
| 141. | trans-4-cyano-L-proline methyl ester |
| 142. | 3,4-dehydro-L-proline |
| 143. | (R)-5,5-dimethylthiazolidine-4-carboxylic acid |
| 144. | (4S,2RS)-2-ethylthiazolidine-4-carboxylic acid |
| 145. | trans-4-fluoro-L-proline |
| 146. | (2S,3S)-3-hydroxypyrrolidine-2-carboxylic acid (trans-3-hydroxy-L-proline) |
| 147. | (2S,4S)-(−)-4-hydroxypyrrolidine-2-carboxylic acid (cis-4-hydroxy-L-proline) |
| 148. | (2S,4R)-(−)-4-hydroxypyrrolidine-2-carboxylic acid (trans-4-hydroxy-L-proline) |
| 149. | (2R,4R)-(+)-4-hydroxypyrrolidine-2-carboxylic acid (cis-4-hydroxy-D-proline) |
| 150. | (2S,4R)-(−)-4-t-butoxypyrrolidine-2-carboxylic acid (trans-4-t-butoxy-L-proline) |
| 151. | (2S,5RS)-5-methylpyrrolidine-2-carboxylic acid |
| 152. | (4S,2RS)-2-methylthiazolidine-4-carboxylic acid |
| 153. | (2S,3R)-3-phenylpyrrolidine-2-carboxylic acid |
| 154. | (4S,2RS)-2-phenylthiazolidine-4-carboxylic acid |
| 155. | (S)-thiazolidine-2-carboxylic acid |
| 156. | (R)-thiazolidine-2-carboxylic acid |
| 157. | (S)-thiazolidine-4-carboxylic acid |
| 158. | (R)-thiazolidine-4-carboxylic acid (L-thioproline) |
| 159. | α-allyl-DL-proline |
| 160. | α-benzyl-DL-proline |
| 161. | α-(2-bromobenzyl)-DL-proline |
| 162. | α-(4-bromobenzyl)-DL-proline |
| 163. | α-(2-chlorobenzyl)-DL-proline |
| 164. | α-(3-chlorobenzyl)-DL-proline |
| 165. | α-(diphenylmethyl)-DL-proline |
| 166. | α-(4-fluorobenzyl)-DL-proline |
| 167. | α-methyl-DL-proline |
| 168. | α-(4-methylbenzyl)-DL-proline |
| 169. | α-(1-naphthylmethyl)-DL-proline |
| 170. | α-propyl-DL-proline |
| 171. | 4-benzyl-L-pyroglutamic |
| 172. | 4-(2-bromobenzyl)-L-pyroglutamic acid benzyl ester |
| 173. | 4-(4-bromobenzyl)-L-pyroglutamic acid benzyl ester |
| 174. | 4-(4-methylbenzyl)-L-pyroglutamic acid benzyl ester |

Miscellaneous Hetercyclic Amino Acids

| | |
|---|---|
| 176. | α-amino-3-chloro-4,5-dihydro-5-isoxazoleacetic acid |
| 177. | 2-amino-α-(methoxyimino)-4-thiazoleacetic acid (predominantly syn) |
| 178. | 5-aminoorotic acid |
| 179. | 2-aminopyridyl-3-carboxylic acid (2-aminonicotinic acid) |
| 180. | 6-aminopyridyl-3-carboxylic acid (6-aminonicotinic acid) |
| 181. | 2-aminothiazole-4-acetic acid |
| 182. | (S)-azetidine-2-carboxylic acid |
| 183. | azetidine-3-carboxylic acid |
| 184. | 4-carboxymethylpiperazine |
| 185. | 4-carboxymethylpiperazine |
| 186. | 2-carboxypiperazine |
| 187. | 3-carboxypiperidine |
| 188. | indoline-2-carboxylic acid |
| 189. | L-mimosine |
| 190. | 4-phenylpiperidine-4-carboxylic acid |
| 191. | (S)-(−)-piperidine-2-carboxylic acid (L-(−)-pipecolic acid) |
| 192. | (R)-(+)-piperidine-2-carboxylic acid (D-(+)-pipecolic acid) |
| 193. | (RS)-piperidine-2-carboxylic acid (DL-pipecolic acid) |
| 194. | piperidine-4-carboxylic acid (isonipecotic acid) |

Analogs of Alanine, Glycine, Valine, and Leucine

| | |
|---|---|
| 196. | 3-(2-furyl)-D-Ala-OH |
| 197. | 3-cyclopentyl-DL-Ala-OH |
| 198. | 3-(4-quinolyl)-DL-Ala-OH |
| 199. | 3-(4-quinolyl)-DL-Ala-OH dihydrochloride dihydrate |
| 200. | 3-(2-quinolyl)-DL-Ala-OH |
| 201. | 3-(2-quinoxalyl)-DL-Ala-OH |
| 202. | α-allyl-L-alanine |
| 203. | L-allylglycine |
| 204. | L-allylglycine dicyclohexylammonium salt |
| 205. | D-allylglycine |
| 206. | D-allylglycine dicyclohexylammonium salt |
| 207. | L-α-aminobutyric acid (Abu-OH) |
| 208. | D-α-aminobutyric acid (D-Abu-OH) |
| 209. | DL-β-aminobutyric acid (DL-β-Abu-OH) |
| 210. | γ-aminobutyric acid (γ-Abu-OH) |
| 211. | α-aminoisobutyric acid (Aib-OH) |
| 212. | DL-β-aminoisobutyric acid (DL-β-Aib-OH) |
| 213. | Di-N-α-aminomethyl-L-alanine |
| 214. | 2-amino-4,4,4-trifluorobutyric acid |
| 215. | 3-amino-4,4,4-trifluorobutyric acid |
| 216. | β-(3-benzothienyl)-L-alanine |
| 217. | β-(3-benzothienyl)-D-alanine |
| 218. | t-butyl-L-alanine |
| 219. | t-butyl-D-alanine |
| 220. | L-t-butylglycine |
| 221. | D-t-butylglycine |
| 222. | β-cyano-L-alanine |
| 223. | β-cyclohexyl-L-alanine (Cha-OH) |
| 224. | β-cyclohexyl-D-alanine (D-Cha-OH) |
| 225. | L-cyclohexylglycine (Chg-OH) |
| 226. | D-cyclohexylglycine (D-Chg-OH) |
| 227. | β-cyclopentyl-DL-alanine |
| 228. | β-cyclopenten-1-yl-DL-alanine |
| 229. | β-cyclopropyl-L-alanine |
| 230. | cyclopropyl-DL-phenylglycine |
| 231. | DL-dehydroarmentomycin |
| 232. | 4,5-dehydro-L-leucine |
| 233. | L-α,γ-diaminobutyric acid (Dab-OH) |
| 234. | D-α,γ-diaminobutyric acid (D-Dab-OH) |
| 235. | Di-L-α,γ-diaminobutyric acid (Dab( )-OH) |
| 236. | Di-D-α,γ-diaminobutyric acid (D-Dab( )-OH) |
| 237. | (N-γ-allyloxycarbonyl)-L-α,γ-diaminobutyric acid (Dab(Aloc)-OH) |
| 238. | (N-γ-)-L-α,γ-diaminobutyric acid (Dab( )-OH) |
| 239. | (N-γ-1-(4,4-dimethyl-2,6-dioxocyclohex-1-ylidene)ethyl)-L-α,γ-diaminobutyric |
| | acid (Dab(Dde)-OH) |
| 240. | (N-γ-4-methyltrityl)-L-α,γ-diaminobutyric acid (Dab(Mtt)-OH) |
| 241. | (N-γ-)-D-α,γ-diaminobutyric acid (D-Dab( )-OH) |
| 242. | (N-γ-1-(4,4-dimethyl-2,6-dioxocyclohex-1-ylidene)ethyl)-D-α,γ-diaminobutyric |
| | acid (D-Dab(Dde)-OH) |
| 243. | (N-γ-4-methyltrityl)-D-α,γ-diaminobutyric acid (D-Dab(Mtt)-OH) |
| 244. | L-α,β-diaminopropionic acid (Dap-OH) |
| 245. | D-α,β-diaminopropionic acid (D-Dap-OH) |
| 246. | Di-L-α,β-diaminopropionic acid (Dap( )-OH) |
| 247. | Di-D-α,β-diaminopropionic acid (D-Dap( )-OH) |
| 248. | (N-β-allyloxycarbonyl)-L-α,β-diaminopropionic acid (Dap(Aloc)-OH) |
| 249. | (N-β-)-L-α,β-diaminopropionic acid (Dap( )-OH) |
| 250. | β-(1-naphthyl)-D-alanine (D-1-Nal-OH) |
| 251. | β-(2-naphthyl)-L-alanine (2-Nal-OH) |
| 252. | β-(2-naphthyl)-D-alanine (D-2-Nal-OH) |
| 253. | L-phenylglycine (Phg-OH) |
| 254. | D-phenylglycine (D-Phg-OH) |
| 255. | L-propargylglycine |
| 256. | L-propargylglycine dicyclohexylammonium salt |
| 257. | D-propargylglycine |
| 258. | D-propargylglycine dicyclohexylammonium salt |
| 259. | β-(2-pyridyl)-L-alanine (L-2-pyridylalanine) |
| 260. | β-(2-pyridyl)-D-alanine (D-2-pyridylalanine) |
| 261. | β-(3-pyridyl)-L-alanine (L-3-pyridylalanine) |
| 262. | β-(3-pyridyl)-D-alanine (D-3-pyridylalanine) |
| 263. | β-(4-pyridyl)-L-alanine (L-4-pyridylalanine) |
| 264. | β-(4-pyridyl)-D-alanine (D-4-pyridylalanine) |
| 265. | β-(2-thienyl)-L-alanine (Thi-OH) |
| 266. | β-(2-thienyl)-D-alanine (D-Thi-OH) |
| 267. | L-(2-thienyl)glycine |
| 268. | D-(2-thienyl)glycine |
| 269. | L-(3-thienyl)glycine |
| 270. | D-(3-thienyl)glycine |
| 271. | 5,5,5-trifluoro-DL-leucine |
| 272. | 4,4,4-trifluoro-DL-valine |
| 273. | L-2-amino-3-(dimethylamino)propionic acid (aza-L-leucine) |
| 274. | DL-2-amino-3-(dimethylamino)propionic acid (aza-DL-leucine) |
| 275. | (N-β-1-(4,4-dimethyl-2,6-dioxocyclohex-1-ylidene)ethyl)-L-α,β-diaminopropionic |
| | acid (Dap(Dde)-OH) |
| 276. | (N-β-(2,4-dinitrophenyl))-L-α,β-diaminopropionic acid (Dap(Dnp)-OH) |
| 277. | (N-β-4-methyltrityl)-L-α,β-diaminopropionic acid (Dap(Mtt)-OH) |
| 278. | (N-β-)-L-α,β-diaminopropionic acid (Dap( )-OH) |
| 279. | (N-β-)-D-α,β-diaminopropionic acid (D-Dap( )-OH) |
| 280. | (N-β-1-(4,4-dimethyl-2,6-dioxocyclohex-1-ylidene)ethyl)-D-α,β-diaminopropionic |
| | acid (D-Dap(Dde)-OH) |
| 281. | 2,5-dihydro-D-phenylglycine |
| 282. | 2,4-dinitro-DL-phenylglycine |
| 283. | 2-fluoro-DL-phenylglycine |
| 284. | 4-fluoro-L-phenylglycine |
| 285. | 4-fluoro-D-phenylglycine |
| 286. | 3-fluoro-DL-valine |
| 287. | 4-hydroxy-D-phenylglycine |
| 288. | α-methyl-DL-leucine |
| 289. | β-(1-naphthyl)-L-alanine (1-Nal-OH) |
| 290. | β-(1-naphthyl)-D-alanine (D-1-Nal-OH) |

Analogs of Benzoic Acid

| | |
|---|---|
| 292. | 2-amino-4-fluorobenzoic acid |
| 293. | 2-amino-5-fluorobenzoic acid |
| 294. | 2-amino-6-fluorobenzoic acid |
| 295. | 2-amino-5-iodobenzoic acid |
| 296. | 2-amino-3-methoxybenzoic acid |
| 297. | 2-amino-5-methoxybenzoic acid |
| 298. | 3-amino-4-methoxybenzoic acid |
| 299. | 4-amino-3-methoxybenzoic acid |
| 300. | 2-amino-3-methylbenzoic acid |
| 301. | 2-amino-5-methylbenzoic acid |
| 302. | 2-amino-6-methylbenzoic acid |
| 303. | 3-amino-2-methylbenzoic acid |
| 304. | 3-amino-4-methylbenzoic acid |
| 305. | 4-amino-3-methylbenzoic acid |
| 306. | 3-aminomethylbenzoic acid (Mamb-OH) |
| 307. | 4-aminomethylbenzoic acid (Pamb-OH) |
| 308. | 2-amino-3,4,5-trimethoxybenzoic acid |
| 309. | Di-3,4-diaminobenzoic acid |
| 310. | Di-3,5-diaminobenzoic acid |
| 311. | 4-methylaminobenzoic acid |
| 312. | 5-acetamido-2-aminobenzoic acid (5-acetamidoanthranilic acid) |
| 313. | 2-aminobenzene-1,4-dicarboxylic acid |
| 314. | 3-aminobenzene-1,2-dicarboxylic acid |
| 315. | 2-aminobenzoic acid (2-Abz-OH) |
| 316. | 3-aminobenzoic acid (3-Abz-OH) |
| 317. | 4-aminobenzoic acid (4-Abz-OH) |
| 318. | 2-(2-aminobenzoyl)benzoic acid |
| 319. | 2-amino-5-bromobenzoic acid |
| 320. | 2-amino-4-chlorobenzoic acid |
| 321. | 2-amino-5-chlorobenzoic acid |
| 322. | 2-amino-6-chlorobenzoic acid |
| 323. | 3-amino-4-chlorobenzoic acid |
| 324. | 4-amino-2-chlorobenzoic acid |
| 325. | 5-amino-2-chlorobenzoic acid |
| 326. | 2-amino-4,5-dimethoxybenzoic acid |
| 327. | 2-amino-3,5-dimethylbenzoic acid |
| 328. | 2-amino-4-fluorobenzoic acid |

Miscellaneous Aromatic Amino Acids

| | |
|---|---|
| 330. | Di-2-amino-3-(2-aminobenzoyl)propionic acid |
| 331. | 4-aminocinnamic acid (predominantly trans) |
| 332. | 4-aminohippuric acid |
| 333. | 3-amino-2-naphthoic acid |
| 334. | 4-aminooxanilic acid |
| 335. | (3-aminophenyl)acetic acid |
| 336. | (4-aminophenyl)acetic acid |
| 337. | 4-(4-aminophenyl)butanoic acid |
| 338. | 3-amino-3-phenylpropionic acid |
| 339. | (4-aminophenylthio)acetic acid |
| 340. | (2R,3S)-2-amino-3-(phenylthio)butanoic acid |
| 341. | Analogs of Cysteine and Methionine |
| 342. | S-acetamidomethyl-L-penicillamine |
| 343. | S-acetamidomethyl-D-penicillamine |
| 344. | S-(2-aminoethyl)-L-cysteine |
| 345. | S-benzyl-L-cysteine |
| 346. | S-benzyl-D-cysteine |
| 347. | S-benzyl-DL-homocysteine |
| 348. | L-buthionine |
| 349. | L-buthioninesulfoximine |
| 350. | DL-buthioninesulfoximine |
| 351. | S-n-butyl-L-cysteine |
| 352. | S-t-butyl-L-cysteine |
| 353. | S-t-butyl-D-cysteine |
| 354. | S-carbamoyl-L-cysteine |
| 355. | S-carboxyethyl-L-cysteine |
| 356. | S-carboxymethyl-L-cysteine |
| 357. | L-cysteic acid |
| 358. | S-diphenylmethyl-L-cysteine |
| 359. | L-ethionine (2-amino-4-(ethyl(thio)butyric acid) |
| 360. | D-ethionine (D-2-amino-4-(ethyl(thio)butyric acid) |
| 361. | S-ethyl-L-cysteine |
| 362. | S-trityl-L-homocysteine |
| 363. | Di-L-homocystine |
| 364. | DL-methionine methylsulfonium chloride |
| 365. | S-4-methoxybenzyl-L-penicillamine |
| 366. | S-4-methoxybenzyl-L-penicillamine (Pen(4-MeOBzl)-OH) |
| 367. | S-4-methylbenzyl-L-penicillamine dicyclohexylammonium salt (Pen(4-MeBzl)- |
| | OH•DCHA) |
| 368. | S-methyl-L-cysteine |
| 369. | α-methyl-DL-methionine |
| 370. | S-(2-(4-pyridyl)ethyl)-L-cysteine |
| 371. | S-(2-(4-pyridyl)ethyl)-DL-penicillamine |
| 372. | Di-seleno-L-cystine |
| 373. | L-selenomethionine |
| 374. | DL-selenomethionine |
| 375. | S-trityl-L-penicillamine |
| 376. | S-trityl-D-penicillamine |
| 377. | Di-L-cystathion |
| 378. | Di-DL-cystathionine |

Analogs of Serine, Threonine, and Statine

| | |
|---|---|
| 380. | 2-amino-3-methoxypropionic acid |
| 381. | L-α-methylserine |
| 382. | D-α-methylserine |
| 383. | (S)-2-amino-4-trityloxybutanoic acid (Hse(Trt)-OH) |
| 384. | (RS)-2-amino-4-trityloxybutanoic acid (DL-Hse(Trt)-OH) |
| 385. | (S)-2-amino-3-benzyloxypropionic acid |
| 386. | (R)-2-amino-3-benzyloxypropionic acid |
| 387. | (2S,3S)-2-amino-3-ethoxybutanoic acid |
| 388. | 2-amino-3-ethoxybutanoic acid |
| 389. | 2-amino-3-ethoxypropionic acid |
| 390. | 4-amino-3-hydroxybutanoic acid |
| 391. | (R)-2-amino-3-hydroxy-3-methylbutanoic acid |
| 392. | (S)-2-amino-3-hydroxy-3-methylbutanoic acid |
| 393. | (RS)-2-amino-3-hydroxy-3-methylbutanoic acid |
| 394. | (3S,4S)-4-amino-3-hydroxy-6-methylheptanoic acid (Sta-OH) |
| 395. | (2R,3R)-3-amino-2-hydroxy-5-methylhexanoic acid |
| 396. | (2R,3S)-3-amino-2-hydroxy-5-methylhexanoic acid |
| 397. | (2S,3R)-3-amino-2-hydroxy-5-methylhexanoic acid |
| 398. | (2S,3S)-3-amino-2-hydroxy-5-methylhexanoic acid |
| 399. | (2S,3R)-2-amino-3-hydroxy-4-methylpentanoic acid |
| 400. | (2R,3S)-2-amino-3-hydroxy-4-methylpentanoic acid |
| 401. | (2S,3RS)-2-amino-3-hydroxy-4-methylpentanoic acid |
| 402. | 2-amino-3-hydroxypentanoic acid |
| 403. | (2S,3R)-3-amino-2-hydroxy-4-phenylbutanoic acid |
| 404. | (2R,3R)-3-amino-2-hydroxy-4-phenylbutanoic acid |
| 405. | (2S,3S)-2-amino-3-methoxybutanoic acid |
| 406. | 2-amino-3-methoxybutanoic acid |
| 407. | (S)-2-amino-3-methoxypropionic acid |

Miscellaneous Aliphatic Amino Acids

| | |
|---|---|
| 409. | α-amino-1-adamantanepropionic acid |
| 410. | 2-aminobicyclo(2.2.1)heptane-2-carboxylic acid (mixture of isomers) |
| 411. | 3-endo-aminobicyclo(2.2.1)heptane-2-endo-carboxylic acid |
| 412. | 3-endo-aminobicyclo(2.2.1)heptane-2-endo-carboxylic acid |
| 413. | 3-endo-aminobicyclo(2.2.1)hept-5-ene-2-endo-carboxylic acid |
| 414. | 1-aminocyclobutane-1-carboxylic acid |
| 415. | 5-amino-1,3-cyclohexadiene-1-carboxylic acid |
| 416. | 1-aminocyclohexane-1-carboxylic acid |
| 417. | (±)-cis-2-aminocyclohexane-1-carboxylic acid |
| 418. | (±)-trans-2-aminocyclohexane-1-carboxylic acid |
| 419. | trans-4-aminocyclohexane-1-carboxylic acid |
| 420. | (±)-cis-3-aminocyclohexane-1-carboxylic acid |
| 421. | cis-4-aminocyclohexane-1-carboxylic acid |
| 422. | (±)-cis-2-aminocyclohex-4-ene-1-carboxylic acid |
| 423. | (±)-trans-2-aminocyclohex-4-ene-1-carboxylic acid |
| 424. | cis-4-aminocyclohexane-1-acetic acid |
| 425. | 1-aminocyclopentane-1-carboxylic acid |
| 426. | (±)-cis-2-aminocyclopentane-1-carboxylic acid |
| 427. | 1-aminocyclopropane-1-carboxylic acid |
| 428. | 2-aminoheptanoic acid |
| 429. | 7-aminoheptanoic acid |
| 430. | 6-aminohexanoic acid (6-aminocaproic acid) |
| 431. | 5-aminolevulinic acid |
| 432. | trans-4-(aminomethyl)cyclohexane-1-carboxylic acid |
| 433. | 2-aminooctanoic acid |
| 434. | 8-aminooctanoic acid (8-Aminocaprylic acid) |
| 435. | 3-(aminooxy)acetic acid |
| 436. | 5-aminopentanoic acid |
| 437. | 11-aminoundecanoic acid |

β-Amino Acids

| | |
|---|---|
| 439. | β-alanine (β-Ala-OH) |
| 440. | L-β-homoalanine (β-homoAla-OH) |
| 441. | (S)-N-ω-2,2,4,6,7-pentamethyldihydrobenzofuran-5-sulfonyl-L-β-homoarginine (β- |
| | homoArg(Pbf)-OH) |
| 442. | N-ω-tosyl-L-β-homoarginine (β-homoArg(Tos)-OH) |
| 443. | γ-trityl-L-β-homoasparagine (β-homoAsn(Trt)-OH) |
| 444. | L-β-homoaspartic acid γ-t-butyl ester (β-homoAsp(OtBu)-OH) |
| 445. | L-β-homoaspartic acid γ-benzyl ester (β-homoAsp(OBzl)-OH) |
| 446. | L-β-homoglutamic acid δ-t-butyl ester (β-homoGlu(OtBu)-OH) |
| 447. | L-β-homoglutamic acid δ-benzyl ester (β-homoGlu(OBzl)-OH) |
| 448. | N-δ-trityl-L-β-homoglutamine (β-homoGln(Trt)-OH) |
| 449. | O-t-butyl-L-β-homohydroxyproline (β-homoHyp(tBu)-OH) |
| 450. | L-β-homoisoleucine (β-homoIle-OH) |
| 451. | DL-β-leucine (DL-β-Leu-OH) |
| 452. | L-β-homoleucine (β-homoLeu-OH) |
| 453. | L-N-ω-β-homolysine (β-homoLys( )-OH) |
| 454. | L-N-ω-2-benzyloxycarbonyl-β-homolysine (β-homoLys(Z)-OH) |
| 455. | L-β-homomethionine (β-homoMet-OH) |
| 456. | L-β-phenylalanine (β-Phe-OH) |
| 457. | D-β-phenylalanine (D-β-Phe-OH) |
| 458. | L-β-homophenylalanine (β-homoPhe-OH) |
| 459. | L-β-homoproline (β-homoPro-OH) |
| 460. | O-t-butyl-L-β-homoserine (β-homoSer(tBu)-OH) |
| 461. | O-benzyl-L-β-homoserine (β-homoSer(Bzl)-OH) |
| 462. | O-benzyl-L-β-homothreonine (β-homoThr(Bzl)-OH) |
| 463. | L-β-homotryptophan (β-homoTrp-OH) |
| 464. | O-t-butyl-L-β-homotyrosine (β-homoTyr(tBu)-OH) |
| 465. | L-β-homovaline (β-homoVal-OH) |
| 466. | (R)-3-amino-4-(3-benzothienyl)butyric acid |
| 467. | (S)-3-amino-4-(3-benzothienyl)butyric acid |
| 468. | 3-aminobicyclo(2.2.2)octane-2-carboxylic acid (mixture of isomers) |
| 469. | (R)-3-amino-4-(4-bromophenyl)butyric acid |
| 470. | (S)-3-amino-4-(4-bromophenyl)butyric acid |
| 471. | (R)-3-amino-4-(2-chlorophenyl)butyric acid |
| 472. | (S)-3-amino-4-(2-chlorophenyl)butyric acid |
| 473. | (R)-3-amino-4-(3-chlorophenyl)butyric acid |
| 474. | (S)-3-amino-4-(3-chlorophenyl)butyric acid |
| 475. | (R)-3-amino-4-(4-chlorophenyl)butyric acid |
| 476. | (S)-3-amino-4-(4-chlorophenyl)butyric acid |
| 477. | 3-amino-3-(4-chlorophenyl)propionic acid |
| 478. | (R)-3-amino-4-(2-cyanophenyl)butyric acid |
| 479. | (S)-3-amino-4-(2-cyanophenyl)butyric acid |
| 480. | (R)-3-amino-4-(3-cyanophenyl)butyric acid |
| 481. | (S)-3-amino-4-(3-cyanophenyl)butyric acid |
| 482. | (R)-3-amino-4-(4-cyanophenyl)butyric acid |
| 483. | (S)-3-amino-4-(4-cyanophenyl)butyric acid |
| 484. | (R)-3-amino-4-(2,4-dichlorophenyl)butyric acid |
| 485. | (S)-3-amino-4-(2,4-dichlorophenyl)butyric acid |
| 486. | (R)-3-amino-4-(3,4-dichlorophenyl)butyric acid |
| 487. | (S)-3-amino-4-(3,4-dichlorophenyl)butyric acid |
| 488. | (R)-3-amino-4-(3,4-difluorophenyl)butyric acid |
| 489. | (S)-3-amino-4-(3,4-difluorophenyl)butyric acid |
| 490. | (R)-3-amino-4-(2-fluorophenyl)butyric acid |
| 491. | (S)-3-amino-4-(2-fluorophenyl)butyric acid |
| 492. | (R)-3-amino-4-(3-fluorophenyl)butyric acid |
| 493. | (S)-3-amino-4-(3-fluorophenyl)butyric acid |
| 494. | (R)-3-amino-4-(4-fluorophenyl)butyric acid |
| 495. | (S)-3-amino-4-(4-fluorophenyl)butyric acid |
| 496. | (R)-3-amino-4-(2-furyl)butyric acid |
| 497. | (S)-3-amino-4-(2-furyl)butyric acid |
| 498. | (R)-3-amino-5-hexenoic acid |
| 499. | (S)-3-amino-5-hexenoic acid |
| 500. | (R)-3-amino-5-hexynoic acid |
| 501. | (S)-3-amino-5-hexynoic acid |
| 502. | (R)-3-amino-4-(4-iodophenyl)butyric acid |
| 503. | (S)-3-amino-4-(4-iodophenyl)butyric acid |
| 504. | (R)-3-amino-4-(2-methylphenyl)butyric acid |
| 505. | (S)-3-amino-4-(2-methylphenyl)butyric acid |
| 506. | (R)-3-amino-4-(3-methylphenyl)butyric acid |
| 507. | (S)-3-amino-4-(3-methylphenyl)butyric acid |
| 508. | (R)-3-amino-4-(4-methylphenyl)butyric acid |
| 509. | (S)-3-amino-4-(4-methylphenyl)butyric acid |
| 510. | (R)-3-amino-4-(1-naphthyl)butyric acid |
| 511. | (S)-3-amino-4-(1-naphthyl)butyric acid |
| 512. | (R)-3-amino-4-(2-naphthyl)butyric acid |
| 513. | (S)-3-amino-4-(2-naphthyl)butyric acid |
| 514. | (R)-3-amino-4-(4-nitrophenyl)butyric acid |
| 515. | (S)-3-amino-4-(4-nitrophenyl)butyric acid |
| 516. | (R)-3-amino-4-pentafluorophenylbutyric acid |
| 517. | (S)-3-amino-4-pentafluorophenylbutyric acid |
| 518. | (R)-3-amino-6-phenyl-5-hexenoic acid |
| 519. | (S)-3-amino-6-phenyl-5-hexenoic acid |
| 520. | (R)-3-amino-5-phenylpentanoic acid |
| 521. | (S)-3-amino-5-phenylpentanoic acid |
| 522. | (R)-3-amino-4-(3-pyridyl)butyric acid |
| 523. | (S)-3-amino-4-(3-pyridyl)butyric acid |
| 524. | (R)-3-amino-4-(4-pyridyl)butyric acid |
| 525. | (S)-3-amino-4-(4-pyridyl)butyric acid |
| 526. | (R)-3-amino-4-(2-thienyl)butyric acid |
| 527. | (S)-3-amino-4-(2-thienyl)butyric acid |
| 528. | (R)-3-amino-4-(3-thienyl)butyric acid |
| 529. | (S)-3-amino-4-(3-thienyl)butyric acid |
| 530. | 3-amino-3-(2-thienyl)propionic acid |
| 531. | 3-amino-4,4,4-trifluorobutyric acid |
| 532. | (R)-3-amino-4-(2-trifluoromethylphenyl)butyric acid |
| 533. | (S)-3-amino-4-(2-trifluoromethylphenyl)butyric acid |
| 534. | (R)-3-amino-4-(3-trifluoromethylphenyl)butyric acid |
| 535. | (S)-3-amino-4-(3-trifluoromethylphenyl)butyric acid |
| 536. | (R)-3-amino-4-(4-trifluoromethylphenyl)butyric acid |
| 537. | (S)-3-amino-4-(4-trifluoromethylphenyl)butyric acid |
| 538. | (R)-1,2,3,4-tetrahydroisoquinoline-3-acetic acid |
| 539. | (S)-1,2,3,4-tetrahydroisoquinoline-3-acetic acid |
| 540. | 1,2,5,6-tetrahydropyridine-3-carboxylic acid (guvacine) |
| 541. | H-L-β-Homopro-OH HCl (S)-2-(2-Pyrrolidinyl) acetic acid hydrochloride |
| 542. | H-DL-β-Leu-OH (1)-3-Amino-4-methylpentanoic acid |
| 543. | H-DL-β-Homoleu-OH (1)-3-Amino-5-methylcaproic acid |
| 544. | H-DL-β-Phe-OH (1)-3-Amino-3-phenylpropionic acid |
| 545. | L-Homophe-OEt HCl |
| 546. | D-Homophe-OEt HCl |
| 547. | N-Benzyl-L-Homophe-OEt HCl |
| 548. | N-Benzyl-D-Homophe-OEt HCl |
| 549. | (1)-3-(amino)-4-(4-biphenylyl)butyric acid |
| 550. | (1)-3-Amino-4-(4-biphenylyl)butyric acid hydrochloride |
| 551. | (+)-Ethyl (S)-2-amino-4-cyclohexylbutyrate hydrochloride |
| 552. | (−)-Ethyl (R)-2-amino-4-cyclohexylbutyrate hydrochloride |

N-α-Methyl Amino Acids

| | |
|---|---|
| 554. | N-α-methyl-L-alanine (MeAla-OH) |
| 555. | N-α-methyl-D-alanine (D-MeAla-OH) |
| 556. | N-α-methyl-L-alloisoleucine (MeAlloIle-OH) |
| 557. | N-α-methyl-D-alloisoleucine (D-MeAlloIle-OH) |
| 558. | N-α-methyl-N-ω-tosyl-L-arginine (MeArg(Tos)-OH) |
| 559. | N-α-methyl-N-ω-2,2,4,6,7-pentamethyldihydrobenzofuran-5-sulfonyl-D-arginine |
| | (D-MeArg(Pbf)-OH) |
| 560. | N-α-methyl-N-ω-tosyl-D-arginine (D-MeArg(Tos)-OH) |
| 561. | N-α-methyl-L-aspartic acid |
| 562. | N-α-methyl-L-aspartic acid β-t-butyl ester (MeAsp(OtBu)-OH) |
| 563. | N-α-methyl-D-aspartic acid |
| 564. | N-α-methyl-D-aspartic acid β-t-butyl ester (D-MeAsp(OtBu)-OH) |
| 565. | N-α-methyl-4-chloro-L-phenylalanine (Me(4-Cl-Phe)-OH) |
| 566. | N-α-methyl-4-chloro-D-phenylalanine (D-Me(4-Cl-Phe)-OH) |
| 567. | N-α-methyl-L-glutamic acid γ-t-butyl ester (MeGlu(OtBu)-OH) |
| 568. | N-α-methyl-D-glutamic acid γ-t-butyl ester (D-MeGlu(OtBu)-OH) |
| 569. | N-α-methylglycine (sarcosine; Sar-OH) |
| 570. | N-α-methyl-N-im-trityl-L-histidine (MeHis(Trt)-OH) |
| 571. | N-α-methyl-N-im-trityl-D-histidine (D-MeHis(Trt)-OH) |
| 572. | N-α-methyl-trans-L-4-hydroxyproline |
| 573. | N-α-methyl-L-isoleucine (MeIle-OH) |
| 574. | N-α-methyl-L-leucine (MeLeu-OH) |
| 575. | N-α-methyl-D-leucine (D-MeLeu-OH) |
| 576. | N-α-methyl-N-ε-t-L-lysine (MeLys( )-OH) |
| 577. | N-α-methyl-N-ε-2-chlorobenzyloxycarbonyl-L-lysine (MeLys(2-Cl-Z)-OH) |
| 578. | N-α-methyl-4-nitro-L-phenylalanine (MePhe(4-NO2)-OH) |
| 579. | N-α-methyl-L-norleucine (MeNle-OH) |
| 580. | N-α-methyl-L-norvaline (MeNva-OH) |
| 581. | N-α-methyl-L-phenylalanine (MePhe-OH) |
| 582. | N-α-methyl-D-phenylalanine (D-MePhe-OH) |
| 583. | N-α-methyl-L-phenylglycine (MePhg-OH) |
| 584. | N-α-methyl-L-proline |
| 585. | N-α-methyl-O-benzyl-L-serine (MeSer(Bzl)-OH) |
| 586. | N-α-methyl-O-benzyl-L-serine dicyclohexylammonium salt (MeSer(Bzl)- |
| | OH•DCHA) |
| 587. | N-α-methyl-O-t-butyl-L-serine (MeSer(tBu)-OH) |
| 588. | N-α-methyl-O-t-butyl-L-threonine (MeThr(tBu)-OH) |
| 589. | N-α-methyl-L-tryptophan (MeTrp-OH) |
| 590. | N-α-methyl-DL-tryptophan (DL-MeTrp-OH) |
| 591. | N-α-methyl-O-benzyl-L-tyrosine (MeTyr(Bze-OH) |
| 592. | N-α-methyl-O-t-butyl-L-tyrosine (MeTyr(tBu)-OH) |
| 593. | N-α-methyl-O-methyl-L-tyrosine (MeTyr(Me)-OH) |
| 594. | N-α-methyl-O-benzyl-D-tyrosine (D-MeTyr(Bzl)-OH) |
| 595. | N-α-methyl-L-valine (MeVal-OH) |
| 596. | N-α-methyl-D-valine (D-MeVal-OH) |

Amino Alcohols

| | |
|---|---|
| 598. | L-alaninol |
| 599. | D-alaninol |
| 600. | 2-aminobenzylalcohol |
| 601. | 3-aminobenzylalcohol |
| 602. | 4-aminobenzylalcohol |
| 603. | (R)-(−)-2-aminobutanol |
| 604. | (S)-(+)-2-aminobutanol |
| 605. | 4-aminobutanol |
| 606. | 4-amino-2-butanol |
| 607. | 2-amino-5-chlorobenzylalcohol |
| 608. | (±)-cis-2-aminocyclohexanol |
| 609. | (±)-trans-2-aminocyclohexanol |
| 610. | trans-4-aminocyclohexanol |
| 611. | (1R,2S)-(−)-2-amino-1,2-diphenylethanol |
| 612. | (1S,2R)-(+)-2-amino-1,2-diphenylethanol |
| 613. | 2-(2-aminoethoxy)ethanol |
| 614. | α-(1-aminoethyl)-4-hydroxybenzyl alcohol |
| 615. | 2-amino-2-ethyl-1,3-propanediol |
| 616. | 6-aminohexanol |
| 617. | 1-amino-4-(2-hydroxyethyl)piperazine |
| 618. | (1R,2S)-(+)-cis-1-amino-2-indanol |
| 619. | (1S,2R)-(−)-cis-1-amino-2-indanol |
| 620. | (1S,2R)-(+)-2-amino-3-methoxyphenylpropanol |
| 621. | (±)-cis-2-aminomethylcycloheptanol |
| 622. | (±)-1-aminomethylcyclohexanol |
| 623. | (±)-cis-2-aminomethylcyclohexanol |
| 624. | (±)-trans-2-aminomethylcyclohexanol |
| 625. | (±)-cis-2-aminomethylcyclooctanol |
| 626. | 6-amino-2-methyl-2-heptanol (heptaminol) |
| 627. | α-aminomethyl-3-hydroxybenzyl alcohol (norphenylephrine) |
| 628. | α-aminomethyl-4-hydroxybenzyl alcohol (octopamine) |
| 629. | α-aminomethyl-4-hydroxy-3-methoxybenzyl alcohol (normetaephrine) |
| 630. | 2-amino-2-methyl-1,3-propanediol |
| 631. | 2-amino-2-methylpropanol (β-aminoisobutanol) |
| 632. | (1R,2R)-(−)-2-amino-1-(4-nitrophenyl)-1,3-propanediol |
| 633. | (1S,2S)-(+)-2-amino-1-(4-nitrophenyl)-1,3-propanediol |
| 634. | 5-aminopentanol |
| 635. | 1-amino-3-phenoxy-2-propanol |
| 636. | (R)-(−)-2-amino-1-phenylethanol |
| 637. | (S)-(+)-2-amino-1-phenylethanol |
| 638. | 2-(4-aminophenyl)ethanol |
| 639. | (1R,2R)-(−)-2-amino-1-phenyl-1,3-propanediol |
| 640. | (1S,2S)-(+)-2-amino-1-phenyl-1,3-propanediol |
| 641. | 3-amino-3-phenylpropanol |
| 642. | (RS)-3-amino-1,2-propanediol |
| 643. | (S)-(+)-3-amino-1,2-propanediol |
| 644. | (R)-(−)-1-amino-2-propanol |
| 645. | (S)-(+)-1-amino-2-propanol |
| 646. | 3-amino-1-propanol |
| 647. | N-ω-2,2,4,6,7-pentamethyldihydrobenzofuran-5-sulfonyl-L-argininol (Arg(Pbf)-ol) |
| 648. | N-ω-tosyl-L-argininol |
| 649. | N-β-trityl-L-asparaginol (Asn(Trt)-ol) |
| 650. | L-asparaginol (Asn-ol) |
| 651. | N-β-trityl-D-asparaginol (D-Asn(Trt)-ol) |
| 652. | D-asparaginol (D-Asn-ol) |
| 653. | L-aspartimol β-t-butyl ester (Asp(OtBu)-ol) |
| 654. | D-aspartimol β-t-butyl ester (D-Asp(OtBu)-ol) |
| 655. | DL-4-chlorophenylalaninol |
| 656. | β-cyclohexyl-L-alaninol |
| 657. | S-t-butyl-L-cysteinol (Cys(tBu)-ol) |
| 658. | S-t-butyl-D-cysteinol (D-Cys(tBu)-ol) |
| 659. | 1,1-diphenyl-L-alaninol |
| 660. | L-glutaminol (Gln-ol) |
| 661. | N-γ-trityl-L-glutaminol (Gln(Trt)-ol) |
| 662. | L-glutamol γ-t-butyl ester (Glu(OtBu)-ol) |
| 663. | L-glutamol γ-benzyl ester (Glu(OBzl)-ol) |
| 664. | D-glutamol γ-t-butyl ester (D-Glu(OtBu)-ol) |
| 665. | D-glutamol γ-benzyl ester (D-Glu(OtBu)-ol) |
| 666. | ethanolamine (Gly-ol) |
| 667. | N-im-t-L-histidinol |
| 668. | N-im-trityl-L-histidinol |
| 669. | N-im-benzyl-L-histidinol |
| 670. | 1-hydroxyethylethoxypiperazine |
| 671. | N-(2-hydroxyethyl)piperazine |
| 672. | N-(2-hydroxyethyl)-1,3-propanediamine |
| 673. | 3-endo-hydroxymethylbicyclo(2.2.1)hept-5-enyl-2-endo-amine |
| 674. | (±)-cis-2-hydroxymethyl-4-cyclohexenyl-1-amine |
| 675. | (±)-cis-2-hydroxymethyl-1-cyclohexylamine |
| 676. | (±)-trans-2-hydroxymethyl-1-cyclohexylamine |
| 677. | (±)-cis-2-hydroxymethyl-trans-4-phenyl-1-cyclohexylamine |
| 678. | 3-hydroxypiperidine |
| 679. | 4-hydroxypiperidine |
| 680. | L-isoleucinol (lle-ol) |
| 681. | L-leucinol (leu-ol) |
| 682. | D-leucinol (D-leu-ol) |
| 683. | L-tert-leucinol ((S)-(−)-2-amino-3,3-dimethyl-1-butanol) |
| 684. | N-ε-t-L-lysinol (Lys( )-ol) |
| 685. | N-ε-benzyloxycarbonyl-L-lysinol (Lys(Z)-ol) |
| 686. | N-ε-2-cholorobenzyloxycarbonyl-L-lysinol (Lys(2-CI-Z)-ol) |
| 687. | N-ε-t-D-lysinol (D-Lys( )-ol) |
| 688. | N-ε-benzyloxycarbonyl-D-lysinol (D-Lys(Z)-ol) |
| 689. | N-ε-2-cholorobenzyloxycarbonyl-D-lysinol (D-Lys(2-Cl-Z)-ol) |
| 690. | L-methioninol (Met-ol) |
| 691. | D-methioninol (D-Met-ol) |
| 692. | (1R,2S)-(−)-norephedrine |
| 693. | (1S,2R)-(+)-norephedrine |
| 694. | L-norleucinol |
| 695. | L-norvalinol |
| 696. | L-phenylalaninol |
| 697. | D-phenylalaninol (D-Phe-ol) |
| 698. | L-phenylglycinol (Phg-ol) |
| 699. | D-phenylglycinol (D-Phg-ol) |
| 700. | 2-(2-piperidyl)ethanol |
| 701. | 2-(4-piperidyl)ethanol |
| 702. | 2-piperidylmethanol |
| 703. | L-prolinol (Pro-ol) |
| 704. | D-prolinol (D-Pro-ol) |
| 705. | O-benzyl-L-serinol (Ser(Bzl)-ol) |
| 706. | O-t-butyl-L-serinol (Ser(tBu)-ol) |
| 707. | O-benzyl-D-serinol (D-Ser(Bzl)-ol) |
| 708. | O-t-butyl-D-serinol (D-Ser(tBu)-ol) |
| 709. | O-butyl-L-threoninol (Thr(tBu)-ol) |
| 710. | O-t-butyl-D-threoninol (Thr(tBu)-ol) |
| 711. | O-butyl-D-threoninol (Thr(tBu)-ol) |
| 712. | L-tryptophanol (Trp-ol) |
| 713. | D-tryptophanol (D-Trp-ol) |
| 714. | O-benzyl-L-tyrosinol (Tyr(Bzl)-ol) |
| 715. | O-t-butyl-L-tyrosinol (Tyr(tBu)-ol) |
| 716. | O-benzyl-D-tyrosinol (D-Tyr(Bzl)-ol) |
| 717. | L-valinol (Val-ol) |
| 718. | D-valinol (D-Val-ol) |

Others

| | |
|---|---|
| 720. | Norleucine |
| 721. | Ethionine |
| 722. | Ornithine |
| 723. | Thi-OH (−)-(R)-4-thiazolidine-carboxylic acid |
| 724. | 2-phosphonoglycine trimethyl ester |
| 725. | iminodiacetic acid |
| 726. | (1)-2-Aminoheptanedioic acid |
| 727. | (1)-2-Aminopimelic acid |
| 728. | 2-(2-(amino)ethoxy)ethoxy}acetic acid |
| 729. | 8-(amino)-3,6-dioxaoctanoic acid |
| 730. | 1-azetidine-3-carboxylic acid |
| 731. | (1R,4S)-(+)-4-(amino)-2-cyclopentene-1-carboxylic acid |
| 732. | cycloleucine |
| 733. | homocycloleucine |
| 734. | Freidinger's lactam |
| 735. | 1,2,3,4-tetrahydronorharman-3-carboxylic acid |
| 736. | 4-(aminomethyl)benzoic acid |
| 737. | 3-(aminomethyl)benzoic acid |
| 738. | 4-Abz-OH 4-(amino)benzoic acid |
| 739. | 3-Abz-OH 3-(amino)benzoic acid |
| 740. | 2-Abz-OH 2-(amino)benzoic acid |
| 741. | 2-(amino)isobutyric acid |
| 742. | 12-(amino)dodecanoic acid |
| 743. | 8-(amino)caprylic acid |
| 744. | 7-(amino)enanthic acid |
| 745. | 6-(amino)caproic acid |
| 746. | 5-(amino)pentanoic acid |
| 747. | 4-(amino)butyric acid |
| 748. | N′-diaminoacetic acid |
| 749. | L-2,3-diaminopropionic acid |
| 750. | N-β-L-2,3-diaminopropionic acid |
| 751. | (R)-4-(amino)-3-(Z-amino)butyric acid |
| 752. | (S)-4-(amino)-3-(Z-amino)butyric acid |
| 753. | 1,6-hexanediamine HCl |
| 754. | -1,5-pentanediamine |
| 755. | N-p-phenylenediamine |
| 756. | N-1,4-butanediamine |
| 757. | N-1,3-propanediamine |
| 758. | N-ethylenediamine |
| 759. | N-N-methylethylenediamine |
| 760. | 1-piperazine |
| 761. | 1-homopiperazine |

1. An isolated immunomodulatory bacteriocin or lantibiotic peptide with net cationic charge.

2. The peptide of claim 1 wherein the peptide has an amino acid sequence of SEQ ID NO: 1-6, or analogs, derivatives, amidated variations and conservative variations thereof.

3. An isolated polynucleotide that encodes a peptide of claim 1.

4. A method of selectively enhancing innate immunity comprising contacting a cell containing a gene that encodes a polypeptide involved in innate immunity and protection against an infection with an isolated immunomodulatory bacteriocin or lantibiotic peptide with net cationic charge, wherein expression of the gene in the presence of the bacteriocin or lantibiotic peptide is modulated as compared with expression of the gene in the absence of the bacteriocin or lantibiotic peptide, and wherein the modulated expression results in enhancement of innate immunity.

5. The method of claim 4 where the bacteriocin or lantibiotic peptide protects against an infectious agent.

6. The method of claim 5 where the infectious agent is a bacterium.

7. The method of claim 6, wherein the bacterium is selected from a group containing Staphylococcus aureus and Citrobacter rodentium.

8. The method of claim 4, wherein the innate immune response contributes to adjuvanticity leading to the promotion of a subsequent antibody response.

9. The method of claim 4, wherein the bacteriocin or lantibiotic peptide does not stimulate a septic reaction.

10. The method of claim 4, wherein the bacteriocin or lantibiotic peptide stimulates expression of the one or more genes or proteins and selectively enhances innate immunity.

11. The method of claim 4, wherein the one or more genes or proteins encode chemokines or interleukins that attract immune cells.

12. The method of claim 4, wherein one or more genes are selected from the group consisting of MCP-1, MCP-3, IL-8, Gro-α or IL-6.

13. The method of claim 4, wherein the peptide is a member of the cationic bacteriocin family.

14. The method of claim 13 wherein the bacteriocin is from the subfamily of cationic lantibiotics.

15. The method of claim 14, wherein the peptide is selected from the group consisting of SEQ ID NO: 1-6.

16. A method of selectively suppressing a proinflammatory response comprising contacting a cell containing a gene that encodes a polypeptide involved in inflammation and sepsis with an isolated immunomodulatory bacteriocin or lantibiotic peptide with net cationic charge, wherein the expression of the gene is modulated in the presence of the bacteriocin or lantibiotic peptide compared with expression in the absence of the bacteriocin or lantibiotic peptide, and wherein the modulated expression results in enhancement of innate immunity.

17. The method of claim 16, wherein the bacteriocin or lantibiotic peptide inhibits the inflammatory or septic response.

18. (canceled)

19. The method of claim 16, wherein the bacteriocin or lantibiotic peptide inhibits the expression of a pro-inflammatory gene or molecule.

20. The method of claim 16, wherein the bacteriocin or lantibiotic peptide inhibits the expression of TNF-α.

21. The method of claim 16, wherein the peptide is a member of the cationic bacteriocin family.

22. The method of claim 21, wherein the bacteriocin is from the subfamily of cationic lantibiotics.

23. The method of claim 22, wherein the peptide is selected from the group consisting of SEQ ID NO: 1-6.

24. The method of claim 16, wherein the inflammation is induced by a microbe or a microbial ligand acting on a Toll-like receptor.

25. The method of claim 24, wherein the microbial ligand is a bacterial endotoxin or lipopolysaccharide.

26. The method of claim 4, further comprising stimulating adaptive immune responses to immunization with an antigen.

27.-29. (canceled)

30. The method of claim 26, further comprising co-administering a conventional adjuvant.

31. The method of claim 30 wherein the conventional adjuvant is an oligonucleotide containing the sequence motif CpG.

32.-34. (canceled)

35. A method for identifying a compound which modulates an innate immune response, the method comprising:
(a) providing a cell-based assay system comprising a cell containing a gene that encodes a polypeptide involved in innate immunity and protection against infection, expression of the gene being modulated during an innate immune response;
(b) contacting the cell with a test compound; and
(c) measuring expression of the gene in the assay system, wherein a difference in expression in the presence of the compound relative to expression in the absence of the compound is indicative of modulation.

36. The method of claim 35 wherein the compound is an agonist of an innate immune response.

37. The method of claim 35 wherein the compound is an antagonist of an innate immune response.

38. The method of claim 35 wherein the compound is an inhibitor of an innate immune response.

39. The method of claim 35 wherein the compound is an activator of an innate immune response.

40. The method of claim 35, wherein the test compound is an organic molecule, a natural product, a peptide, an oligosaccharide, a nucleic acid, a lipid, an antibody, or binding fragment thereof.

41. (canceled)

42. The method of claim 35, wherein the test compound is from a random peptide library, a combinatorial library, an oligosaccharide library or a phage display library.

43. (canceled)

44. A pharmaceutical composition comprising a peptide of claim 1 a pharmaceutically acceptable carrier.

45. A pharmaceutical composition comprising a polynucleotide of claim 3 and a pharmaceutically acceptable carrier.
